SEQUENCE LISTING (8 sequences)

SEQ ID NO: 1 - DNA, 22 bp, Artificial Sequence
  Description of Artificial Sequence: Synthetic primer
tcacattgcagtattcgtgtgc

SEQ ID NO: 2 - DNA, 21 bp, Artificial Sequence
  Description of Artificial Sequence: Synthetic primer
catcccgtcgccttttattga

SEQ ID NO: 3 - DNA, 21 bp, Artificial Sequence
  Description of Artificial Sequence: Synthetic primer
agagctggacaatggatcggt

SEQ ID NO: 4 - DNA, 22 bp, Artificial Sequence
  Description of Artificial Sequence: Synthetic primer
gccttgctgatagccatgactc

SEQ ID NO: 5 - DNA, 19 bp, Artificial Sequence
  Description of Artificial Sequence: Synthetic primer
tgagtgccgtgtgtttcgg

SEQ ID NO: 6 - DNA, 21 bp, Artificial Sequence
  Description of Artificial Sequence: Synthetic primer
gttgctgaagttgcggttgag

SEQ ID NO: 7 - DNA, 19 bp, Artificial Sequence
  Description of Artificial Sequence: Synthetic primer
gtggccgctaccttcactg

SEQ ID NO: 8 - DNA, 19 bp, Artificial Sequence
  Description of Artificial Sequence: Synthetic primer
gccccactcctacttggtc

(19) United States
(12) Patent Application Publication
(10) Pub. No.: US 2023/0346831 A1
(43) Pub. Date: Nov. 2, 2023

(54) METHODS FOR TREATING METASTATIC CANCER USING LOW DOSE CARBON MONOXIDE

(71) Applicant: Cornell University, Ithaca, NY (US)

(72) Inventors: Yi-Chieh Du, Great Neck, NY (US); Augustine Choi, New York, NY (US)

(73) Assignee: Cornell University, Ithaca, NY (US)

(21) Appl. No.: 18/016,131

(22) PCT Filed: Jul. 15, 2021
(86) PCT No.: PCT/US2021/041833

§ 371 Date: Jan. 13, 2023

Related U.S. Application Data
(60) Provisional application No. 63/052,567, filed on Jul. 16, 2020.

(51) Int. Cl. A61K 33/00; A61K 31/295; A61K 31/30; A61K 45/06; A61P 35/04

(52) U.S. Cl. CPC A61K 33/00; A61K 31/295; A61K 31/30; A61K 45/06; A61P 35/04

(57) ABSTRACT

The present technology relates generally to methods for treating, preventing, and/or ameliorating metastasis in a subject in need thereof comprising administering to the subject a therapeutically effective amount of low dose carbon monoxide.

### CROSS-REFERENCE TO RELATED APPLICATIONS

This application claims the benefit of and priority to U.S. Provisional Appl. No. 63/052,567, filed Jul. 16, 2020, the disclosure of which is incorporated by reference herein in its entirety.

### TECHNICAL FIELD

The present technology relates generally to methods for treating, preventing, and/or ameliorating metastasis in a subject suffering from or diagnosed with cancer comprising administering to the subject a therapeutically effective amount of low dose carbon monoxide.

### BACKGROUND

The following description of the background of the present technology is provided simply as an aid in understanding the present technology and is not admitted to describe or constitute prior art to the present technology.

Metastasis is responsible for about 90% of cancer deaths and is very challenging to treat. Patients with localized cancer often have undetectable disseminated tumor cells, which could form metastatic tumors later. The mechanisms underlying metastasis, including the orchestrated programs coordinating the migration and dissemination of primary tumor cells to distal tissues, remain unclear (Lambert et al., Cell 168, 670-691 (2017)). Alteration of the TCA cycle and heme metabolism influences many biochemical pathways and may provide precursors implicated in the initiating steps of malignancy and the resistance of tumor cell sub-populations to anti-proliferative chemotherapies. Over-expression of certain genes, including the BCL-2 family proteins, may also enhance the metastatic potential of cancer cell lines.

Certain therapeutics block cancer cell proliferation, but do not decrease metastasis, and vice versa. For example, the role of the TGFβ pathway as a tumor-promoter or suppressor at the cancer cell level depends on its differential effects at the early and late stages of carcinogenesis. In early-stage tumors, the TGFβ pathway promotes cell cycle arrest and apoptosis. In contrast, at advanced stages, by promoting cancer cell motility, invasion, epithelial-to-mesenchymal transition, and cell sternness, the TGFβ pathway promotes tumor progression and metastasis (Neuzillet, et al., Pharmacol. Ther., 147, 22-31 (2015)). Suppressing STAT5 activity by ruxolitinib does not affect proliferation (Haricharan, et al., eLife, 2:e00996, 1-24 (2013)), but ruxolitinib can increase metastasis through effects on immune cells (Bottos, et al., Nat. Commun., 7:12258, 1-12 (2016)).

A deeper understanding of novel therapeutics on these underlying mechanisms has the potential to expand the repertoire of treatment strategies used to combat metastatic disease.

### SUMMARY

In one aspect, the present disclosure provides a method for treating or preventing metastasis in a subject in need thereof, comprising administering to the subject an effective amount of carbon monoxide at a low dose of about 100 ppm to about 500 ppm.

Additionally or alternatively, in some embodiments, the subject is diagnosed with or is suffering from breast cancer, lung and bronchus cancer, colon cancer, rectal cancer, prostate cancer, pancreatic cancer, liver cancer, kidney and renal cancer, brain and other nervous system tumors, head and neck cancer, neuroendocrine tumor, blood cancer, gynecologic malignancies, or urinary bladder cancer. Additionally or alternatively in some embodiments, breast cancer is an estrogen receptor positive (ER⁺) breast cancer, an estrogen receptor negative (ER⁻) breast cancer, a progesterone receptor positive breast cancer (PR⁺), a Her2⁺ breast cancer, or a triple negative (ER⁻/PR⁻/Her2⁻) breast cancer. Additionally or alternatively, in some embodiments, the subject exhibits at least one mutation in one or more genes selected from the group consisting of BARD1, BRCA1, BRCA2, PALB2, RAD51D, BRIP1, RAD51C, ESR1, BCL2, ABRAXAS1, AIP, ALK, APC, ATM, AXIN2, BAP1, BLM, BMPR1A, CASR, CDC73, CDH1, CDK4, CDKN1B, CDKN2A, CFTR, CHEK2, CPA1, CTNNA1, CTRC, DICER1, EGFR, EGLN1, EPCAM, FANCC, FH, FLCN, GALNT12, GREM1, HOXB13, KIF1B, KIT, LZTR1, MAX, MEN1, MET, MITF, MLH1, MLH3, MRE11, MSH2, MSH3, MSH6, MUTYH, NBN, NF1, NF2, NTHL1, PALLD, PDGFRA, PHOX2B, PMS2, POLD1, POLE, POT1, PRKAR1A, PRSS1, PTCH1, PTEN, RAD50, RB1, RECQL, RET, RINT1, RPS20, SDHA, SDHAF2, SDHB, SDHC, SDHD, SMAD4, SMARCA4, SMARCB1, SMARCE1, SPINK1, STK11, SUFU, TERT, TMEM127, TP53, TSC1, TSC2, VHL, and XRCC2.

Additionally or alternatively, in some embodiments, the metastasis has developed in one or more organs selected from the group consisting of lymph nodes, liver, brain, lungs, kidney, bones, lymphatics cavity, peritoneal cavity, and thoracic cavity. A subject may exhibit at least one symptom selected from among persistent cough, bloody phlegm, chest pain, shortness of breath, wheezing, weakness, sudden weight loss, bone pain, bone fractures, urinary incontinence, bowel incontinence, hypercalcemia, nausea, vomiting, constipation, confusion, headache, seizures, dizziness, numbness in the face, arms or legs, memory loss, changes in behaviour and personality, loss of balance and coordination, problems with speech and/or swallowing, abdominal pain, pain occurring near the right shoulder blade or in the upper abdomen, loss of appetite, abdominal swelling, jaundice, fatigue, and fever.

In any of the preceding embodiments of the methods disclosed herein, the effective amount of low dose carbon monoxide is about 100 ppm to about 500 ppm carbon monoxide. In certain embodiments, the subject may exhibit over-expression of HMMR or a Bcl-2 family gene, such as BCL2L1 (Bcl-xL).

Additionally or alternatively, in some embodiments, administration of the effective amount of carbon monoxide blocks metastasis and/or migration of breast cancer (e.g., an estrogen receptor negative (ER⁻) breast cancer, an estrogen receptor positive (ER⁺) breast cancer, a progesterone receptor positive breast cancer (PR⁺), a Her2⁺ breast cancer, or a triple-negative (ER⁻/PR⁻/Her2⁻) breast cancer), lung and bronchus cancer, colon cancer, rectal cancer, prostate cancer, pancreatic cancer, liver cancer, kidney and renal cancer, brain and other nervous system tumors, head and neck cancer, neuroendocrine tumor, blood cancer, gynecologic malignancies, or urinary bladder cancer, and/or does not reduce cancer cell proliferation. In certain embodiments, the subject is human.

In any of the above embodiments of the methods disclosed herein, the carbon monoxide is administered as or with at least one of a certified medical grade carbon monoxide gas, a recombumin-Ru^{II}(CO)₂ complex, a nanoparticle, or a carbon-monoxide releasing molecule (CORM).

In some embodiments, CORMs may include a transition-metal based CORM, an organic CORM, or a combination thereof. In some embodiments, the transition-metal based CORM may be a metal carbonyl complex of formula [M(CO)ₓL_{y}]_{±z}[Q]_{±p} wherein (i) M is a d transition metal, optionally Mo, Mn, Re, Fe, Ru, Co; (ii) x >1; (iii) L_{y} represents one or more ancillary mono-or polydentate ligands comprising C, N, O, P, S, Se, donor atoms or one or more of the halides, F, Cl, Br, I, which together with the CO ligands provide the complex with a 16, 17 or 18 electron valence shell configuration; (iv) z is the overall charge of the complex; (v) Q is a counter-ion; and (vi) p is an integer value such that the p± charge cancels the z± value. In some embodiments, the organic CORM may be an organoborane or an organic molecule configured to release CO to a biological medium or an entity-like buffer, a culture media, blood, a cell, a tissue, an organ, a tumor or a mammal. In some embodiments, the transition-metal based CORMs or the organic CORMs may release CO by at least one of: (i) spontaneous release upon dissolution; (ii) action of a specific chemical or enzymatic trigger in the cell, tissue, organ or tumor; (iii) exogenous action of another organic or inorganic chemical entity; or (iv) exogenous action of physical stimuli, optionally light, heat, electric or magnetic fields.

Examples of CORMs include, but are not limited to dichloromethane, sodium boranocarbonate, tricarbonyldichlororuthenium (II) dimer, tricarbonylchloro(glycinato)ruthenium (II), [Me₄N][Mn(CO)₄(thioacetate)₂], dimanganese decacarbonyl, iron pentacarbonyl, or any combination thereof. Examples of nanoparticles include, but are not limited to, liposomes, biodegradable polylactic acid (“PLA”), biodegradable polyglycolic acid (“PGA”), and biodegradable poly(lactic-co-glycolic acid) (“PGLA”).

In any of the preceding embodiments of the methods disclosed herein, administration of the effective amount of carbon monoxide results in decreased levels of one or more tricarboxylic acid (TCA) cycle metabolites in cancer cells compared to untreated cancer cells. Examples of the one or more TCA cycle metabolites include fumaric acid, L-Dihydroorotic acid, D-2-Hydroxyglutaric, malic acid, NAD, GDP-glucose, pyruvic acid, inosinic acid, cis-aconitate, succinic acid, succinyl-coA, and oxoglutaric acid. Additionally or alternatively, in some embodiments of the methods disclosed herein, administration of the effective amount of carbon monoxide results in reduced heme uptake or reduced heme biosynthesis in cancer cells compared to untreated cancer cells. In any of the above embodiments of the methods disclosed herein, administration of the effective amount of carbon monoxide results in decreased expression levels of HRG1, CYGB (Cytoglobin), CYP1B1 (Cytochrome P450 Family 1 Subfamily B Member 1), HCP1, SP1, WNT/beta-catenin, MYC, MYC target genes, and/or E2F target genes in cancer cells compared to untreated cancer cells.

In any and all embodiments of the methods disclosed herein, carbon monoxide is administered to the subject separately, sequentially or simultaneously with an additional therapeutic agent. Modes of administration for carbon monoxide and optionally any additional therapeutic agent include, but are not limited to, intravenous, intramuscular, intraarterial, intrathecal, intracapsular, intraorbital, intradermal, intraperitoneal, transtracheal, subcutaneous, intracerebroventricular, oral, topical, intratumoral, or intranasal administration.

Additional therapeutic agents can include, but are not necessarily limited to, alkylating agents, topoisomerase inhibitors, endoplasmic reticulum stress inducing agents, antimetabolites, immunotherapeutic agents, mitotic inhibitors, nitrogen mustards, nitrosoureas, alkylsulfonates, platinum agents, taxanes, vinca agents, anti-estrogen drugs, aromatase inhibitors, VEGF/VEGFR inhibitors, EGF/EGFR inhibitors, PARP inhibitors, cytostatic alkaloids, cytotoxic antibiotics, endocrine/hormonal agents, bisphosphonate therapy agents, phenphormin, anti-angiogenic agents, Histone deacetylase inhibitors, and non-steroidal anti-inflammatory drugs (NSAIDs).

Additionally or alternatively, in some embodiments, the additional therapeutic agents may comprise chemotherapeutic agents such as cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, ABRAXANE^{®} (albumin-bound paclitaxel), protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, alpelisib, and mixtures thereof.

Examples of antimetabolites include, but are not limited to, 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include, but are not limited to, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include, but are not limited to, cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitors include, but are not limited to, SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof.

Examples of topoisomerase II inhibitors include, but are not limited to, amsacrine, etoposide, etoposide phosphate, teniposide, daunorubicin, mitoxantrone, ellipticines, aurintricarboxylic acid, doxorubicin, and HU-331 and combinations thereof.

Examples of immunotherapeutic agents include, but are not limited to, immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

Examples of anti-angiogenic agents include, but are not limited to, bevacizumab, cediranib, axitinib, anginex, sunitinib, sorafenib, pazopanib, vatalanib, cabozantinib, ponatinib, lenvatinib, SU6668, Everolimus (Afinitor^{®}), Lenalidomide (Revlimid^{®}), Ramucirumab (Cyramza^{®}), Regorafenib (Stivarga^{®}), Thalidomide (Synovir, Thalomid^{®}), Vandetanib (Caprelsa^{®}), and Ziv-aflibercept (Zaltrap^{®}).

Examples of Histone deacetylase inhibitors include, but are not limited to, trichostatin A (TSA), tubacin, apicidin, depsipeptide, MS275, BNEL-210, RGFP966, MGCD0103, LBH589, splitomicin, FK228, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC202, Mocetinostat, Romidepsin, Nicotinamide, Sirtinol, Cambinol, and EX-527.

### BRIEF DESCRIPTION OF THE DRAWINGS

FIGS. 1A-1I show that low-dose CO treatment decreases cancer cell migration across different cancer types and supplement of hemin rescued this reduction. Migration of different cancer cells under control air or 250 ppm CO treatment with or without supplement of 30 µM hemin was determined using in vitro transwell migration chamber with a serum gradient. Indicated cell lines were seeded into the transwell inserts. 16 hours later, cells on the top of the upper chambers were removed, and cells on the bottom surface of the transwell inserts were fixed, stained with crystal violet, and counted from eight randomly picked fields in three independent experiments. Error bars represent SEM. *: P < 0.05, two-sided t-test. Scale bar, 100 µm. FIG. 1J shows a schematic representation of an in vitro transwell migration assay chamber to examine the effects of CO on tumor cell migration. Cells were seeded in the upper chamber of 8-µm porous polycarbonate membranes with DMEM containing 0% or 1% FBS, 0.2 mM L-glutamine, 1% penicillin/streptomycin. The lower chambers were filled with DMEM containing 10% or 20% FBS, 0.2 mM L-glutamine, and 1% penicillin/streptomycin (please see Table 1). FIG. 1K shows similar findings as FIG. 1A and FIG. 2A using MCF7-TGL-Bcl-xL cells, instead of MCF7/TGL/pQ cells. FIG. 1L shows 250 ppm CO only slightly reduced the migration of non-malignant BJ fibroblasts.

FIGS. 2A-2I show that low-dose CO treatment for 16 hours does not affect cancer cell proliferation across different cancer types. Proliferation of different cancer cells under control air or 250 ppm CO treatment with or without supplement of 30 µM hemin was determined. Indicated cell lines cell lines were seeded in the 24-well plate in normal growth media. 16 hours later, cells were fixed, stained with crystal violet, and lysed by methanol. OD595 were measured from triplicates of each cell type. Error bars represent SEM. *: P < 0.05, two-sided t-test. Scale bar, 100 µm. FIG. 2J shows a bar graph quantifying the effect of CO treatment on MDA-MB-231-TGL cell proliferation, illustrating that CO treatment had greater inhibitory effect on MDA-MB-231 cell proliferation when cells were seeded at higher density (6.25 × 10⁴ or 1.25 × 10⁵ cells per 24-well) than at lower density (3.125 × 10⁴ cells per 24-well).

FIGS. 3A-3H show that low-dose CO treatment decreases cancer metastasis in vivo. NSG mice were injected with 0.1 million (FIGS. 3A-3B) or 5 × 10⁴ (FIGS. 3C-3E) MDA-MB-231/TGL breast cancer cells through tail vein or 8988T/TGL pancreatic cancer cells through spleen (FIGS. 3F-3H). Mice were randomly divided into two groups after injection. One group of mice was kept in regular mouse holding room all the time while the other group was treated with 250 ppm CO for 3 hours daily starting one day after injection. FIGS. 3C and 3F: bioluminescent imaging was taken at day 0, day 1, day3 and then once a week and signals from the whole mice were plotted. FIGS. 3A, 3D and 3G: organs were harvested for histological analysis. Representative H&E stained images of metastatic tumors in the lung sections (FIGS. 3A and 3D) and in the liver sections (FIG. 3G) were shown. Metastatic tumor areas or numbers of mets on the H&E stained sections for each mouse were quantified (FIGS. 3B, 3E and 3H). FIG. 3I shows a representative image of a liver from an immunodeficient NOD/scid-IlL2Rgc knockout mouse (NSG) with tumors 24 days after 1.5 × 10⁶ N134/RHAMM^{B} cells in 120 µl PBS were injected into the mouse tail veins. The image shows N134/RHAMM^{B} cell metastasis in the liver in vivo. FIG. 3J shows a representative image of a liver from an immunodeficient NSG mouse with reduced tumor burden 24 days after 1.5 × 10⁶ N134/RHAMM^{B} cells in 120 µl PBS were injected into the mouse tail veins, which was subsequently treated daily for 3 hours with 250 ppm CO. FIG. 3K shows a dot plot quantifying the effects of carbon monoxide treatment on N134/RHAMM^{B} cell metastases dispersed on the liver of the injected mouse of FIGS. 3I-3J. FIG. 3L shows a dot plot quantifying the effects of carbon monoxide treatment on the tumor burden of each injected mouse 24 days following tail vein injections as shown in FIGS. 3I-3J, and demonstrates that carbon monoxide treatment showed a trend to reduce the total metastatic pancreatic tumor burden in the liver.

FIGS. 4A-4E show that low-dose CO downregulates intracellular heme levels. FIG. 4A: Western blot analysis for the level of HiF1α proteins in MCF7/TGL and MDA-MB-231/TGL cell lines with or without CO treatment, α-tubulin was used as a loading control. FIG. 4B: RNA-seq and GSEA analysis showed a positive correlation between CO treatment and heme metabolism in MCF7 and MDA231 cell lines. FIG. 4C: intracellular heme levels of MCF7/TGL and MDA-MB-231/TGL cells decreased after 250 ppm CO treatment, and supplement of hemin restored the heme reduction by CO. FIGS. 4D-4E: 250 ppm CO treatment decreased the expression level of two heme importers, HRG1 and HCP1 (FIG. 4D), but not heme exporter, FLVCR1 (FIG. 4D), HO-1 (FIG. 4E), and Bach1 (FIG. 4E).

FIGS. 5A-5C show that low-dose CO downregulated Myc target genes, CYP1B1, and Sp1. FIG. 5A: RNA-seq and GSEA analysis showed a negative correlation between CO treatment and Myc target gene sets (V1 and V2) in both MCF7/TGL and MDA-MB-231/TGL cell lines. FIG. 5B: 250 ppm CO reduced the mRNA levels of CYP1B1 and SP1. FIG. 5C: 250 ppm CO decreased CYP1B1 protein levels in MCF7/TGL, MDA-MB-231/TGL, and 8988T cell lines.

FIGS. 6A-6C show that transient expression of CYP1B1 restores breast cancer cell migration inhibited by CO. FIG. 6A: western blotting showed that CYP1B1 protein was increased in MCF7/TGL, MDA-MB-231/TGL, and 8988T cell lines 48 hours after transient transfection of a CYP1B1 expression vector. FIGS. 6B-6C: MCF7/TGL, MDA-MB-231/TGL, and 8988T cells with or without transient expression of CYP1B1 were subjected to 16-hour transwell migration assay and proliferation assay in the presence or absence of 250 ppm CO. Error bars represent SEM. *: P < 0.05, two-sided t-test. Scale bar, 100 µm.

FIG. 7 shows a metabolomics profiling cluster illustrating the effect of carbon monoxide treatment on metabolic changes in MDA-MB-231/TGL cells incubated with 250 ppm carbon monoxide for 16 hours. Carbon monoxide treatment reduced the abundance of the majority of metabolites tested. Cells were seeded in a 10 cm cell culture plate with DMEM containing 10% FBS, 0.2 mM L-glutamine and 1% penicillin/streptomycin. After 16 hours of incubation, metabolites were extracted from the cells using 80% methanol. The remaining steps were carried out with no change according to the methods of Goncalves et al, PNAS, 115:4, E743-E752 (2018). All data analyses were done using MetaboAnalyst.

FIG. 8 shows a metabolomics profiling cluster illustrating the effect of carbon monoxide treatment on metabolic changes in MCF7/TGL/pQ cells incubated with 250 ppm carbon monoxide for 16 hours. Carbon monoxide treatment reduced the abundance of the majority of metabolites tested. Extraction and analysis were performed with the same parameters as the method disclosed in FIG. 7.

FIG. 9 shows a pathway topology map of the downregulated metabolites of FIGS. 7 and 8, and highlights the impact of carbon monoxide treatment on the TriCarboxylic Acid (TCA) cycle pathway. The TCA cycle is the top metabolic pathway downregulated by CO in two types of breast cancer cell lines. See Table 3 and Table 4.

FIGS. 10A-10B show metabolomic profiling clusters illustrating the nine most downregulated metabolites following carbon monoxide treatment in MCF7 and MDA-MB-231 cells, respectively. Specifically, carbon monoxide treatment reduced the abundance of fumaric acid, L-Dihydroorotic acid, D-2-Hydroxyglutaric, malic acid, NAD, GDP-glucose, pyruvic acid, inosinic acid, and oxoglutaric acid in both MCF7 and MDA-MB-231 cells. FIG. 10C shows a pathway topology map of the nine most downregulated metabolites of FIGS. 10A-10B (see Table 5), and highlights the impact of carbon monoxide treatment on the TriCarboxylic Acid (TCA) cycle pathway. Pathway topology analysis using these nine compounds suggested that tricarboxylic acid (TCA) cycle was the most significantly downregulated pathway by 250 ppm CO.

FIG. 11A shows a pathway illustrating intermediate metabolites of the TCA cycle and their interrelationships. In FIGS. 11B-11I, all TCA intermediates detected in the polar metabolites profiling assay were compared. Besides pyruvic acid (FIG. 11B), oxoglutaric acid (FIG. 11E), malic acid (FIG. 11H), and fumaric acid (FIG. 11G), which were significantly downregulated by 250 ppm CO in both cell lines, succinic acid was significantly downregulated by CO in MCF7/TGL cells (FIG. 11F) and cis-aconitic acid (FIG. 11D) was significantly downregulated by 250 ppm CO in MDA-MB-231/TGL cells. Citric acid (FIG. 11C) and phosphoenolpyruvic acid (FIG. 11I) did not provide statistically significant results.

FIG. 12A shows a table describing six mouse cell lines with distinct metastatic ability. The table is adapted from Lu et al., J. Biol. Chem. 285(13):9317-9321(2010). FIG. 12B shows metabolomics profiling cluster demonstrating metabolites increased in most of the metastatic mouse cells shown in FIG. 12A. FIG. 12B is adapted from Lu et al., J. Biol. Chem. 285(13):9317-9321 (2010). FIG. 12C shows a schematic representation of a two-step metabolic change model involved in the progression from a normal cell to primary tumor cell (step 1) and metastatic tumor cell (step 2) (adapted from Lu et al., J. Biol. Chem. 285(13):9317-9321 (2010)).

FIG. 13A shows a bar graph quantifying mitochondrial ATP production in MDA-MB-231/TGL and MCF7/TGL/PQ cells, and illustrates that carbon monoxide treatment did not affect mitochondrial function in these cells. In particular, MDA-MB-231/TGL and MCF7/TGL/PQ cells treated with carbon monoxide met their energetic needs at the same rate as untreated control cells. FIG. 13B shows a bar graph quantifying mitochondrial maximum respiration in MDA-MB-231/TGL and MCF7/TGL/PQ cells, and illustrates that carbon monoxide treatment did not affect stress-induced mitochondrial maximum respiration rate. In particular, MDA-MB-231/TGL and MCF7/TGL/PQ cells treated with carbon monoxide stimulated their mitochondrial respiratory chains at the same rate as untreated control cells. FIG. 13C shows a bar graph quantifying mitochondrial spare respiratory capacity in MDA-MB-231/TGL and MCF7/TGL/PQ cells.

FIG. 14 shows a schematic model of carbon monoxide-mediated suppression of tumor migration, involving decreasing heme levels via the downregulation of heme transporter HRG1 (SLC48a1) and Heme Carrier Protein 1 (HCP1; SLC46A1) mRNA expression or the downregulation of the TCA cycle.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir’s Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Carbon monoxide (CO) is a non-corrosive gas of about the same density as that of air and is most commonly encountered as a poison. Depending on the extent and time of exposure, CO is capable of producing a myriad of debilitating and harmful residual effects to an organism. The most well-known of these effects, is binding to hemoglobin in the blood stream, which rapidly decreases the oxygen transport capability of the cardiovascular system. However, CO is also constantly formed in humans in small quantities, and under certain pathophysiological conditions this endogenous production of CO may be considerably increased. Hemoglobin, a heme-dependent protein, is required as substrate for the production of CO in vivo and the identification of the enzyme heme oxygenase as the crucial pathway for the generation of this gaseous molecule in mammals provides the basis for investigation of an unexpected and still unrecognized role of CO in vasculature and metastatic diseases.

The present disclosure demonstrates that in low, carefully controlled doses, carbon monoxide can mimick and enhance the therapeutic effects of certain chemotherapeutic agents by blocking migration of cancer cells, without affecting cancer cell proliferation per se. The present disclosure demonstrates that low dose carbon monoxide significantly reduces human cancer cell migration in vitro and human cancer metastasis in mouse models. As shown in FIG. 14, carbon monoxide administration decreases tumor migration by decreasing heme uptake via the downregulation of heme transporter HRG1 (SLC48a1) and Heme Carrier Protein 1 (HCP1; SLC46A1). Carbon monoxide also elicits downregulation of the TCA cycle, which in turn inhibits heme synthesis and lowers heme levels. One of the downstream reactions in the TCA cycle is the biosynthesis of heme, (iron protoporphyrin IX), an essential iron-containing molecule. Free heme (Fe²⁺ state) is readily oxidized, so oxidized heme levels in the form of hemin (Fe³⁺ state) are used to assess intracellular heme levels. The present disclosure demonstrates that low dose carbon monoxide treatment reduces the expression of heme transporters, HRG1 and HCP1, thus impairing heme uptake. Thus, carbon monoxide has an inhibitory effect on migration of cancer cells, and is useful in methods for reducing or eliminating metastatic cancers.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the terms “cancer,” “neoplasm,” and “tumor,” are used interchangeably and refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function as well as protein degradation/turnover.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al., 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, “low dose” refers to an effective amount of carbon monoxide, which in some embodiments may be any amount of carbon monoxide between about 150 ppm to about 750 ppm and any subvalues therebetween. In certain preferred embodiments, low dose refers to an effective amount comprising about 250 ppm of carbon monoxide (CO).

As used herein, the term “metastasis” or “metastatic” refers to the ability of a cancer cell to invade surrounding tissues, to enter the circulatory system and to establish malignant growths at new sites.

“Non-Metastatic” refers to tumors that do not spread beyond their original site of development and specifically do not enter the circulatory system and establish malignant growths at new sites.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington’s Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single-and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, “prevention,” “prevent,” or “preventing” of a disease or condition refers to one or more compounds that, in a statistical sample, reduces the occurrence of the disease or condition in the treated sample relative to an untreated control sample, or delays the onset of one or more symptoms of the disease or condition relative to the untreated control sample. As used herein, prevention includes preventing or delaying the initiation of symptoms of the disease or condition. As used herein, prevention also includes preventing a recurrence of one or more signs or symptoms of a disease or condition.

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### Therapeutic Compositions Including Carbon Monoxide

The present disclosure demonstrates that low dose carbon monoxide significantly reduces human cancer cell migration in vitro and metastatic animal models. Exposure of multiple cell lines to CO greatly reduced migration of the cells and metastatic profiles in both in vitro transwell migration assay and tail vein animal models. As shown in FIG. 14, carbon monoxide administration decreases tumor migration by decreasing heme uptake via the downregulation of heme transporter HRG1 (SLC48a1) and Heme Carrier Protein 1 (HCP1; SLC46A1). Carbon monoxide also elicits downregulation of the TCA cycle, which in turn inhibits heme synthesis and lowers heme levels. Accordingly, carbon monoxide has an inhibitory effect on migration of cancer cells, and is useful in methods for reducing or eliminating metastatic cancers.

For therapeutic and/or prophylactic applications, a composition comprising low dose carbon monoxide is administered to the subject. In some aspects of the disclosure, CO is administered in doses of between about 150 ppm to about 750 ppm and any value in between, including, for the avoidance of doubt, all non-whole number and non-integer values in parts per million as well as all whole number values and integer values in parts per million. In some embodiments, an effective amount of CO is about 150 ppm, about 155 ppm, about 160 ppm, about 165 ppm, about 170 ppm, about 175 ppm, about 180 ppm, about 185 ppm, about 190 ppm, about 195 ppm, about 200 ppm, about 205 ppm, about 210 ppm, about 215 ppm, about 220 ppm, about 225 ppm, about 230 ppm, about 235 ppm, about 240 ppm, about 245 ppm, about 250 ppm, about 255 ppm, about 260 ppm, about 265 ppm, about 270 ppm, about 275 ppm, about 280 ppm, about 285 ppm, about 290 ppm, about 295 ppm, about 300 ppm, about 305 ppm, about 310 ppm, about 315 ppm, about 320 ppm, about 325 ppm, about 330 ppm, about 335 ppm, about 340 ppm, about 345 ppm, about 350 ppm, about 355 ppm, about 360 ppm, about 365 ppm, about 370 ppm, about 375 ppm, about 380 ppm, about 385 ppm, about 390 ppm, about 395 ppm, about 300 ppm, about 305 ppm, about 310 ppm, about 315 ppm, about 320 ppm, about 325 ppm, about 330 ppm, about 335 ppm, about 340 ppm, about 345 ppm, about 350 ppm, about 355 ppm, about 360 ppm, about 365 ppm, about 370 ppm, about 375 ppm, about 380 ppm, about 385 ppm, about 390 ppm, about 395 ppm, about 400 ppm, about 405 ppm, about 410 ppm, about 415 ppm, about 420 ppm, about 425 ppm, about 430 ppm, about 435 ppm, about 440 ppm, about 445 ppm, about 450 ppm, about 455 ppm, about 460 ppm, about 465 ppm, about 470 ppm, about 475 ppm, about 480 ppm, about 485 ppm, about 490 ppm, about 495 ppm, about 500 ppm, about 505 ppm, about 510 ppm, about 515 ppm, about 520 ppm, about 525 ppm, about 530 ppm, about 535 ppm, about 540 ppm, about 545 ppm, about 550 ppm, about 555 ppm, about 560 ppm, about 565 ppm, about 570 ppm, about 575 ppm, about 580 ppm, about 585 ppm, about 590 ppm, about 595 ppm, about 600 ppm, about 605 ppm, about 610 ppm, about 615 ppm, about 620 ppm, about 625 ppm, about 630 ppm, about 635 ppm, about 640 ppm, about 645 ppm, about 650 ppm, about 655 ppm, about 660 ppm, about 665 ppm, about 670 ppm, about 675 ppm, about 680 ppm, about 685 ppm, about 690 ppm, about 695 ppm, about 700 ppm, about 705 ppm, about 710 ppm, about 715 ppm, about 720 ppm, about 725 ppm, about 730 ppm, about 735 ppm, about 740 ppm, about 745 ppm, or about 750 ppm. Values and ranges intermediate to the recited values are also contemplated as part of the present disclosure. In certain embodiments, an effective amount of CO is at a concentration of about 250 ppm.

In some embodiments, carbon monoxide is administered as at least one of certified medical grade carbon monoxide gas, a recombumin-Ru^{II}(CO)₂ complex, nanoparticles, or carbon-monoxide releasing molecules (CORMs). In some embodiments, the CORM may be a transition metal based CORM, an organic CORM, or a combination thereof. In some embodiments, the transition-metal based CORM may be a metal carbonyl complex of formula [M(CO)ₓL_{y}]_{±z}[Q]_{±p} wherein (i) M is a d transition metal, optionally Mo, Mn, Re, Fe, Ru, Co; (ii) x > 1; (iii) L_{y} represents one or more ancillary mono-or polydentate ligands comprising C, N, O, P, S, Se, donor atoms or one or more of the halides, F, Cl, Br, I, which together with the CO ligands provide the complex with a 16, 17 or 18 electron valence shell configuration; (iv) z is the overall charge of the complex; (v) Q is a counter-ion; and (vi) p is an integer value such that the p± charge cancels the z± value. Preferable ligands L may be those that carry or are conjugated to substituents acting as targeting vectors, namely bioactive natural substances, drugs and antibodies. Said metal carbonyl complex releases CO following administration to a biological medium or entity like buffer, culture medium, blood, cell, tissue, organ, tumor or mammal. Such release may become active in one or more of the following ways: spontaneous upon dissolution; by action of a specific chemical or enzymatic trigger in the cell, tissue, organ or tumor; by exogenous action of another organic or inorganic molecular entity; by exogenous action of physical stimuli namely light, heat, electric or magnetic fields.

In some embodiments, the organic CORM may be an organoborane or an organic molecule configured to release CO to a biological medium or an entity-like buffer, a culture media, blood, a cell, a tissue, an organ, a tumor or a mammal by at least one of: (i) spontaneous release upon dissolution; (ii) action of a specific chemical or enzymatic trigger in the cell, tissue, organ or tumor; (iii) exogenous action of another organic or inorganic chemical entity; or (iv) exogenous action of physical stimuli, optionallylight, heat, electric or magnetic fields.

A composite may be designed to deliver CO following administration to a biological medium or entity like buffer, culture media, blood, cell, tissue, organ, tumor or mammal. Said composite, or material, may be based on a molecular scaffold formed by large molecular entities or nanoparticles, capable to host in a supramolecular fashion or covalently bind CO, organic CORMs or metal-based CORMs. In some embodiments of the composite, delivery of CO may occur in three ways: (i) following structural collapse and liberation of CO or caged CORMs which are then activated by the same mechanisms that regulates CO release from said metal-based or organic CORMs; (ii) allowing the structurally exposed and covalently bound CORMs to be chemically activated in the same manner as their free counterparts or by the action of physical stimuli namely light, heat, electric or magnetic fields; or (iii) undergoing activation of the caged CORMs by invading chemical triggers by the action of physical stimuli, namely light, heat, electric or magnetic fields. Exemplary molecular scaffolds may include, among others, liposomes, functionalized micropolymers, micropolymer micelles, synthetic polymer fibres, polysaccharides and fibres thereof, metal organic frameworks, porous, mesoporous, microporous and hollow inorganic matrices, proteins and peptides, metal nanoparticles, or organic nanoparticles.

In some embodiments, the CORMs comprise dichloromethane, sodium boranocarbonate, tricarbonyldichlororuthenium (II) dimer, tricarbonylchloro(glycinato)ruthenium (II), [Me₄N][Mn(CO)₄(thioacetate)₂], dimanganese decacarbonyl, iron pentacarbonyl, or any combination thereof. In some embodiments, nanoparticles used for administration of the low dose carbon monoxide can include, but are not limited to, liposomes, biodegradable polylactic acid (“PLA”), biodegradable polyglycolic acid (“PGA”), biodegradable poly(lactic-co-glycolic acid) (“PGLA”), and ultrasound contrast microbubbles (Qin et al., Phys. Med. Biol., 54(6): R27 (2009)).

In some embodiments, the low dose carbon monoxide is administered one, two, three, four, or five times per day. In some embodiments, the low dose carbon monoxide is administered more than five times per day. Additionally or alternatively, in some embodiments, the carbon monoxide is administered every day, every other day, every third day, every fourth day, every fifth day, or every sixth day. In some embodiments, the low dose carbon monoxide is administered weekly, bi-weekly, tri-weekly, or monthly. In some embodiments, the low dose carbon monoxide is administered for a period of one, two, three, four, or five weeks. In some embodiments, the low dose carbon monoxide is administered for six weeks or more. In some embodiments, the low dose carbon monoxide is administered for twelve weeks or more. In some embodiments, the low dose carbon monoxide is administered for a period of less than one year. In some embodiments, the low dose carbon monoxide is administered for a period of more than one year. In some embodiments, the low dose carbon monoxide is administered throughout the subject’s life.

In some embodiments, the carbon monoxide is administered daily for 1 week or more. In some embodiments of the methods of the present technology, the low dose carbon monoxide is administered daily for 2 weeks or more. In some embodiments of the methods of the present technology, the low dose carbon monoxide is administered daily for 3 weeks or more. In some embodiments of the methods of the present technology, the low dose carbon monoxide is administered daily for 4 weeks or more. In some embodiments of the methods of the present technology, the low dose carbon monoxide is administered daily for 6 weeks or more. In some embodiments of the methods of the present technology, the low dose carbon monoxide is administered daily for 12 weeks or more. In some embodiments, the low dose carbon monoxide is administered daily throughout the subject’s life. In some embodiments of the methods of the present technology, the low dose carbon monoxide is administered over a period of about 1 hour to about 24 hours. In certain embodiments, the low dose carbon monoxide is administered over a period of about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. Values and ranges intermediate to the recited values are also contemplated as part of the present disclosure.

### Methods of the Present Technology

The following discussion is presented by way of example only, and is not intended to be limiting.

In one aspect, the present disclosure provides a method for treating or preventing metastasis in a subject in need thereof, comprising administering to the subject an effective amount of low dose carbon monoxide. In some embodiments, the subject is diagnosed with or is suffering from cancer. Examples of cancers include, but are not limited to, breast cancer, bladder cancer, cervical cancer, childhood cancers, colorectal cancer, endometrial cancer, esophageal cancer, ganglioneuroma, gastric cancer, glioma, hepatic cancer, kidney cancer, lung cancer, malignant peripheral nerve sheath tumor (MPNST), medullary thyroid carcinoma, melanoma, neuroblastoma, ovarian cancer, pancreatic cancer, pheochromocytoma, prostate cancer, testicular cancer, thyroid cancer, uterine cancer, brain tumor, sarcoma, lymphoma, and leukemia.

In certain embodiments, the subject is diagnosed with or is suffering from breast cancer, lung and bronchus cancer, colon cancer, rectal cancer, prostate cancer, pancreatic cancer, liver cancer, kidney and renal cancer, brain and other nervous system tumors, head and neck cancer, neuroendocrine tumor, blood cancer, gynecologic malignancies, or urinary bladder cancer. Additionally or alternatively, in some embodiments, the breast cancer is an estrogen receptor negative (ER⁻) breast cancer, an estrogen receptor positive (ER⁺) breast cancer, a progesterone receptor negative (PR⁻) breast cancer, a progesterone receptor positive (PR⁺) breast cancer, a Her2⁺ breast cancer, or a triple negative (ER⁻/PR⁻/Her2⁻) breast cancer. Additionally or alternatively, in some embodiments, the subject exhibits at least one mutation in one or more genes selected from the group consisting of BARD1, BRCA1, BRCA2, PALB2, RAD51D, BRIP1, RAD51C, BCL2, BCL2L1, ESR1, ABRAXAS1, AIP, ALK, APC, ATM, AXIN2, BAP1, BLM, BMPR1A, CASR, CDC73, CDH1, CDK4, CDKN1B, CDKN2A, CFTR, CHEK2, CPA1, CTNNA1, CTRC, DICER1, EGFR, EGLN1, EPCAM, FANCC, FH, FLCN, GALNT12, GREM1, HOXB13, KIF1B, KIT, LZTR1, MAX, MEN1, MET, MITF, MLH1, MLH3, MRE11, MSH2, MSH3, MSH6, MUTYH, NBN, NF1, NF2, NTHL1, PALLD, PDGFRA, PHOX2B, PMS2, POLD1, POLE, POT1, PRKAR1A, PRSS1, PTCH1, PTEN, RAD50, RB1, RECQL, RET, RINT1, RPS20, SDHA, SDHAF2, SDHB, SDHC, SDHD, SMAD4, SMARCA4, SMARCB1, SMARCE1, SPINK1, STK11, SUFU, TERT, TMEM127, TP53, TSC1, TSC2, VHL, XRCC2, genes involved in the mitogen-activated protein kinase (MAPK) pathway, p53 pathway, WNT pathway, TNF pathway, and estrogen receptor transcriptional regulators (MYC, CTCF, FOXA1, and TBX3).

Additionally or alternatively, in some embodiments, the metastasis has developed in one or more organs selected from the group consisting of lymph nodes, liver, brain, lungs, kidney, bones, lymphatics cavity, peritoneal cavity, and thoracic cavity. Additionally or alternatively, in some embodiments, the subject exhibits at least one symptom selected from the group consisting of persistent cough, bloody phlegm, chest pain, shortness of breath, wheezing, weakness, sudden weight loss, bone pain, bone fractures, urinary incontinence, bowel incontinence, hypercalcemia, nausea, vomiting, constipation, confusion, headache, seizures, dizziness, numbness in the face, arms or legs, memory loss, changes in behaviour and personality, loss of balance and coordination, problems with speech and/or swallowing, abdominal pain, pain occurring near the right shoulder blade or in the upper abdomen, loss of appetite, abdominal swelling, jaundice, fatigue, and fever. In any and all embodiments of the methods disclosed herein, treatment with low dose carbon monoxide will treat or ameliorate one or more symptoms selected from the group consisting of persistent cough, bloody phlegm, chest pain, shortness of breath, wheezing, weakness, sudden weight loss, bone pain, bone fractures, urinary incontinence, bowel incontinence, hypercalcemia, nausea, vomiting, constipation, confusion, headache, seizures, dizziness, numbness in the face, arms or legs, memory loss, changes in behaviour and personality, loss of balance and coordination, problems with speech and/or swallowing, abdominal pain, pain occurring near the right shoulder blade or in the upper abdomen, loss of appetite, abdominal swelling, jaundice, fatigue, and fever.

Additionally or alternatively, in some embodiments, administration of the effective amount of carbon monoxide blocks metastasis and/or migration of breast cancer, lung and bronchus cancer, colon cancer, rectal cancer, prostate cancer, pancreatic cancer, liver cancer, kidney and renal cancer, brain and other nervous system tumors, head and neck cancer, neuroendocrine tumor, blood cancer, gynecologic malignancies, or urinary bladder cancer, and/or does not reduce cancer cell proliferation. Additionally or alternatively, in some embodiments, administration of the effective amount of carbon monoxide blocks migration, metastases and/or proliferation in triple negative breast cancer cells or liver cancer cells. In certain embodiments, the subject is human.

In any of the preceding embodiments of the methods disclosed herein, administration of the effective amount of carbon monoxide results in decreased levels of one or more tricarboxylic acid (TCA) cycle metabolites in cancer cells compared to untreated cancer cells. Examples of the one or more TCA cycle metabolites include fumaric acid, L-Dihydroorotic acid, D-2-Hydroxyglutaric, malic acid, NAD, GDP-glucose, pyruvic acid, inosinic acid, cis-aconitate, succinic acid, succinyl-coA, and oxoglutaric acid. Additionally or alternatively, in some embodiments of the methods disclosed herein, administration of the effective amount of carbon monoxide results in reduced heme uptake or reduced heme biosynthesis in cancer cells compared to untreated cancer cells. In any of the above embodiments of the methods disclosed herein, administration of the effective amount of carbon monoxide results in decreased expression levels of HRG1, CYGB (Cytoglobin), CYP1B1 (Cytochrome P450 Family 1 Subfamily B Member 1), HCP1, SP1, WNT/beta-catenin, MYC, MYC target genes, and/or E2F target genes in cancer cells compared to untreated cancer cells.

In any and all embodiments of the methods disclosed herein, the low dose carbon monoxide can be administered as a certified medical grade carbon monoxide gas, a recombumin-Ru^{II}(CO)₂ complex, a nanoparticle, or a carbon-monoxide releasing molecule (CORM). In any of the preceding embodiments of the methods disclosed herein, carbon monoxide is administered to the subject separately, sequentially or simultaneously with an additional therapeutic agent. Modes of administration for carbon monoxide and optionally any additional therapeutic agent include, but are not limited to, intravenous, intramuscular, intraarterial, intrathecal, intracapsular, intraorbital, intradermal, intraperitoneal, transtracheal, subcutaneous, intracerebroventricular, oral, topical, intratumoral, or intranasal administration.

In one aspect, the present technology provides a method for preventing or delaying the onset of metastasis of an epithelial cancer. Examples of epithelial cancers include, but are not limited to, breast cancer, bladder cancer, cervical cancer, childhood cancers, colorectal cancer, endometrial cancer, esophageal cancer, ganglioneuroma, gastric cancer, glioma, hepatic cancer, kidney cancer, lung cancer, malignant peripheral nerve sheath tumor (MPNST), medullary thyroid carcinoma, melanoma, neuroblastoma, ovarian cancer, pancreatic cancer, pheochromocytoma, prostate cancer, testicular cancer, thyroid cancer, uterine cancer, brain tumor, sarcoma, lymphoma, and leukemia.

Administration of low dose carbon monoxide can occur prior to the manifestation of symptoms characteristic of the metastatic disease or disorder, such that the disease or disorder is prevented or, alternatively, delayed in its progression.

In some embodiments, treatment with the low dose carbon monoxide will prevent or delay the onset of one or more of the following symptoms: persistent cough, bloody phlegm, chest pain, shortness of breath, wheezing, weakness, sudden weight loss, bone pain, bone fractures, urinary incontinence, bowel incontinence, hypercalcemia, nausea, vomiting, constipation, confusion, headache, seizures, dizziness, numbness in the face, arms or legs, memory loss, changes in behaviour and personality, loss of balance and coordination, problems with speech and/or swallowing, abdominal pain, pain occurring near the right shoulder blade or in the upper abdomen, loss of appetite, abdominal swelling, jaundice, fatigue, and fever.

For therapeutic and/or prophylactic applications, a composition comprising low dose carbon monoxide, is administered to the subject. In some embodiments, the low dose carbon monoxide is administered in an effective amount between about 150 ppm to about 750 ppm. In some embodiments, an effective amount of CO is about 150 ppm, about 155 ppm, about 160 ppm, about 165 ppm, about 170 ppm, about 175 ppm, about 180 ppm, about 185 ppm, about 190 ppm, about 195 ppm, about 200 ppm, about 205 ppm, about 210 ppm, about 215 ppm, about 220 ppm, about 225 ppm, about 230 ppm, about 235 ppm, about 240 ppm, about 245 ppm, about 250 ppm, about 255 ppm, about 260 ppm, about 265 ppm, about 270 ppm, about 275 ppm, about 280 ppm, about 285 ppm, about 290 ppm, about 295 ppm, about 300 ppm, about 305 ppm, about 310 ppm, about 315 ppm, about 320 ppm, about 325 ppm, about 330 ppm, about 335 ppm, about 340 ppm, about 345 ppm, about 350 ppm, about 355 ppm, about 360 ppm, about 365 ppm, about 370 ppm, about 375 ppm, about 380 ppm, about 385 ppm, about 390 ppm, about 395 ppm, about 300 ppm, about 305 ppm, about 310 ppm, about 315 ppm, about 320 ppm, about 325 ppm, about 330 ppm, about 335 ppm, about 340 ppm, about 345 ppm, about 350 ppm, about 355 ppm, about 360 ppm, about 365 ppm, about 370 ppm, about 375 ppm, about 380 ppm, about 385 ppm, about 390 ppm, about 395 ppm, about 400 ppm, about 405 ppm, about 410 ppm, about 415 ppm, about 420 ppm, about 425 ppm, about 430 ppm, about 435 ppm, about 440 ppm, about 445 ppm, about 450 ppm, about 455 ppm, about 460 ppm, about 465 ppm, about 470 ppm, about 475 ppm, about 480 ppm, about 485 ppm, about 490 ppm, about 495 ppm, about 500 ppm, about 505 ppm, about 510 ppm, about 515 ppm, about 520 ppm, about 525 ppm, about 530 ppm, about 535 ppm, about 540 ppm, about 545 ppm, about 550 ppm, about 555 ppm, about 560 ppm, about 565 ppm, about 570 ppm, about 575 ppm, about 580 ppm, about 585 ppm, about 590 ppm, about 595 ppm, about 600 ppm, about 605 ppm, about 610 ppm, about 615 ppm, about 620 ppm, about 625 ppm, about 630 ppm, about 635 ppm, about 640 ppm, about 645 ppm, about 650 ppm, about 655 ppm, about 660 ppm, about 665 ppm, about 670 ppm, about 675 ppm, about 680 ppm, about 685 ppm, about 690 ppm, about 695 ppm, about 700 ppm, about 705 ppm, about 710 ppm, about 715 ppm, about 720 ppm, about 725 ppm, about 730 ppm, about 735 ppm, about 740 ppm, about 745 ppm, or about 750 ppm. Values and ranges intermediate to the recited values are also contemplated as part of the present disclosure. In certain embodiments, an effective amount of CO is at a concentration of about 250 ppm.

In some embodiments, the low dose carbon monoxide is administered one, two, three, four, or five times per day. In some embodiments, the low dose carbon monoxide is administered more than five times per day. Additionally or alternatively, in some embodiments, the carbon monoxide is administered every day, every other day, every third day, every fourth day, every fifth day, or every sixth day. In some embodiments, the low dose carbon monoxide is administered weekly, bi-weekly, tri-weekly, or monthly. In some embodiments, the low dose carbon monoxide is administered for a period of one, two, three, four, or five weeks. In some embodiments, the low dose carbon monoxide is administered for six weeks or more. In some embodiments, the low dose carbon monoxide is administered for twelve weeks or more. In some embodiments, the low dose carbon monoxide is administered for a period of less than one year. In some embodiments, the low dose carbon monoxide is administered for a period of more than one year. In some embodiments, the low dose carbon monoxide is administered throughout the subject’s life.

In some embodiments, the carbon monoxide is administered daily for 1 week or more. In some embodiments of the methods of the present technology, the low dose carbon monoxide is administered daily for 2 weeks or more. In some embodiments of the methods of the present technology, the low dose carbon monoxide is administered daily for 3 weeks or more. In some embodiments of the methods of the present technology, the low dose carbon monoxide is administered daily for 4 weeks or more. In some embodiments of the methods of the present technology, the low dose carbon monoxide is administered daily for 6 weeks or more. In some embodiments of the methods of the present technology, the low dose carbon monoxide is administered daily for 12 weeks or more. In some embodiments, the low dose carbon monoxide is administered daily throughout the subject’s life. In some embodiments of the methods of the present technology, the low dose carbon monoxide is administered over a period of about 1 hour to about 24 hours. In certain embodiments, the low dose carbon monoxide is administered over a period of about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. Values and ranges intermediate to the recited values are also contemplated as part of the present disclosure.

### Determination of the Biological Effect of the Low Dose Carbon Monoxide

In various embodiments, suitable in vitro or in vivo assays are performed to determine the effect of a specific low dose carbon monoxide, and whether its administration is indicated for treatment. In various embodiments, in vitro assays can be performed with representative animal models, to determine if a given low dose carbon monoxide exerts the desired effect on reducing or eliminating signs and/or symptoms of metastatic cancer (e.g., a metastatic breast cancer). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### Carbon Monoxide Modes of Administration and Effective Dosages

Any method known to those in the art for contacting a cell, organ or tissue with low dose carbon monoxide may be employed. Suitable methods include in vitro, ex vivo, or in vivo methods. In vivo methods typically include the administration of low dose carbon monoxide to a mammal, suitably a human. When used in vivo for therapy, the low dose carbon monoxide is administered to the subject in effective amounts (i.e., amounts that have desired therapeutic effect). The dose and dosage regimen will depend upon the degree of the disease state of the subject, the characteristics of carbon monoxide formulation, e.g., its therapeutic index, and the subject’s history.

The effective amount may be determined during pre-clinical trials and clinical trials by methods familiar to physicians and clinicians. An effective amount of the low dose carbon monoxide may be administered to a mammal in need thereof by any of a number of well-known methods for administering pharmaceutical compounds. The low dose carbon monoxide may be administered systemically or locally.

The low dose carbon monoxide can be administered as pharmaceutical compositions, either singly or in combination, to a subject for the treatment or prevention of metastasis (e.g., lung metastasis). Such compositions typically include the active agent and a pharmaceutically acceptable carrier. As used herein, the term “pharmaceutically acceptable carrier” includes, but is not limited to, nanoparticles and adjuvants such as liposomes, biodegradable polylactic acid (“PLA”), biodegradable polyglycolic acid (“PGA”), and biodegradable poly(lactic-co-glycolic acid) (“PGLA”). Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

The pharmaceutical compositions comprising low dose carbon monoxide as disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent’s structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent’s structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED₅₀ with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of low dose carbon monoxide may be defined as a concentration of any value at or between about 150 ppm to about 500 ppm of CO at the target tissue. This concentration may be delivered by systemic doses or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, the low dose carbon monoxide may be combined with one or more additional therapies for the prevention or treatment of metastasis. Additional therapeutic agents include, but are not limited to, hormones (e.g., estrogen), chemotherapeutic agents, immunotherapeutic agents, surgery, radiation therapy, anti-angiogenic agents, non-steroidal anti-inflammatory drugs, or any combination thereof.

In some embodiments, the low dose carbon monoxide may be separately, sequentially or simultaneously administered with at least one additional therapeutic agent selected from the group consisting of alkylating agents, topoisomerase inhibitors, endoplasmic reticulum stress inducing agents, antimetabolites, immunotherapeutic agents, mitotic inhibitors, nitrogen mustards, nitrosoureas, alkylsulfonates, platinum agents, taxanes, vinca agents, anti-estrogen drugs (drugs that prevent estrogens from mediating their biological effects, including but not limited to, selective estrogen receptor modulators (SERMs) like tamoxifen, clomifene, and raloxifene, the ER silent antagonist and selective estrogen receptor degrader (SERD) fulvestrant, aromatase inhibitors (AIs) like anastrozole, and antigonadotropins, androgens/anabolic steroids, progestogens, and GnRH analogs), aromatase inhibitors, ovarian suppression agents, VEGF/VEGFR inhibitors, EGF/EGFR inhibitors, PARP inhibitors, cytostatic alkaloids, cytotoxic antibiotics, including protein synthesis inhibitors, endocrine/hormonal agents, bisphosphonate therapy agents, phenphormin, anti-angiogenic agents, Histone deacetylase inhibitors, non-steroidal anti-inflammatory drugs (NSAIDs), and targeted biological therapy agents (e.g., therapeutic peptides described in US 6306832, WO 2012007137, WO 2005000889, WO 2010096603 etc.).

Additionally or alternatively, in any of the embodiments of the methods disclosed herein, the additional therapeutic agent is a chemotherapeutic agent selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, ABRAXANE^{®} (albumin-bound paclitaxel), protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, alpelisib, and mixtures thereof.

Additionally or alternatively, in some embodiments, the additional therapeutic agent is an antimetabolite selected from the group consisting of 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Additionally or alternatively, in some embodiments, the additional therapeutic agent is a taxane selected from the group consisting of accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Additionally or alternatively, in some embodiments, the additional therapeutic agent is a DNA alkylating agent selected from the group consisting of cyclophosphamide, chlorambucil, melphalan, bendamustine, uramustine, estramustine, carmustine, lomustine, nimustine, ranimustine, streptozotocin; busulfan, mannosulfan, and mixtures thereof.

Additionally or alternatively, in some embodiments, the additional therapeutic agent is a topoisomerase I inhibitor selected from the group consisting of SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof.

Additionally or alternatively, in some embodiments, the additional therapeutic agent is a topoisomerase II inhibitor selected from the group consisting of amsacrine, etoposide, etoposide phosphate, teniposide, daunorubicin, mitoxantrone, ellipticines, aurintricarboxylic acid, doxorubicin, and HU-331 and combinations thereof.

Additionally or alternatively, in some embodiments, the additional therapeutic agent is an immunotherapeutic agent selected from the group consisting of immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

Additionally or alternatively, in some embodiments, the additional therapeutic agent is an anti-angiogenic agent selected from the group consisting of bevacizumab, cediranib, axitinib, anginex, sunitinib, sorafenib, pazopanib, vatalanib, cabozantinib, ponatinib, lenvatinib, SU6668, Everolimus (Afinitor^{®}), Lenalidomide (Revlimid^{®}), Ramucirumab (Cyramza^{®}), Regorafenib (Stivarga^{®}), Thalidomide (Synovir, Thalomid^{®}), Vandetanib (Caprelsa^{®}), and Ziv-aflibercept (Zaltrap^{®}).

Additionally or alternatively, in some embodiments, the additional therapeutic agent is a Histone deacetylase inhibitor selected from the group consisting of trichostatin A (TSA), tubacin, apicidin, depsipeptide, MS275, BML-210, RGFP966, MGCD0103, LBH589, splitomicin, FK228, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC202, Mocetinostat, Romidepsin, Nicotinamide, Sirtinol, Cambinol, and EX-527.

Examples of NSAIDs include indomethacin, fenoprofen, ibuprofen, flufenamic acid, aspirin, celecoxib, diclofenac, diflunisal, etodolac, ketoprofen, ketorolac, nabumetone, naproxen, oxaprozin, piroxicam, salsalate, sulindac, and tolmetin.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above.

### Example 1: Experimental Materials and Methods

Methods summary. In vitro transwell migration assays and breast cancer and pancreatic cancer xenograft mouse models were used to determine the efficacy of low-dose CO for inhibiting cancer cell migration and metastasis. In vivo experiments were randomized, unblinded, and conducted in accordance with the National Institutes of Health Guide for the Care and Use of Laboratory Animals. RNA-Seq data was aligned to the hg19 reference genome using TopHat2 and transcriptome reconstruction was performed by Cufflinks. DEGs were generated by DESeq2.

### Cell Culture, Transfection and Treatment

MDA-MB-231/TGL cell lines was obtained from Dr. Joan Massagué (Minn, et al, J. Clin. Invest., 115:(1), 44-55 (2005)). CM cell line was obtained from Dr. Paolo Pozzilli (S. Kobayashi et al., Oncogenesis 8, 16 (2019)). MCF7 breast cancer cells and 8988T pancreatic cancer cells were infected with viruses carrying thymidine kinase/green fluorescent protein/luciferase fusion reporter (TGL), which was obtained from Drs. Inna Serganova and Ronald Blasberg (V. Ponomarev et al., European journal of nuclear medicine and molecular imaging 31, 740-751 (2004)). GFP-positive MCF7/TGL and 8999T/TGL cells were enriched by fluorescence-activated cell sorting. H1975, lung adenocarcinoma cell line, was obtained from ATCC (Manassas, VA). MCF7/TGL/pQ and MCF7/TGL/HA-Bcl-xL cell lines were generated by infecting MCF7/TGL cells with viruses carrying pQXCIP or pQXCIP-HA-Bcl-xL, and selected in the medium containing 0.5-1 µg/ml puromycin. Human CYP1B1cDNA (GeneScript) was transiently transfected into MCF7/TGL, MDA-MB-231/TGL, and 8988T/TGL with Lipofectamine 3000 according to the manufacture’s protocol (Invitrogen, Carlsbad, CA). Cells were treated with CO at 250 ppm for 16 hours. Control air condition was a regular cell culture incubator with 5% CO₂. Cells were also treated with 30 µM Hemin (HY-19424, MedChemExpress, Monmouth Junction, NJ) in standard tissue culture incubator or 250 ppm CO.

### In Vitro Transwell Migration Assay

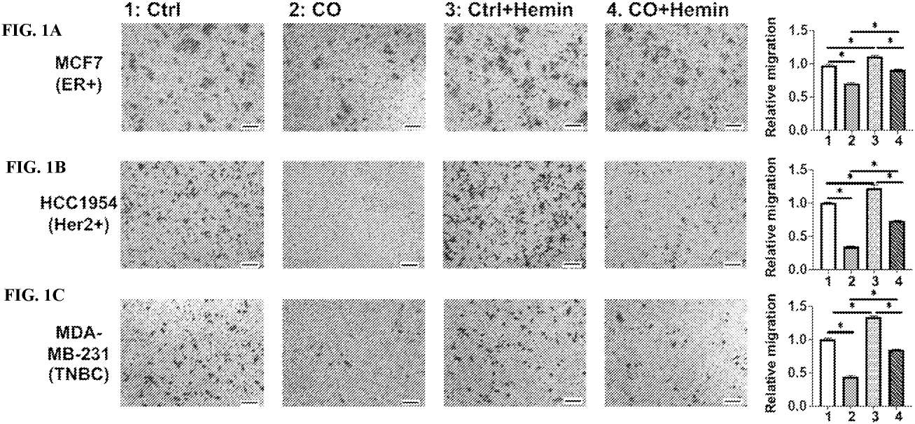

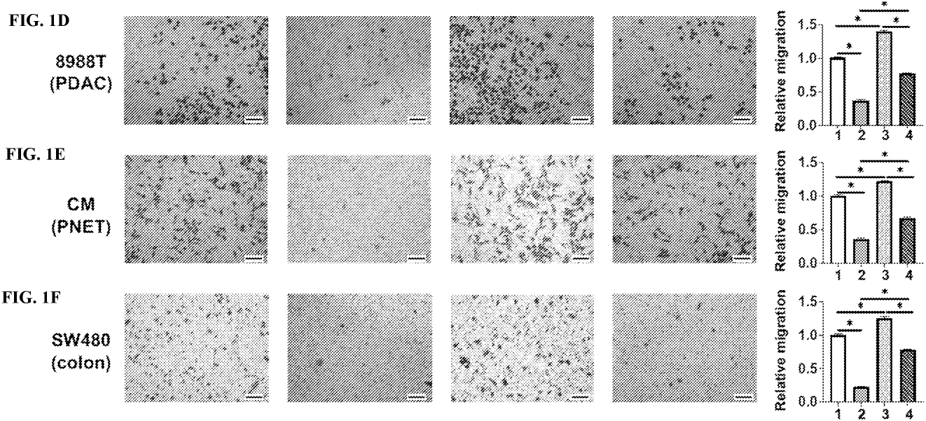

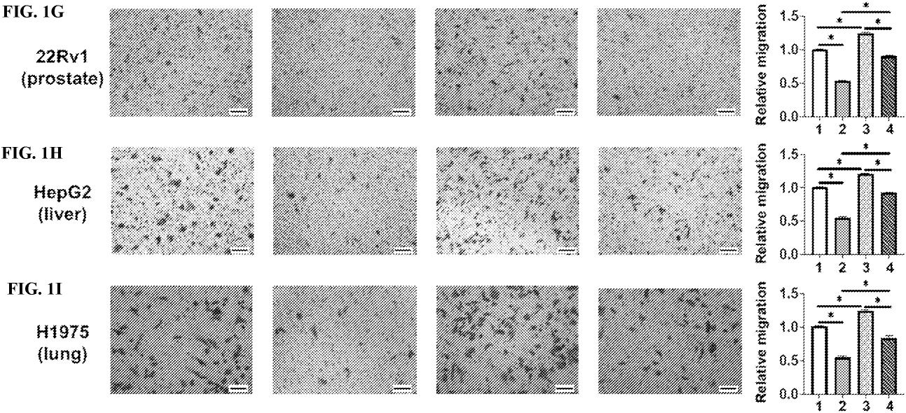

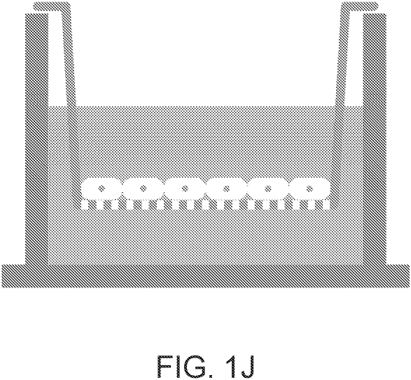

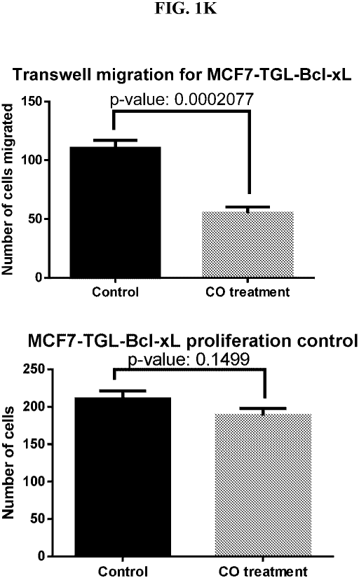

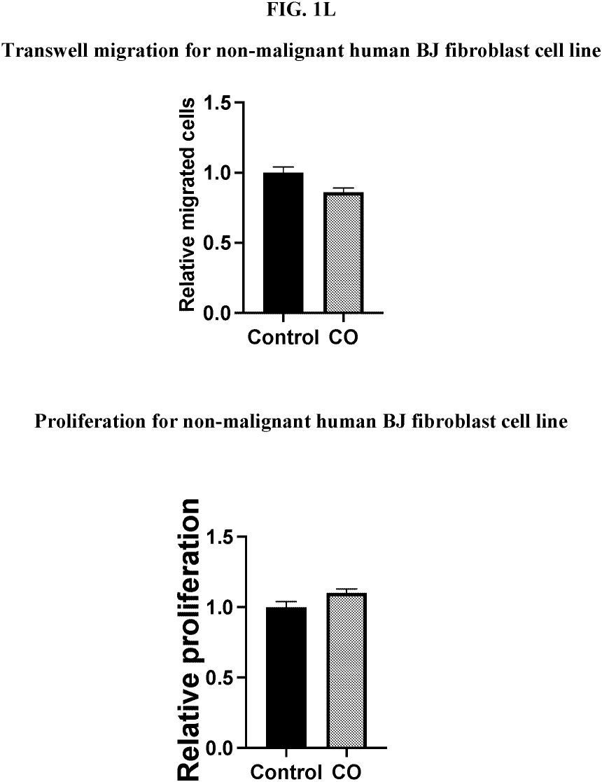

6.5 mm Transwell with 8.0 µm Pore Polycarbonate Membrane Insert (Corning, NY, Cat. No. 3422) was used for the cell migration assay (FIG. 1J). After 16 hours of incubation, cells migrated to the opposite side of the upper chambers were fixed with 4% paraformaldehyde, stained with 0.2% crystal violet for 30 min and counted in five fields under × 20 magnification. In parallel, cells were seeded on 24-well plates for proliferation assay. Cells for proliferation control were fixed with 4% paraformaldehyde, stained with 0.2% crystal violet for 30 min and lysed with methanol to release the crystal violet as described by M. Feoktistova, P. Geserick, & M. Leverkus, Cold Spring Harb Protoc 2016, pdb prot087379 (2016). The optical density was measured at 595 nm (OD595). The cell numbers, medium, and serum gradient for different cell lines used were listed in Table 1. To supplement heme/hemin in the transwell migration assay, 30 µM hemin (MedChemExpress, Monmouth Junction, NJ, Cat. No. HY-19424) was added to both the cell suspension in the upper chambers and the bottom wells.

TABLE 1

| Cell line | medium | Number of cells used for Transwell | Serum gradient for Transwell | Number of cells used for proliferation |
|---|---|---|---|---|
| MCF7 | DMEM | 2 × 10⁵ | 1%-10% | 5 × 10⁴ |
| HCC1954 | RPMI | 5 × 10⁵ | 1%-10% | 5 × 10⁵ |
| MDA-MB-231 | DMEM | 5 × 10⁴ | 1%-10% | 5 × 10⁴ |
| 8988T | DMEM | 1 × 10⁵ | 0%-10% | 5 × 10⁴ |
| CM | DMEM | 5 × 10⁴ | 0%-20% | 5 × 10⁴ |
| SW480 | DMEM | 1 × 10⁶ | 0%-20% | 1 × 10⁵ |
| 22Rv1 | RPMI | 5 × 10⁵ | 1%-10% | 1 × 10⁵ |
| HepG2 | MEM | 5 × 10⁵ | 1%-10% | 1 × 10⁵ |
| H1975 | RPMI | 2 × 10⁵ | 0%-10% | 2 × 10⁵ |

### Metabolomic Assay

7 × 10⁶ cells were seeded in 10-cm cell culture plates with DMEM containing 10% FBS, 0.2 mM L-glutamine and 1% penicillin/streptomycin. Additionally, MCF7/TGL/pQ was cultured in media containing 0.5 µgml⁻¹ puromycin and 25 mM HEPES. After 16 hours of incubation, metabolites were extracted from the cells using 80% methanol. The remaining steps were carried out by Weill Cornell Medicine Proteomics and Metabolomics Core Facility according to M. D. Goncalves et al., Proc Natl Acad Sci USA 115, E743-E752 (2018). All data analyses were done using MetaboAnalyst 4.0 (www.metaboanalyst.ca/).

### Heme Assay

Cells were grown to 75-80% confluence in 10-cm cell culture plates and collected in 1 ml cold PBS by cell scraper. 10 µl of the cells was transferred to a new tube for protein lysate preparation and protein concentration measurement. The rest of the cells were lysed in Heme extraction buffer (acetone: HCl: water = 25: 1.3: 5) as described by J. Hooda, M. Alam, & L. Zhang, J Vis Exp, e51579 (2015). Heme levels were measured using a heme colorimetric assay kit (BioVision, Milpitas, CA, Cat. No. K672-100) and normalized by the protein concentration of each sample.

### Western Blot Analysis

1.5 × 10⁶ cells were seeded in 6-cm cell culture plates with DMEM containing 10% FBS, 0.2 mM L-glutamine and 1% penicillin/streptomycin. Additionally, MCF7/TGL/pQ was cultured in media containing 0.5 µgml⁻¹ puromycin and 25 mM HEPES. After 16 hours of incubation, cells were lysed with RIPA buffer (0.1% SDS, 1% TritonX-100, 0.5% sodium deoxycholate, 25 mM Tris pH 8.0, 150 mM NaCl, and 1 mM EDTA) supplemented with a protease inhibitor mixture and PhosSTOP (Roche, Basel, Switzerland). Proteins were quantified with Bradford assay (Bio-Rad, Hercules CA). Equal amounts of proteins were separated with SDS-PAGE and transferred to nitrocellulose membranes. To visualize equal protein loading, blots were stained with Ponceau S. Blots were incubated in 5% non-fat milk in TBST, probed with primary antibodies to CYP1B1 (1:1,000, Abcam, Cambridge, United Kingdom, Cat. No. 32649), HO-1 (1:1,000, Santa Cruz Biotechnology, Dallas, TX, Cat. No. 136960), BACH1 (1:1,000, ABclonal, Woburn, MA, Cat. No. A5393), E-cadherin (1:1,1000, BD Biosciences (Cat. No. 610181), San Jose, CA), α-tubulin (1:1,000, Sigma (Cat. No. T5168), St. Louis MO), and vimentin (1:1,000, Cell Signaling Tech (Cat. No. 5741), Danvers, MA), and then were incubated with horseradish peroxidase-conjugated secondary antibodies. Protein bands were visualized by enhanced chemical luminescence (Pierce, Dallas, TX).

### Mouse Models of Metastasis

NSG mice were generated by the Jackson Laboratory. All mice were housed in accordance with the institutional guidelines. All procedures involving mice were approved by the institutional animal care and use committee of Weill Cornell Medicine. 1 × 10⁵ or 5 × 10⁴ MDA-MB-231/TGL cells in 150 µl PBS were injected into the tail veins of female NSG mice at the age of 7-8 weeks (n=5/group for 1× 10⁵ cells and n = 6/group for 5 × 10⁴ cells). 1× 10⁴ 8988T/TGL cells in 100 µl PBS were injected into spleen of NSG mice at age of 7-8 weeks (n=4/group, male and female) to observe the liver metastasis (G. Zhang, & Y. N. Du, Methods Mol Biol 1882, 309-320 (2019)). The spleen was removed after the injection. Starting one day after injection, mice were randomized into two groups. One group was kept in a regular mouse holding room (control), and the other group was put into a chamber delivering 250 ppm CO 3 hours daily. Mice were subjected to bioluminescent imaging using the In Vivo Imaging System Spectrum (PerkinElmer, Waltham, MA) at 0, 1, 3 days and then weekly after tumor cells were injected until the final time points, as described previously by Y. C. Du, D. S. Klimstra, & H. Varmus, PLoS One 4, e6932 (2009)). Luciferase signals were transformed to nature log scale before analysis. GEE method was used to test the overall difference of tumor growth over time. All analyses were performed in statistical software SAS Version 9.4 (SAS Institute, Cary, NC).

### Tissue Preparation and Immunohistochemical Analysis

Mouse tissues were removed and fixed in 10% buffered formalin overnight at room temperature. Formalin-fixed/paraffin-embedded sections (5-µm) were deparaffinized and rehydrated by passage through a graded xylene/ethanol series before staining. Immunochemistry was performed using the VECTASTAIN Elite ABC kit following the manufacturer’s instructions. Tumor number and tumor size were quantified by scanning the whole tissue slide of each mouse under microscope. The size of tumor area of each tumor was measured by the cellSens imaging software (Olympus, Tokyo, Japan). Total tumor area of each mouse or total tumor number of each mouse was plotted. The primary antibodies used were cleaved caspase 3 (1:333; Cell Signaling Tech (Cat. No. 9661), Danvers, MA) and Ki67 (1:1000, Vector Labs (Cat. No. VP-K451), Burlingame, CA).

### Quantitative Real-Time Reverse Transcription PCR

mRNA was isolated from cells (MCF7/TGL and MDA-MB-231/TGL) grown on 6-cm dishes using RNeasy mini kit (Qiagen, Hilden, Germany) containing gDNA Eliminator spin columns. cDNA was generated using the SuperScript III First-strand synthesis system with random hexamers (Invitrogen, Carlsbad, CA), and power SYBR green (Invitrogen, Carlsbad, CA)-based quantitative real-time PCR was performed using primer specific for HRG1 (human, forward: 5′- TCACATTGCAGTATTCGTGTGC -3′ (SEQ ID NO: 1), reverse: 5′- CATCCCGTCGCCTTTTATTGA -3′ (SEQ ID NO: 2)) or HCP1 (human forward: 5′- AGAGCTGGACAATGGATCGGT -3′ (SEQ ID NO: 3), reverse: 5′-GCCTTGCTGATAGCCATGACTC -3′ (SEQ ID NO: 4)), CYP1B1 (human, forward: 5′TGAGTGCCGTGTGTTTCGG -3′ (SEQ ID NO: 5), reverse: 5′-GTTGCTGAAGTTGCGGTTGAG -3′ (SEQ ID NO: 6)), SP1 (human, forward: 5′-GTGGCCGCTACCTTCACTG -3′ (SEQ ID NO: 7), reverse: 5′-GCCCCACTCCTACTTGGTC -3′ (SEQ ID NO: 8)) with the comparative CT method (ΔΔCT; ABI).

### RNA-Seq Analysis

Total RNA was extracted using the RNeasy Plus mini kit (Cat. No. 74134; Qiagen, Hilden, Germany) and QIAshredder kit (Cat. No. 79654; Qiagen, Hilden, Germany), according to the manufacturer’s protocol. Following RNA isolation, total RNA integrity was checked using a 2100 Bioanalyzer (Agilent Technologies, Santa Clara, CA). RNA concentrations were measured using the NanoDrop system (Thermo Fisher Scientific, Inc., Waltham, MA). Preparation of RNA sample library and RNA-seq were performed by the Genomics Core Laboratory at Weill Cornell Medicine. Messenger RNA was prepared using TruSeq Stranded mRNA Sample Library Preparation kit (Illumina, San Diego, CA), according to the manufacturer’s instructions. The normalized cDNA libraries were pooled and sequenced on Illumina HiSeq4000 sequencer with pair-end 50 cycles. cDNA libraries were generated using the Illumina TruSeq RNA Sample preparation kit and sequenced with paired-end 50 bps on HiSeq4000 sequencer. The raw sequencing reads in BCL format were processed through bcl2fastq 2.19 (Illumina, San Diego, CA) for FASTQ conversion and demultiplexing. RNA reads were aligned and mapped to the hg19 human reference genome by TopHat2 (Version2.0.11) (ccb.jhu.edu/software/tophat/index.shtml) (D. Kim et al., Genome biology 14, R36 (2013)), and transcriptome reconstruction was performed by Cufflinks (Version 2.1.1) (cole-trapnell-lab.github.io/cufflinks/). The abundance of transcripts was measured with Cufflinks in Fragments Per Kilobase of exon model per Million mapped reads (FPKM) (C. Trapnell et al., Nat Biotechnol 31, 46-53 (2013); C. Trapnell et al., Nat Biotechnol 28, 511-515 (2010)). Gene expression profiles were constructed for differential expression, cluster, and principal component analyses with the DESeq2 package (bioconductor.org/packages/release/bioc/html/DESeq2.html) (M. I. Love, W. Huber, & S. Anders, Genome biology 15, 550 (2014)). For differential expression analysis, pairwise comparisons between two or more groups using parametric tests where read-counts follow a negative binomial distribution with a gene-specific dispersion parameter. Corrected p-values were calculated based on the Benjamini-Hochberg method to adjusted for multiple testing.

### Example 2: Effects of Low Dose CO in Various Cancer Cell Lines Using in vitro Transwell Migration Assay

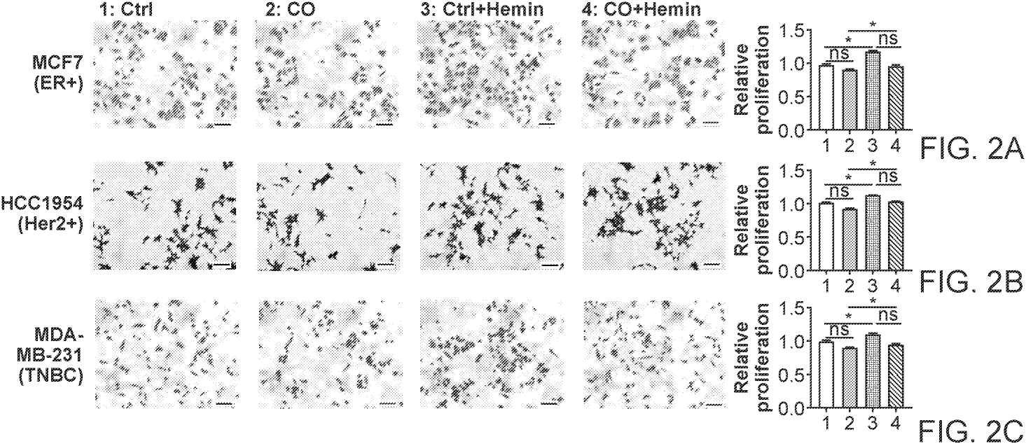

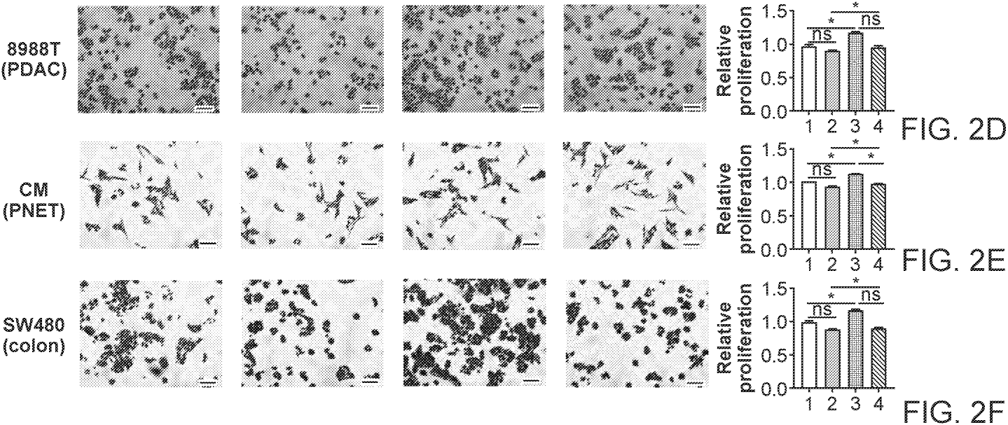

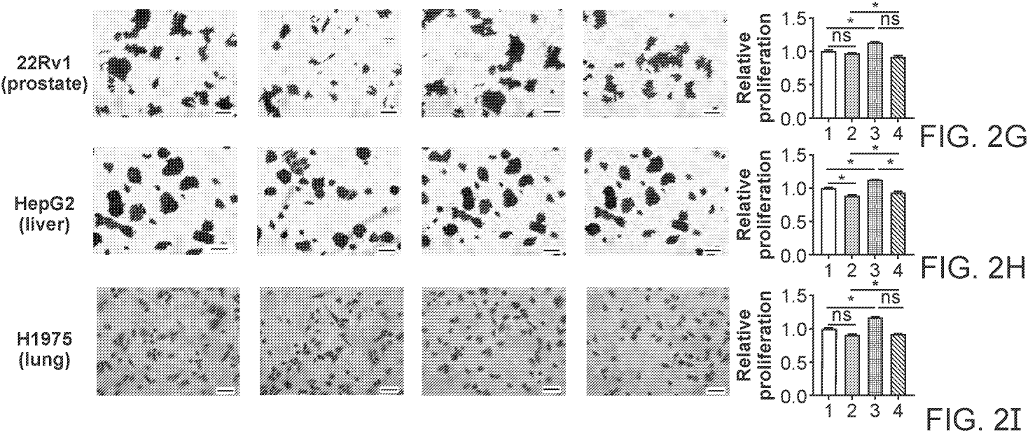

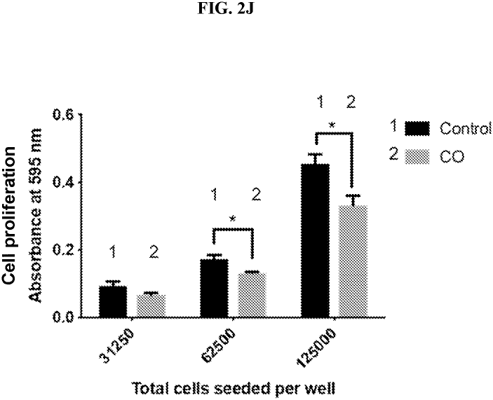

To examine whether CO has an effect on tumor cell migration, in vitro transwell migration assays were performed. Various human cancer cell lines, including 3 types of breast cancer (ER⁺: MCF7 cells, HER2⁺: HCC1954 cells, triple-negative breast cancer (TNBC): MDA-MB-231 cells), pancreatic ductal adenocarcinoma (PDAC) (8988T cells), pancreatic neuroendocrine tumor (PNET) (CM cells), colon cancer (SW480 cells), prostate cancer (22Rv1 cells), liver cancer (HepG2 cells), and lung cancer (H1975 cells) were seeded in the upper chamber of 8-µm porous polycarbonate membranes. Cell migration through the transwell membrane along a serum gradient (Table 1) after 16 hours in the presence or absence of 250 ppm CO were measured. It was found that 250 ppm CO significantly reduced migration of all these cancer cell lines (FIGS. 1A-1I, and 1K). This low-dose of CO for 16 hours did not affect cell proliferation (FIGS. 2A-2I and 1K), suggesting that the reduction in migration under low-dose CO was not due to cytotoxicity.

5 × 10⁴ MDA-MB-231-TGL cells were seeded in the upper chamber of 8-µm porous polycarbonate membranes with DMEM containing 0% FBS, 0.2 mM L-glutamine and 1% penicillin/streptomycin. The lower chambers were filled with DMEM containing 10% FBS, 0.2 mM L-glutamine and 1% penicillin/streptomycin. 3 × 10⁴, 6 × 10⁴, or 1.2 × 10⁵ MDA-MB-231-TGL cells were seeded in a 24-well plate with DMEM containing 10% FBS, 0.2 mM L-glutamine and 1% penicillin/streptomycin as proliferation control. After 16 hours of incubation, cells migrating to the opposite side of the upper chambers of the inserts and cells for proliferation control were fixed with 4% paraformaldehyde, stained with 0.1% crystal violet for 30 min and lysed with methanol to release the crystal violet. The optical density was measured at 595 nm (OD595). FIG. 2J shows that CO treatment had greater inhibitory effect on MDA-MB-231 cell proliferation when cells were seeded at higher density (6.25 × 10⁴ or 12.5 × 10⁴ cells per 24-well) than at lower density (3.125 × 10⁴ cells per 24-well).

Accordingly, compositions including low doses of carbon monoxide as disclosed herein are useful in methods for treating or preventing metastasis in a subject in need thereof.

### Example 3: Effects of Low Dose CO in Lung Metastasis in a Mouse Model of Human Breast Cancer Xenograft

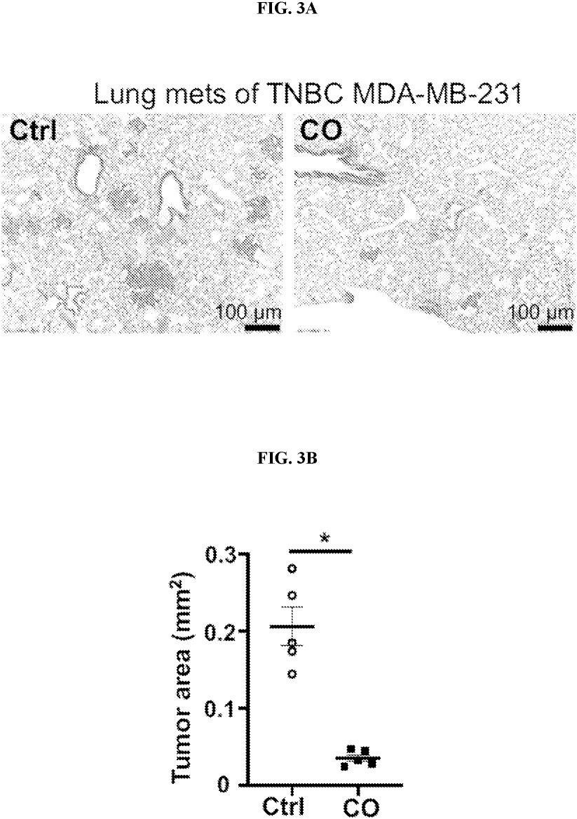

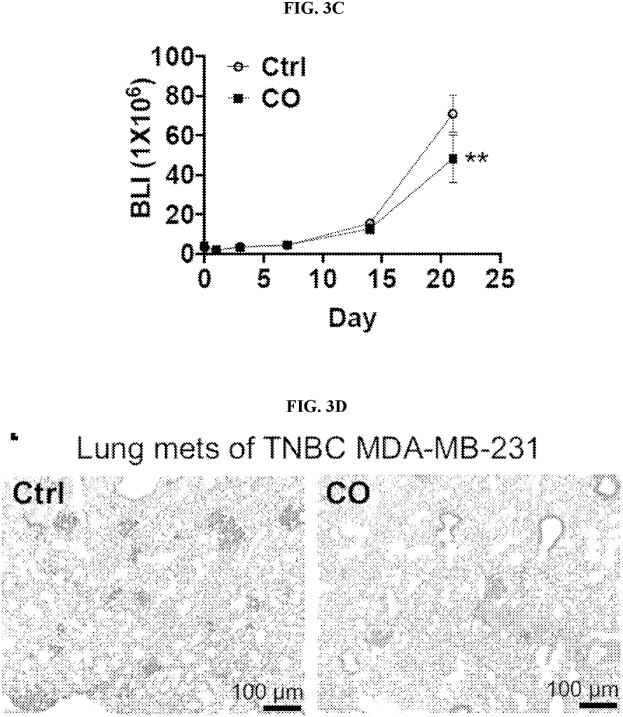

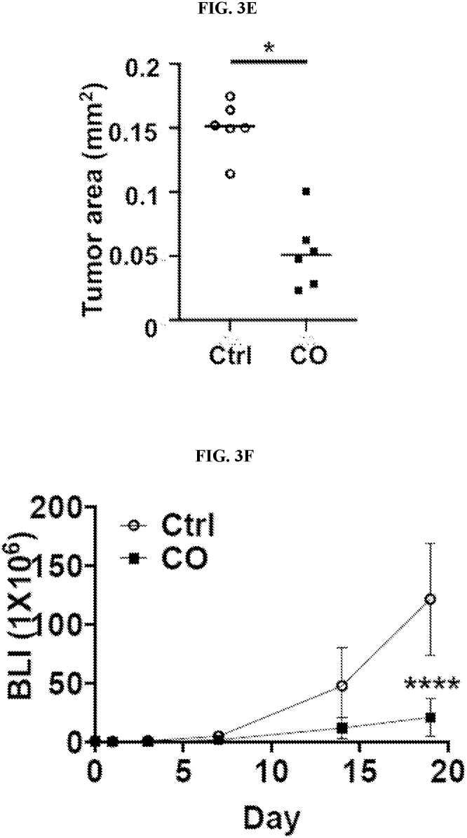

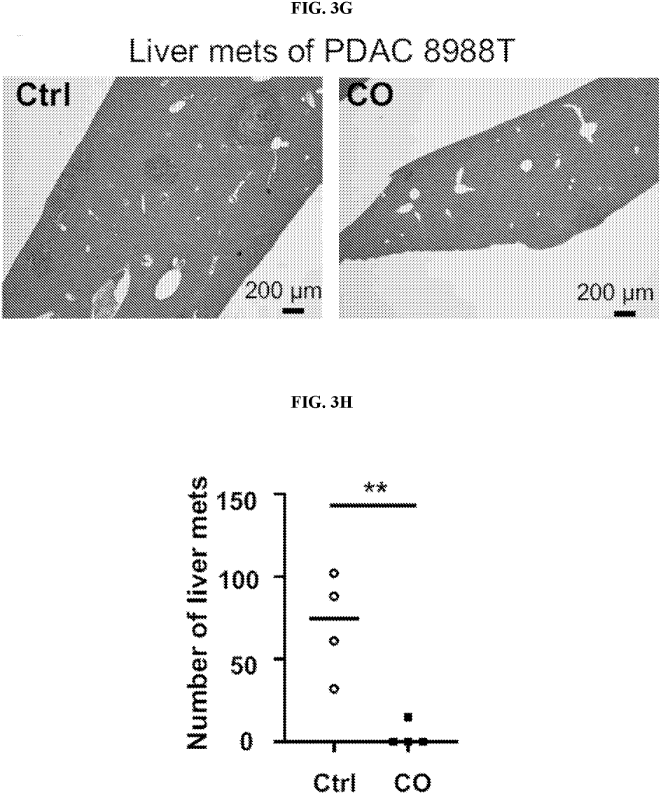

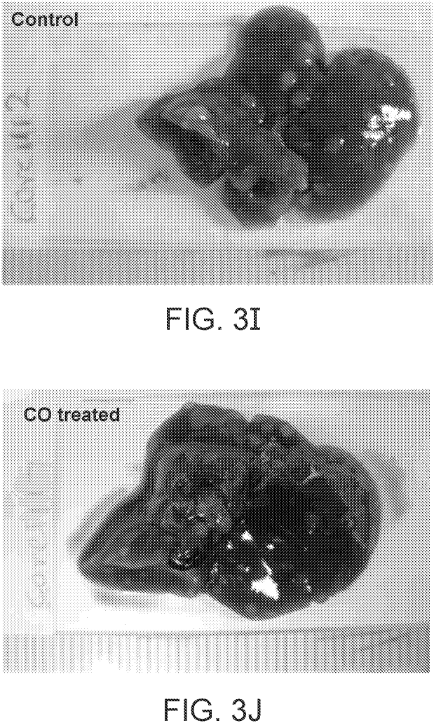

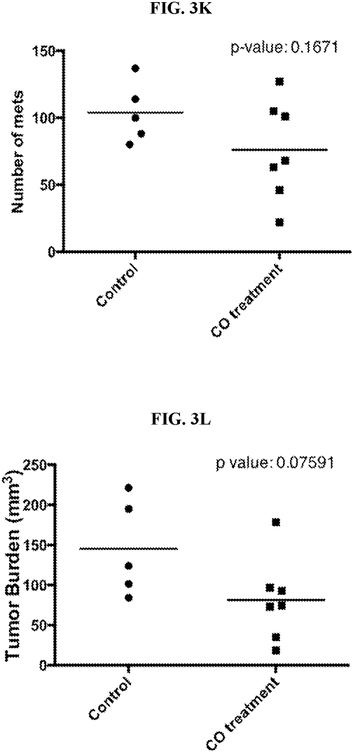

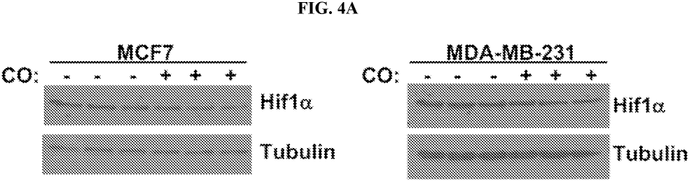

To determine whether 250 ppm CO inhibits cancer metastasis in vivo, an experimental tail vein metastasis assay with TNBC MDA-MB-231 cell line was performed. 0.1 million MDA-MB-231/TGL (luciferase reporter) cells were injected into the tail vein of the NOD/scid-lL2Rgc knockout (NSG) immunodeficient mice. Mice were randomly divided in to two groups after injection. One group was kept in regular mouse holding room (control), and the other group of mice were put into a chamber delivering 250 ppm CO gas 3 hours daily starting the day after injection of tumor cells. The control group became lethargy after 23 days and needed to be euthanized. In contrast, 250 ppm CO-treated mice looked healthy and had no signs of sickness throughout the experiment. Histological analysis of the lung sections at the 23-day time point showed significantly lower metastatic tumor burden in the CO treated group (FIGS. 3A-3B).

Because luciferase signals from mice injected with 0.1 million MDA-MB-231/TGL cells were saturated for bioluminescent imaging, a similar experiment was conducted using 5 × 10⁴ MDA-MB-231/TGL cells. Mice were randomly divided in to two groups after injection. One group was kept in regular mouse holding room (control), and the other group of mice were put into a chamber delivering 250 ppm CO gas 3 hours daily, 7 days/week starting the day after injection of tumor cells. Mice were subjected to in vivo bioluminescent imaging at 0, 1, 3 days, and 1, 2, and 3 weeks after tumor cells were injected. The bioluminescent signals from CO treated group were significantly lower than those from the control group (FIG. 3C, P = 0.0016, GEE method), suggesting that low-dose of CO inhibited the outgrowth of micro-metastases in the lung. Histological analysis of the lung sections also showed significantly lower metastatic tumor burden in the CO-treated group compared to the untreated control group 25 days after the injection (FIGS. 3D-3E).

Accordingly, compositions including low doses of carbon monoxide as disclosed herein are useful in methods for treating or preventing metastasis in a subject in need thereof.

### Example 4: Effects of Low Dose CO in Liver Metastasis in a Mouse Model of Human Pancreatic Cancer Xenograft

Because CO was administrated as its gaseous form through the respiratory system, it was unclear whether it would be effective to halt metastasis in organs other than the lung. To investigate this, an orthotopic model of liver metastasis of PDAC was employed (G. Zhang, & Y. N. Du, Methods Mol Biol 1882, 309-320 (2019)). PDAC 8988T cells were engineered with luciferase reporter (8988T/TGL) to follow the tumor cells inside the recipient mice by in vivo bioluminescence imaging. 5 × 10⁴ 8988T/TGL cells were intrasplenically injected into NSG mice. Mice were randomly divided in to two groups after injection. One group was kept in regular mouse holding room (control), and the other group of mice were put into a chamber delivering 250 ppm CO gas 3 hours daily, 5 days a week, starting one day after injection. Significant difference in bioluminescence over the time course was detected between control and CO group (P < 0.0001, GEE method) (FIG. 3F). Histological analysis of the liver sections 19 days after the injection showed significantly less metastatic tumor numbers in the CO-treated group (FIGS. 3G-3H). The data suggested that 250 ppm inhaled CO was effective to inhibit liver metastasis of PDAC.

In another experiment, immunodeficient NOD/scid-IlL2Rgc knockout mice were injected with 1.5 × 10⁶ N134/RHAMM^{B} cells in 120 µl PBS via tail veins. Tumors developed 24 days after injection with 1.5 × 10⁶ N134/RHAMM^{B} cells, and livers were assessed. FIGS. 3I-3L demonstrate that carbon monoxide treatment reduced liver metastases in immunodeficient NOD/scid-IlL2Rgc knockout (NSG) mice with tumors compared with untreated controls.

Accordingly, compositions including low doses of carbon monoxide as disclosed herein are useful in methods for treating or preventing metastasis in a subject in need thereof.

### Example 5: Low-Dose CO Reduces Intracellular Heme Levels to Suppress Migration

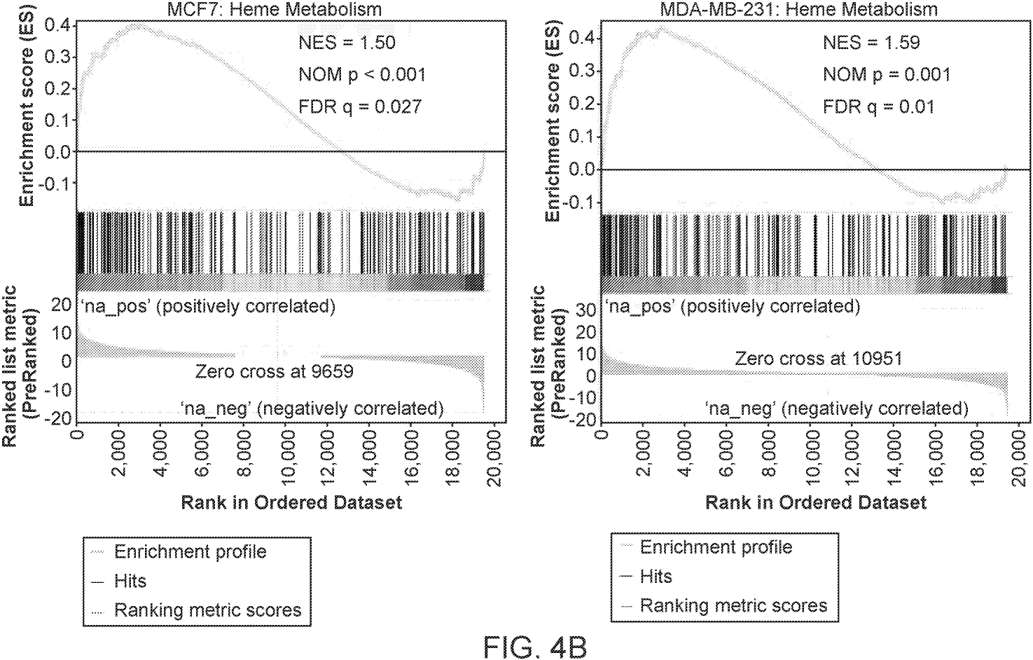

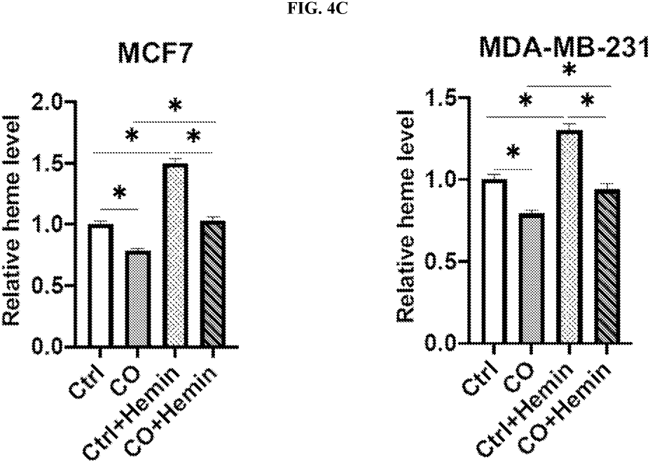

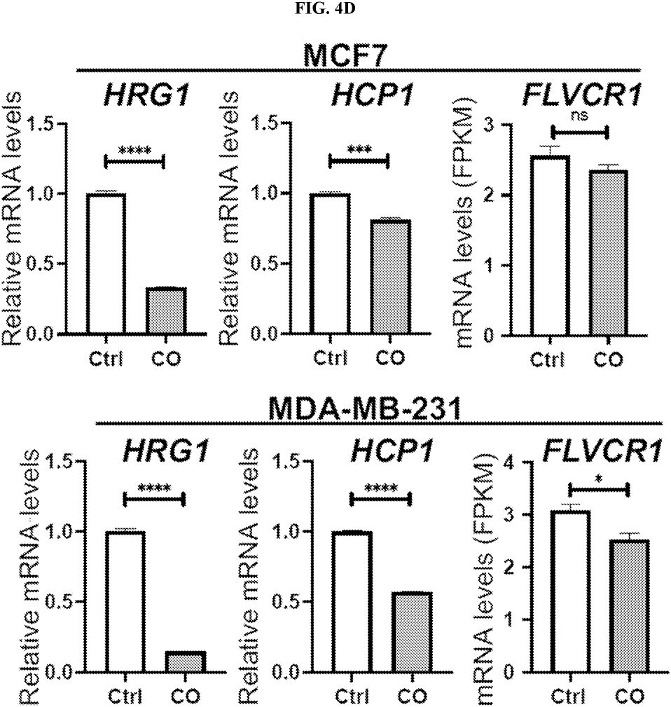

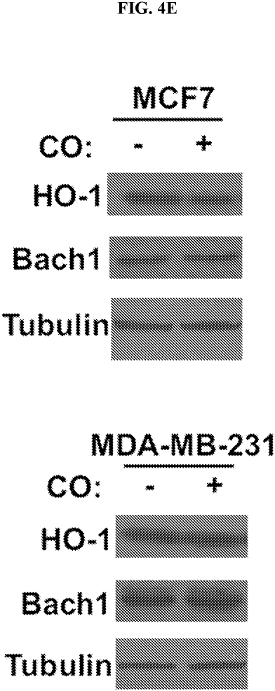

To investigate whether 250 ppm CO induces hypoxia, HiF1α protein levels in low-dose CO-treated MCF7/TGL and MDA-MB-231/TGL cells were examined. Compared to the control cells grown in a standard tissue culture incubator, HiF1α protein levels were not changed after 250 ppm CO treatment for 16 hours (FIG. 4A). To identify genes and pathways that were affected by 250 ppm CO, RNA-Seq and gene set enrichment analysis (GESA) of MCF7 and MDA-MB-231 cells were performed after 16 hours in a CO incubator or in a regular cell culture incubator. Transcriptome analysis revealed that low-dose CO significantly affected the expression of 3,915 genes in MCF7 and the expression of 4,655 genes in MDA-MB-231 (the adjusted P value <0.01, GEO accession # GSE173986, www.ncbi.nlm.nih.gov/geo/query/acc.cgi?acc=GSE173986). When analyzing the results using the hallmark gene sets that cover well-defined biological states and processes in the Molecular Signature Database (Broad Institute), it was found that heme metabolism was positively enriched in the 2 breast cancer cell lines treated by 250 ppm CO (FIG. 4B; overlapping gene sets regulated by low-does CO in both MCF7 and MDA-MD-231 is shown in Table 2). Heme is an essential iron-containing molecule, and it serves as a co-factor in proteins involved in fundamental biological processes. To assess the intracellular heme levels affected by low-dose CO treatment, oxidized heme levels in the form of hemin (Fe³⁺ state) were measured because free heme (Fe²⁺ state) is readily oxidized and cannot be measured. It was found that CO significantly reduced heme/hemin levels in MCF7 and MDA-MB-231 cells (FIG. 4C). To determine whether supplementing heme/hemin in the culture medium can negate the anti-migration effect of CO on cancer cells, 30 µM hemin was added into the medium in both the upper and lower chambers for transwell migration assays. It was found that 30 µM hemin treatment increased intracellular heme levels and partially restored cell migration inhibited by CO (FIGS. 1C-1E) while it slightly increased cell proliferation of various cancer cell lines (FIGS. 2C-2E).

TABLE 2

Overlapping gene sets regulated by low-does CO in both MCF7 and MDA-MD-231 with nominal p-value <5% and FDR < 25%.

| Upregulated gene sets by 250 ppm CO | Size | Cell line | ES | NES | NOM p | FDR q |
|---|---|---|---|---|---|---|
| HALLMARK_HEME_METABOLISM | 181 | MCF7 MDA-MB-231 | 0.4 0.4 | 1.50 1.59 | < 0.001 0.001 | 0.027 0.012 |
| HALLMARK_TNFA_SIGNALING_VIA_NFKB | 192 | MCF7 MDA-MB-231 | 0.5 0.5 | 1.96 1.97 | < 0.001 < 0.001 | < 0.001 < 0.001 |
| HALLMARK_KRAS_SIGNALING_UP | 186 | MCF7 MDA-MB-231 | 0.5 0.4 | 1.86 1.41 | < 0.001 0.015 | 0.001 0.042 |
| HALLMARK_XENOBIOTIC_METABOLISM | 189 | MCF7 MDA-MB-231 | 0.4 0.4 | 1.46 1.56 | < 0.001 0.001 | 0.031 0.016 |
| HALLMARK_INFLAMMATORY_RESPONSE | 188 | MCF7 MDA-MB-231 | 0.4 0.5 | 1.42 1.64 | 0.004 < 0.001 | 0.040 0.008 |
| HALLMARK_P53_PATHWAY | 192 | MCF7 MDA-MB-231 | 0.3 0.5 | 1.25 1.66 | 0.041 < 0.001 | 0.153 0.009 |
| HALLMARK_IL6_JAK_STAT3_SIGNALING | 78 | MCF7 MDA-MB-231 | 0.4 0.4 | 1.44 1.44 | 0.015 0.028 | 0.037 0.033 |

| Downregulated gene sets by 250 ppm CO | Size | Cell line | ES | NES | NOM p | FDR q |
|---|---|---|---|---|---|---|
| HALLMARK_MYC_TARGETS_V1 | 196 | MCF7 MDA-MB-231 | -0.51 -0.54 | -2.00 -2.17 | < 0.001 < 0.001 | < 0.001 < 0.001 |
| HALLMARK_MYC_TARGETS_V2 | 58 | MCF7 MDA-MB-231 | -0.58 -0.7 | -1.92 -2.37 | < 0.001 < 0.001 | < 0.001 < 0.001 |
| HALLMARK_E2F_TARGETS | 196 | MCF7 | -0.4 | -1.58 | < 0.001 | 0.020 |
| | | MDA-MB-231 | -0.53 | -2.10 | < 0.001 | < 0.001 |

ES: enrichment score; NES: Normalized enrichment score; NOM p-value: nominal p-value.

To investigate how CO reduces heme levels, whether CO decreased the transportation of heme into the cytosol was examined. It was found that 250 ppm CO reduced the mRNA expression of two key heme transporters, HRG1 and HCP1 (FIG. 4D). In contrast, no significant/consistent changes in a heme exporter, feline leukemia virus subgroup C receptor family member 1 (FLVCR1) were detected in these cell lines with or without 250 ppm CO treatment (FIG. 4D). Furthermore, heme oxygenases (HO-1, HO-2, and HO-3) cleave heme into 3 products: CO, ferrous ions (Fe2+), and biliverdin. Among the 3 heme oxygenases, only the expression, and hence activity, of HO-1 is inducible by biological, chemical, and physiological stress conditions caused by toxic concentrations of drugs. So, whether there was an increase of HO-1 expression by low-dose CO treatment that could contribute to heme degradation was examined. As shown in FIG. 4E, increase of HO-1 expression was not detected. Likewise, changes in protein levels of Bach 1 (a physiological repressor of HO-1 and a pro-metastatic transcription factor, whose degradation is reported to be mediated by heme in Nrf2-mutated lung cancer) was not detected (FIG. 4E). The results indicated that 250 ppm CO treatment of the two breast cancer cell lines do not increase the cellular production of endogenous CO through HO-1 upregulation. Taken together, the data suggested that the reduction of intracellular heme by low-dose CO is through downregulation of heme transporters, HRG1 and HCP1. Taken together, CO decreased heme importers to lower intracellular heme levels, which inhibited cancer cell migration.

The heme group confers functionality to multiple proteins, which can include oxygen carrying, oxygen reduction, electron transfer, and other processes. To determine whether the decrease of heme levels impacts levels of oxygen-utilizing hemoproteins and therefore reduces oxygen consumption, oxygen consumption rates (OCR) was measured. However, lower OCR after 16 hours in 250 ppm CO was not detected using Seahorse XF Cell Mito Stress Tests in MCF7 and MDA-MB-231 (data not shown).

Accordingly, compositions including low doses of carbon monoxide as disclosed herein are useful in methods for treating or preventing metastasis in a subject in need thereof.

### Example 6: Low-Dose CO Downregulates Expression of Cytochrome P450 Family 1 Subfamily B Member 1 (CYP1B1), Specificity Protein 1 (SP1), and Myc Target Genes

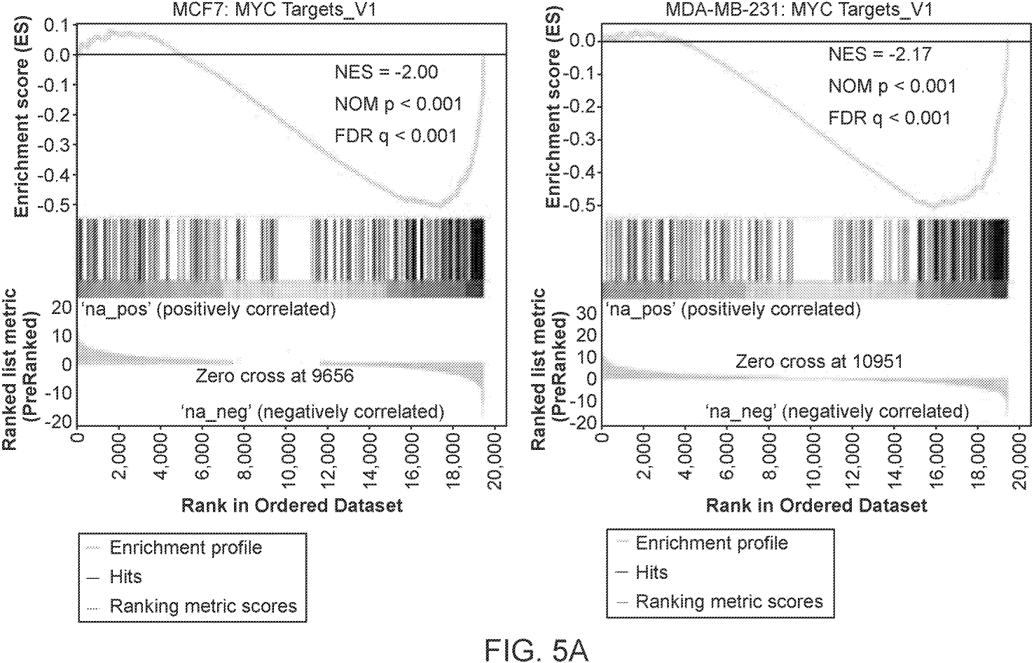

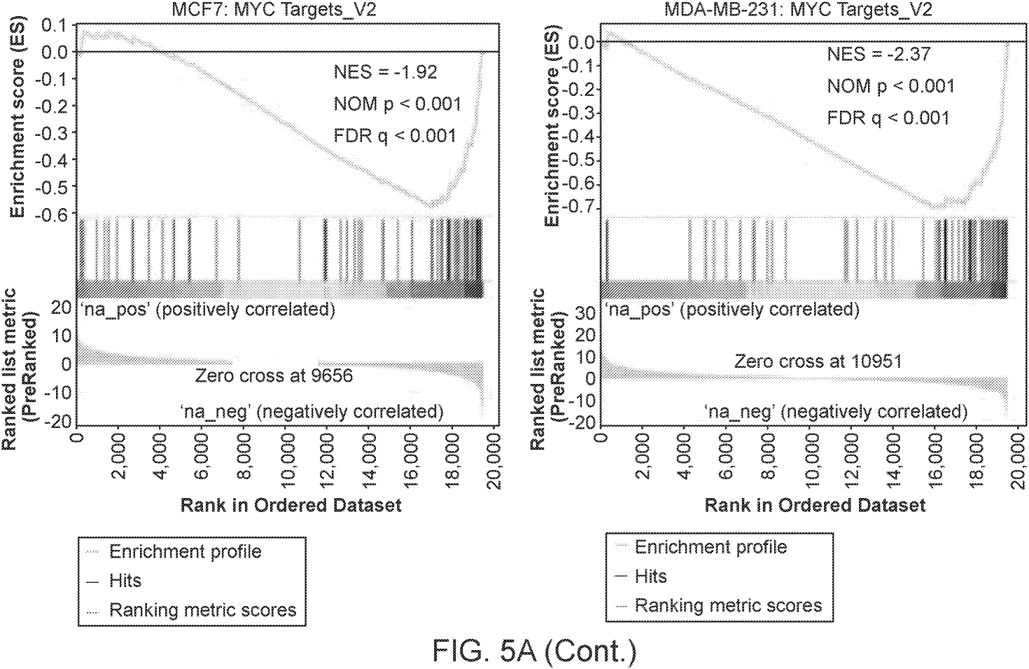

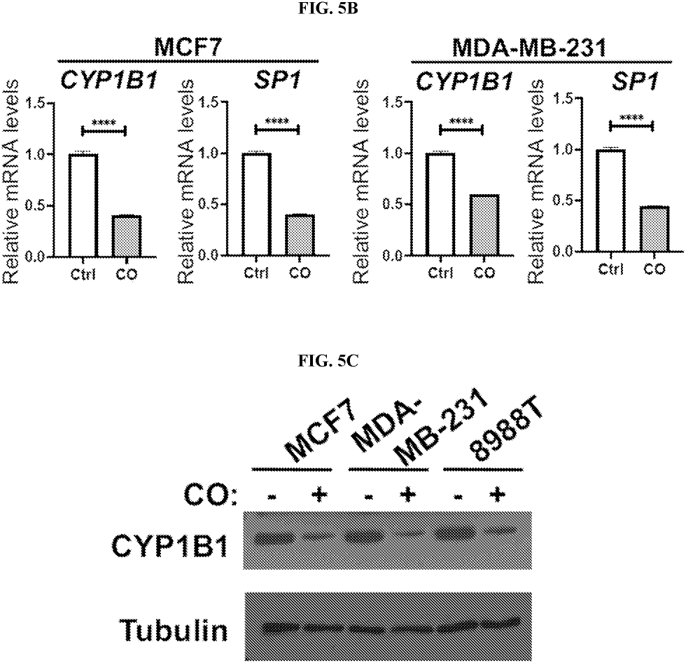

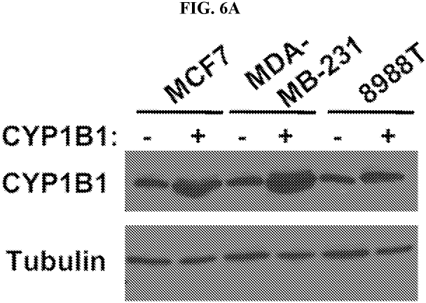

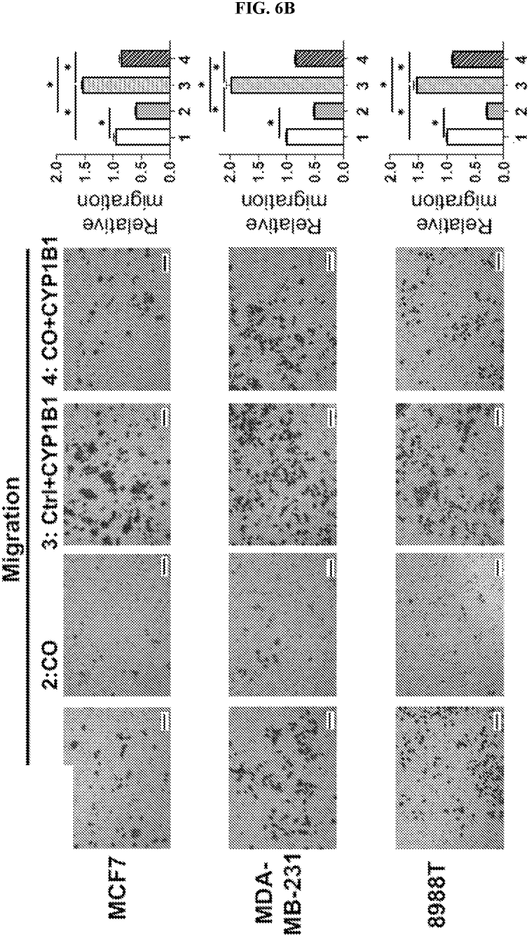

In addition to heme metabolism, the RNA-Seq/GSEA analysis revealed that low-dose CO downregulated HALLMARK_MYC_TARGETS_V1 and HALLMARK_MYC_TARGETS_V2, which consist of well-characterized genes whose transcriptions are directly regulated by the transcription factor c-Myc (Myc henceforth). Both gene sets were negatively correlated with low-dose CO, with a normalized enrichment score between -1.92 and -2.37 (FIG. 5A). The majority (~80%) of the members in both Myc target gene sets V1 and V2 were contributed to the enrichment score in MCF7 and MDA-MB-231 cells.

Heme is known to stimulate synthesis of many hemoproteins. Accordingly, hemoproteins whose expression was differentially regulated by low-dose CO in MCF7 and MDA-MB-231 cell lines were screened. CYP1B1, a heme-regulated protein, was identified as a top candidate in the list because of its following properties. First, CYP1B1 has been reported to induce the expression of SP1 transcription factor, and Sp1 interacts with Myc for synergistic transcriptional regulation for Myc target genes. Second, CYP1B1 is overexpressed in different types of cancer and its overexpression enhances breast cancer cell migration and invasion. Moreover, CYP1B1 overexpression has been associated with poor response to chemotherapy for TNBC patients and causes multiple drug resistance. Therefore, the effect of low-dose CO on the expression of CYP1B1 and SP1 was examined. CYP1B1 mRNA and protein levels, and SP1 mRNA levels were significantly reduced by 250 ppm CO in both MCF7 and MDA-MB-231 cells (FIGS. 5B-5C). In addition, CYP1B1 protein levels were reduced in PDAC 8988T cells treated with 250 ppm CO (FIG. 5C).

Accordingly, compositions including low doses of carbon monoxide as disclosed herein are useful in methods for treating or preventing metastasis in a subject in need thereof.

### Example 7: Transient Expression of CYP1B1 Restores Breast Cancer Cell Migration Inhibited by CO

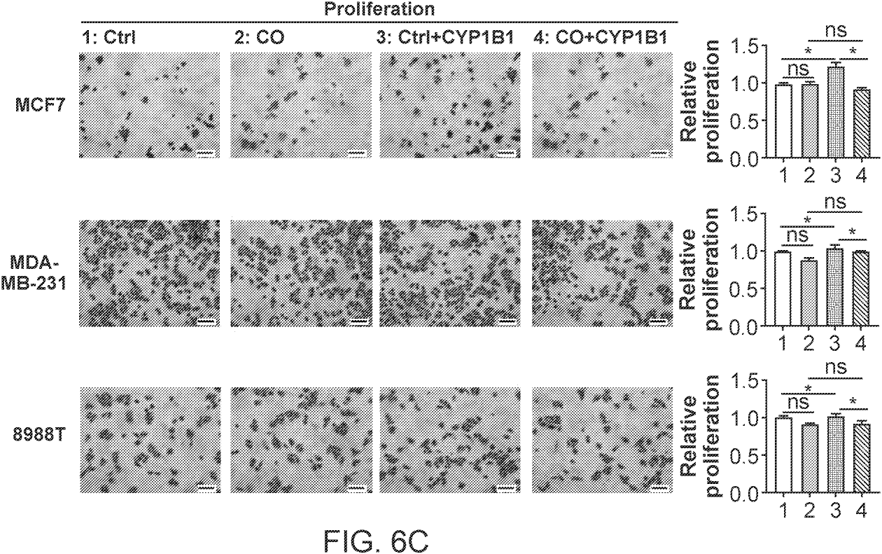

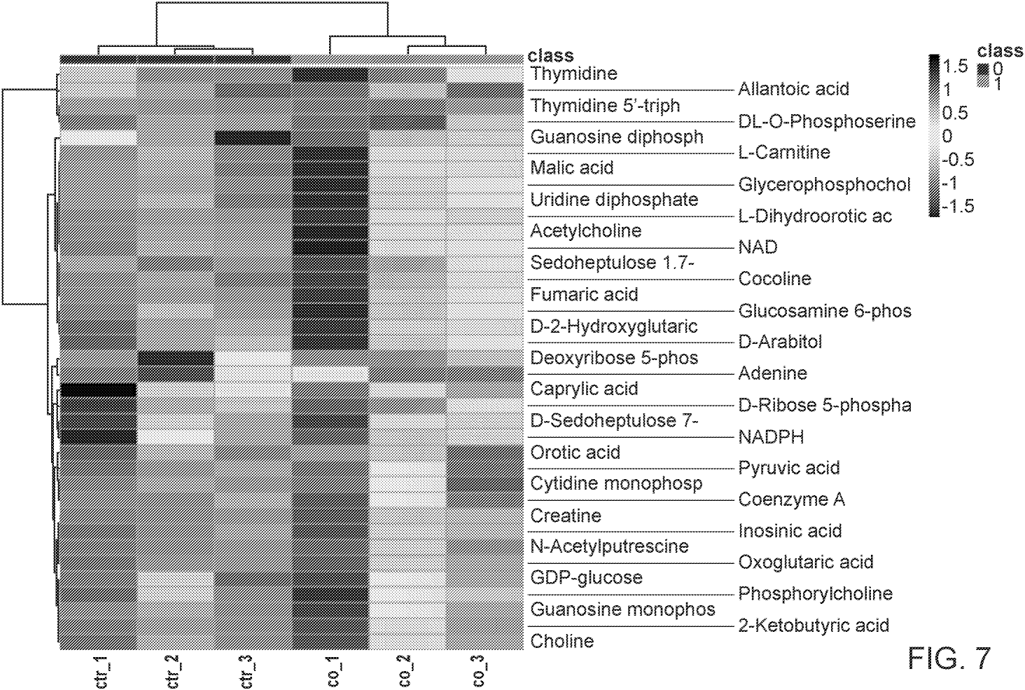

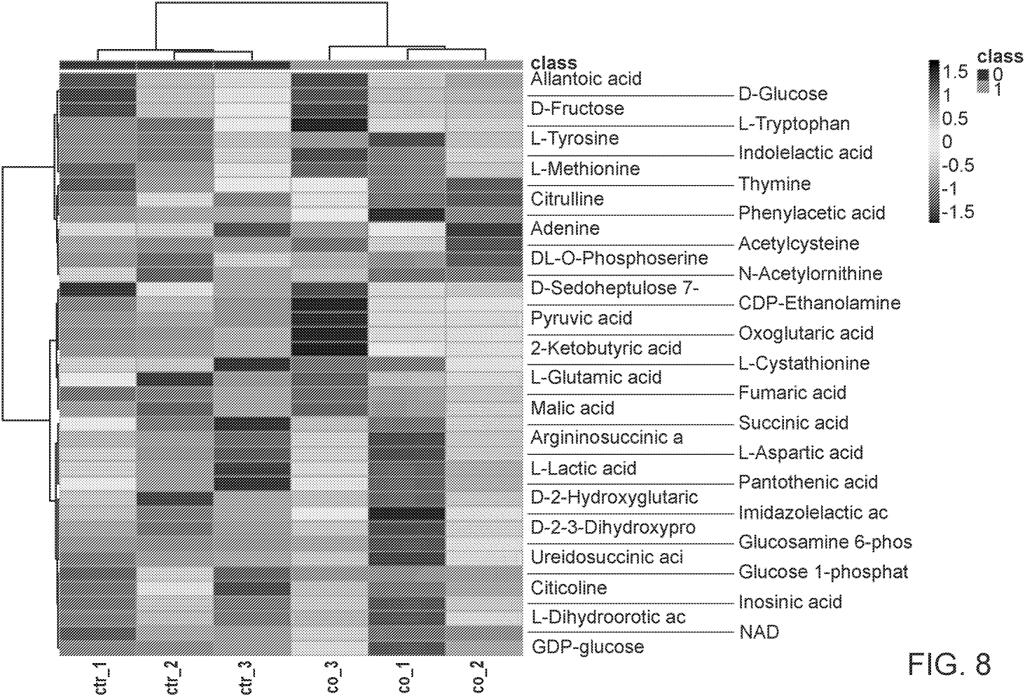

To test whether low-dose CO inhibits cell migration through downregulating CYP1B1, MCF7, MDA-MB-231, and 8988T cells were transiently transfected with a CYP1B1 expression vector. 48 hours after transfection, CYP1B1 proteins were overexpressed (FIG. 6A) and cells were seeded for transwell migration assays. Transient overexpression of CYP1B1 increased migration under the control condition and this restored migration was inhibited by low-dose CO (FIG. 6B). On the other hand, the effect of transient CYP1B1 overexpression on cell proliferation was not as profound (FIG. 6C).

### Example 8: Metabolomic Assay

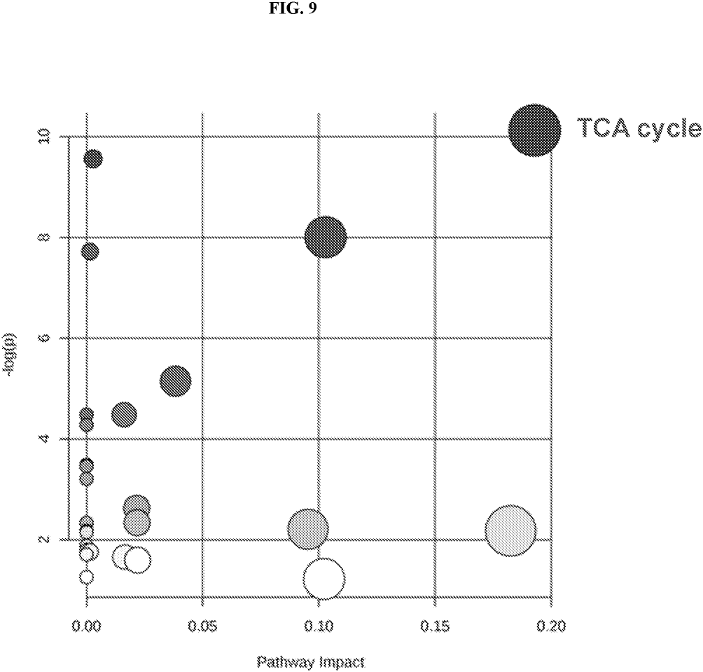

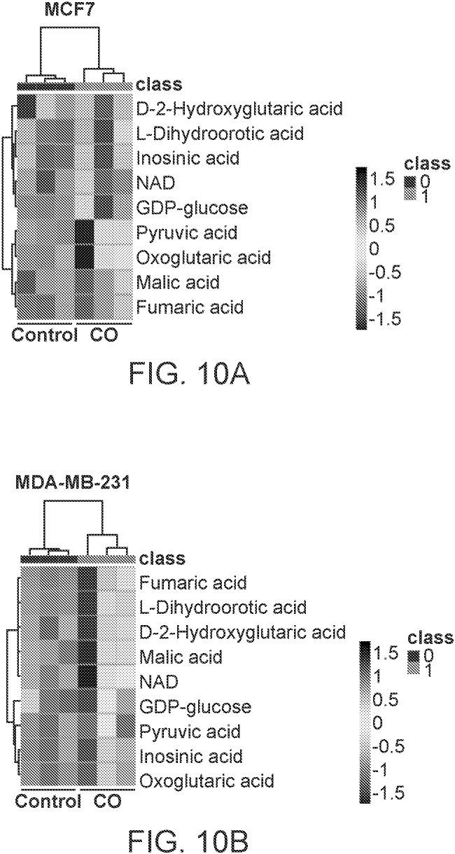

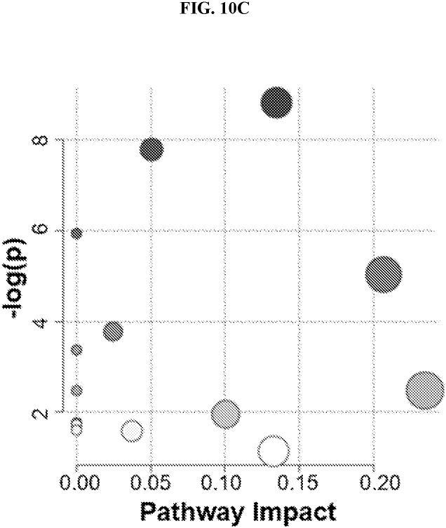

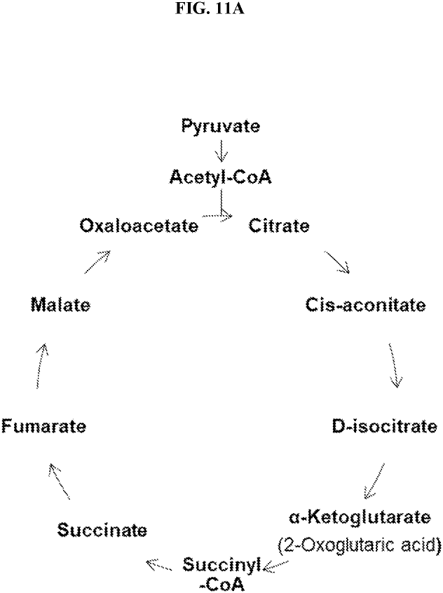

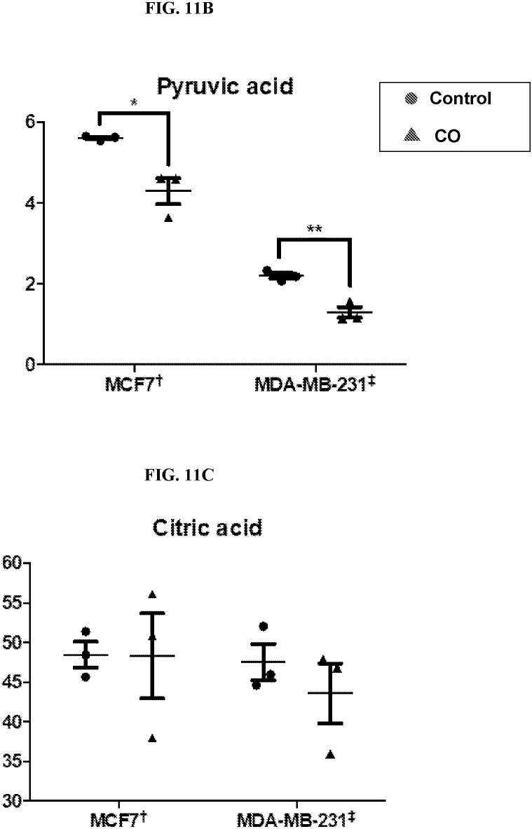

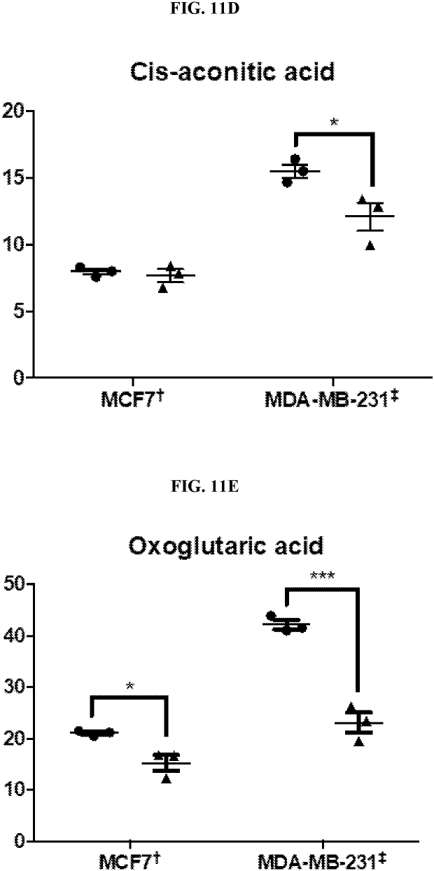

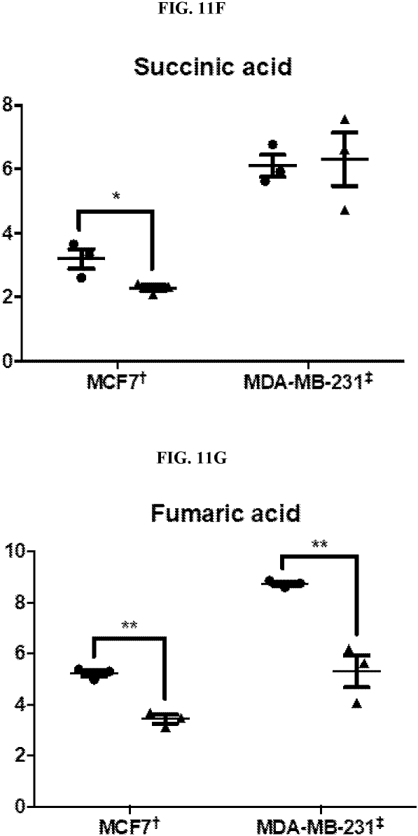

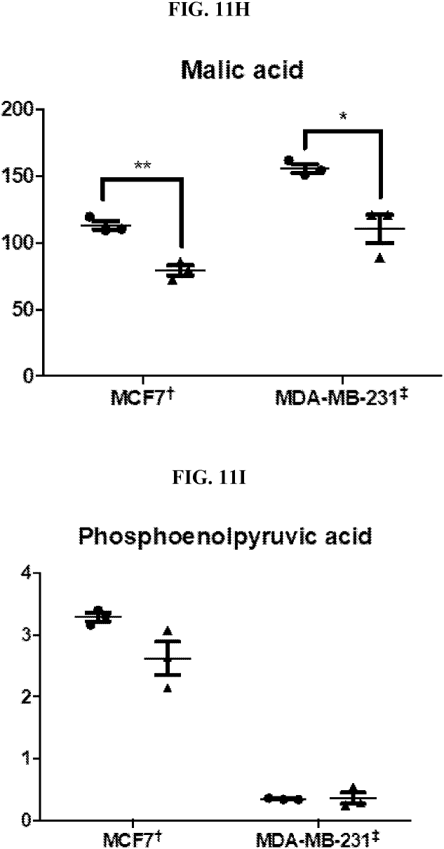

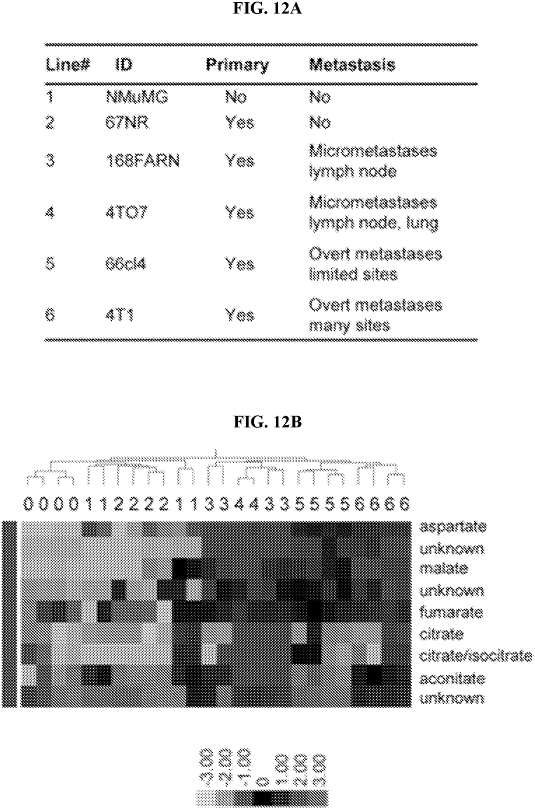

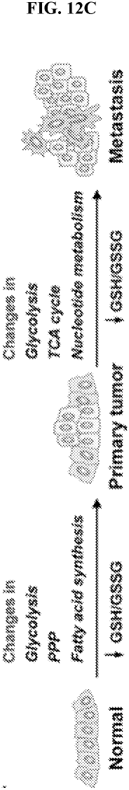

To investigate whether 250 ppm CO mimics hypoxia, the HiF1α protein levels were examined in CO treated MCF7/TGL and MDA-MB-231/TGL cells. Compared to the control cells grown in a standard tissue culture incubator, HiF1α protein levels were not changed after 250 ppm CO treatment for 16 hours (FIG. 4A), suggesting that this dose of CO did not induce hypoxia in tumor cells. To determine whether 250 ppm CO affects metabolism, polar metabolites were profiled using two breast cancer cell lines, MCF7/TGL and MDA-MB-231/TGL (FIGS. 7-9). Nine common metabolites were significantly downregulated by CO in both cell lines (FIG. 10A and FIG. 10B; Tables 1 and 2).

Pathway topology analysis using these nine compounds suggested that tricarboxylic acid (TCA) cycle was the most significantly downregulated pathway by 250 ppm CO (FIG. 10B and Table 5). All TCA intermediates detected in the polar metabolites profiling were then compared (FIG. 10C). Besides pyruvic acid, oxoglutaric acid, malic acid, and fumaric acid, which were significantly downregulated by CO in both cell lines, succinic acid was significantly downregulated by CO in MCF7/TGL and cis-aconitic acid was significantly downregulated by CO in MCF7/TGLMDA-MB-231/TGL cells (FIG. 10C, and FIGS. 11A-11I).

TABLE 3

Peak Height of the metabolite intensities determined by mass spectrometry from MCF7/TGL control cells and cells treated with CO

| | | | | | | | |
|---|---|---|---|---|---|---|---|
| HMDB | compound | control 1 | control 2 | control 3 | CO 1 | CO 2 | CO 3 |
| HMDB00641 | L-Glutamine | 1,771,223,000 | 1,470,929,000 | 2,040,454,000 | 2,010,884,000 | 2,067,120,000 | 2,186,859,000 |
| HMDB01565 | Phosphorylcholine | 923,709,100 | 839,445,100 | 1,030,611,000 | 826,434,500 | 950,902,100 | 1,062,045,000 |
| HMDB00162 | L-Proline | 493,155,700 | 453,239,900 | 534,918,700 | 456,504,200 | 470,321,900 | 561,265,500 |
| HMDB00064 | Creatine | 490,715,600 | 464,369,300 | 518,654,900 | 394,311,900 | 394,104,400 | 478,205,800 |
| HMDB00148 | L-Glutamic acid | 545,410,900 | 477,409,100 | 614,268,200 | 344,240,500 | 392,276,300 | 563,702,200 |
| HMDB00172 | L-Isoleucine | 334,641,300 | 299,436,700 | 376,419,500 | 339,480,000 | 333,493,200 | 481,854,600 |
| HMDB00687 | L-Leucine | 306,378,900 | 281,024,700 | 349,060,700 | 319,755,000 | 339,183,100 | 446,258,700 |
| HMDB00086 | Glycerophosphocholine | 390,112,000 | 380,402,400 | 459,501,100 | 299,915,900 | 348,438,900 | 459,259,100 |
| HMDB00177 | L-Histidine | 311,942,500 | 257,565,100 | 396,295,600 | 295,632,700 | 322,360,800 | 456,145,200 |
| HMDB00159 | L-Phenylalanine | 307,340,900 | 261,195,500 | 340,799,900 | 287,943,500 | 304,931,600 | 425,257,300 |
| HMDB00062 | L-Carnitine | 336,147,800 | 314,040,800 | 311,735,100 | 275,929,100 | 289,061,800 | 291,180,600 |
| HMDB00251 | Taurine | 213,144,700 | 172,842,200 | 224,507,000 | 213,761,300 | 213,696,600 | 188,154,600 |
| HMDB00043 | Betaine | 235,024,300 | 206,977,500 | 253,586,500 | 211,641,600 | 205,281,000 | 256,902,700 |
| HMDB00201 | L-Acetylcarnitine | 199,571,600 | 192,318,700 | 221,813,200 | 191,662,400 | 208,376,400 | 217,883,700 |
| HMDB00097 | Choline | 221,771,100 | 187,851,000 | 238,595,600 | 183,705,300 | 191,547,400 | 211,305,800 |
| HMDB00883 | L-Valine | 164,244,000 | 147,738,500 | 175,540,500 | 166,887,800 | 164,029,200 | 219,042,800 |
| HMDB00125 | Glutathione | 209,739,300 | 180,424,100 | 235,614,700 | 166,253,500 | 185,818,800 | 224,175,500 |
| HMDB00190 | L-Lactic acid | 269,679,700 | 222,772,500 | 337,663,600 | 135,248,600 | 168,348,200 | 288,458,500 |
| HMDB00744 | Malic acid | 236,769,000 | 197,474,300 | 246,846,100 | 118,667,000 | 141,604,200 | 190,900,500 |
| HMDB00696 | L-Methionine | 110,127,900 | 90,181,280 | 136,524,200 | 110,122,300 | 114,069,500 | 202,567,100 |
| HMDB00812 | N-Acetyl-L-aspartic acid | 143,511,900 | 133,502,700 | 151,285,200 | 107,096,600 | 121,628,700 | 146,462,300 |
| HMDB00167 | L-Threonine | 97,822,550 | 89,436,340 | 114,803,100 | 103,403,600 | 114,367,100 | 144,573,600 |
| HMDB00123 | Glycine | 63,492,120 | 52,307,860 | 65,584,360 | 97,069,490 | 60,912,580 | 81,769,040 |
| HMDB00824 | Propionylcarnitine | 109,740,900 | 92,085,490 | 123,376,800 | 88,247,270 | 88,089,220 | 114,107,300 |
| HMDB00094 | Citric acid | 103,874,900 | 84,892,510 | 102,780,000 | 83,266,830 | 83,863,100 | 100,258,700 |
| HMDB00895 | Acetylcholine | 85,738,300 | 67,042,970 | 78,856,880 | 79,843,420 | 75,982,720 | 87,382,500 |
| HMDB00943 | Threonic acid | 102,441,500 | 87,726,810 | 119,148,300 | 76,882,710 | 94,196,330 | 129,619,900 |
| HMDB00267 | Pyroglutamic acid | 82,273,380 | 89,079,720 | 124,855,200 | 70,921,790 | 82,143,660 | 125,886,000 |
| HMDB00517 | L-Arginine | 78,661,590 | 64,281,700 | 92,582,780 | 64,328,130 | 65,745,300 | 95,595,630 |
| HMDB00158 | L-Tyrosine | 73,471,940 | 56,196,120 | 79,025,800 | 58,933,980 | 62,662,290 | 101,701,400 |
| HMDB00210 | Pantothenic acid | 77,626,140 | 61,974,660 | 87,484,100 | 49,604,040 | 57,015,540 | 93,443,310 |
| HMDB00168 | L-Asparagine | 49,110,610 | 43,305,880 | 57,215,870 | 42,196,450 | 47,225,620 | 55,751,170 |
| HMDB00182 | L-Lysine Uridine diphosphate-N- | 59,257,100 | 47,797,510 | 69,556,820 | 41,138,540 | 40,717,620 | 74,426,720 |
| HMDB00290 | acetylglucosamine | 51,389,100 | 40,479,790 | 54,817,580 | 38,715,250 | 42,157,110 | 45,820,290 |
| HMDB00038 | Dihydrobiopterin | 39,014,920 | 37,740,640 | 48,284,760 | 37,904,750 | 45,778,470 | 54,551,140 |
| HMDB00929 | L-Tryptophan | 34,784,890 | 25,829,510 | 43,094,220 | 30,996,070 | 34,786,840 | 68,643,900 |
| HMDB00208 | Oxoglutaric acid | 46,142,950 | 35,028,210 | 46,046,030 | 24,674,800 | 27,743,170 | 32,411,680 |
| HMDB00122 | D-Glucose | 21,780,720 | 20,809,050 | 30,483,410 | 24,253,400 | 27,009,740 | 49,287,950 |
| HMDB00161 | L-Alanine | 32,110,330 | 28,877,910 | 34,362,530 | 24,164,680 | 26,271,210 | 37,606,940 |
| HMDB00187 | L-Serine | 20,618,210 | 16,561,370 | 17,726,500 | 22,682,210 | 24,881,370 | 27,375,240 |
| HMDB00562 | Creatinine | 22,137,860 | 19,143,220 | 28,968,350 | 20,992,210 | 21,746,470 | 37,545,350 |
| HMDB00235 | Thiamine | 17,602,240 | 15,812,710 | 20,360,820 | 13,320,550 | 15,145,370 | 21,979,980 |
| HMDB00099 | L-Cystathionine | 22,292,300 | 17,353,490 | 26,149,060 | 13,082,840 | 16,491,010 | 22,978,520 |
| HMDB00191 | L-Aspartic acid | 31,807,710 | 27,459,160 | 40,721,320 | 12,945,220 | 18,829,880 | 30,552,150 |
| HMDB00072 | cis-Aconitic acid | 16,294,420 | 13,244,410 | 18,695,220 | 11,654,680 | 13,826,410 | 17,813,720 |
| HMDB01864 | 2-Ketohexanoic acid | 18,769,390 | 14,916,610 | 24,470,640 | 10,109,740 | 13,290,500 | 25,544,760 |
| HMDB01185 | S-Adenosylmethionine | 17,442,410 | 11,656,970 | 14,135,130 | 9,310,209 | 12,590,700 | 17,427,170 |
| HMDB00904 | Citrulline | 9,353,630 | 8,094,927 | 9,883,847 | 9,044,262 | 10,234,370 | 14,264,050 |
| HMDB00565 | Galactonic acid | 14,145,730 | 9,980,662 | 15,776,030 | 8,090,935 | 11,406,370 | 14,258,950 |
| HMDB00262 | Thymine | 7,334,061 | 6,275,720 | 9,935,012 | 7,867,191 | 9,149,847 | 12,133,270 |
| HMDB00243 | Pyruvic acid | 12,079,550 | 9,175,009 | 12,737,430 | 6,830,386 | 7,616,124 | 9,628,515 |
| HMDB00092 | Dimethylglycine | 11,318,370 | 11,047,460 | 12,025,680 | 6,279,555 | 7,567,884 | 7,137,536 |
| HMDB00625 | Gluconic acid | 11,618,800 | 10,356,110 | 15,216,270 | 6,277,480 | 8,930,573 | 14,396,540 |
| HMDB00902 | NAD | 14,669,140 | 10,213,260 | 14,110,560 | 6,156,942 | 7,187,370 | 12,756,100 |
| HMDB00355 | 3-Hydroxymethylglutaric acid | 2,346,122 | 1,895,241 | 2,940,315 | 5,766,106 | 4,010,368 | 3,996,699 |
| HMDB00510 | Aminoadipic acid | 5,768,617 | 4,536,375 | 7,007,452 | 5,713,433 | 5,346,300 | 7,560,774 |
| HMDB00134 | Fumaric acid | 11,529,370 | 8,746,969 | 11,200,630 | 5,149,166 | 6,086,688 | 8,204,115 |
| HMDB00965 | Hypotaurine N6,N6,N6-Trimethyl-L- | 5,365,096 | 4,353,678 | 6,153,072 | 4,796,295 | 5,519,371 | 5,943,089 |
| HMDB01325 | lysine | 5,606,923 | 4,774,520 | 6,981,410 | 4,594,085 | 5,317,512 | 7,697,279 |
| HMDB00005 | 2-Ketobutyric acid | 7,102,009 | 5,409,002 | 7,235,881 | 4,031,318 | 4,391,029 | 5,247,754 |
| HMDB00482 | Caprylic acid Uridine diphosphate | 3,386,042 | 1,795,709 | 1,846,776 | 3,978,947 | 2,718,335 | 2,586,172 |
| HMDB00286 | glucose | 7,493,418 | 3,240,287 | 6,286,122 | 3,510,195 | 3,063,631 | 4,862,560 |
| HMDB00214 | Ornithine Phosphoenolpyruvic | 4,285,268 | 3,414,635 | 4,289,248 | 3,323,027 | 3,678,113 | 4,669,739 |
| HMDB00263 | acid | 7,293,509 | 5,214,385 | 7,437,299 | 3,181,575 | 5,081,852 | 6,959,276 |
| HMDB00292 | Xanthine | 3,460,680 | 2,994,270 | 4,991,982 | 3,106,304 | 2,715,740 | 5,495,149 |
| HMDB00254 | Succinic acid | 5,589,127 | 5,470,045 | 8,212,610 | 3,099,054 | 3,989,967 | 6,160,312 |
| HMDB00766 | N-Acetyl-L-alanine | 3,765,866 | 3,265,227 | 4,018,532 | 3,050,982 | 3,282,590 | 5,343,814 |
| HMDB00033 | Carnosine Asymmetric | 2,242,672 | 1,884,233 | 2,133,205 | 2,611,797 | 2,457,728 | 2,635,558 |
| HMDB01539 | dimethylarginine D-2,3-Dihydroxypropanoic | 3,678,366 | 3,237,718 | 4,672,597 | 2,485,123 | 2,893,325 | 4,095,202 |
| HMDB31818 | acid | 4,986,779 | 4,012,743 | 5,287,546 | 2,390,333 | 3,141,171 | 4,932,187 |
| HMDB00289 | Uric acid | 2,571,347 | 2,061,890 | 3,634,430 | 2,364,329 | 2,512,157 | 4,422,182 |
| HMDB00660 | D-Fructose Methylimidazoleacetic | 2,234,813 | 2,004,556 | 2,912,983 | 2,161,466 | 2,426,370 | 4,390,968 |
| HMDB02820 | acid | 3,226,625 | 2,718,716 | 3,893,899 | 2,032,579 | 2,307,084 | 4,227,106 |
| HMDB00538 | Adenosine triphosphate | 2,950,496 | 1,188,375 | 1,938,541 | 1,853,382 | 1,283,500 | 2,002,755 |
| HMDB01401 | Glucose 6-phosphate | 3,154,593 | 2,411,959 | 3,247,078 | 1,799,795 | 2,339,699 | 3,140,759 |
| HMDB01406 | Niacinamide | 1,498,044 | 1,116,313 | 2,622,268 | 1,738,434 | 1,604,153 | 3,615,850 |
| HMDB11718 | 4-Hydroxybenzaldehyde | 1,963,473 | 1,654,208 | 2,255,978 | 1,638,500 | 1,711,975 | 2,780,422 |
| HMDB11745 | N-Acetyl-L-methionine | 1,924,654 | 3,501,060 | 2,262,447 | 1,517,454 | 1,648,819 | 2,302,821 |
| HMDB00211 | Myoinositol | 2,272,971 | 2,078,419 | 2,959,535 | 1,493,285 | 1,572,114 | 2,893,583 |
| HMDB00462 | Allantoin D-Glyceraldehyde 3- | 2,144,143 | 1,678,035 | 2,250,925 | 1,483,552 | 1,936,773 | 3,316,178 |
| HMDB01112 | phosphate | 1,590,290 | 1,276,881 | 1,649,088 | 1,475,183 | 1,160,887 | 1,058,732 |
| HMDB00821 | Phenylacetylglycine | 1,211,287 | 1,071,523 | 1,674,497 | 1,455,001 | 1,300,695 | 2,222,377 |
| HMDB03337 | Oxidized glutathione | 1,810,877 | 1,344,020 | 2,050,688 | 1,351,310 | 1,735,245 | 4,066,758 |
| HMDB00044 | Ascorbic acid | 4,462,647 | 2,835,430 | 5,833,235 | 1,179,857 | 2,029,092 | 5,154,497 |
| HMDB02005 | Methionine sulfoxide | 1,286,593 | 1,244,997 | 1,822,228 | 1,119,307 | 1,490,306 | 2,521,401 |
| HMDB01413 | Citicoline | 2,554,237 | 1,652,364 | 2,848,083 | 1,054,917 | 1,269,775 | 2,126,892 |
| HMDB00285 | Uridine triphosphate | 1,647,539 | 686,091 | 1,135,856 | 972,321 | 740,370 | 1,159,635 |
| HMDB00828 | Ureidosuccinic acid | 5,533,934 | 3,904,229 | 5,201,183 | 967,259 | 2,115,336 | 2,977,458 |
| HMDB01341 | ADP | 1,074,820 | 652,124 | 953,749 | 902,793 | 705,422 | 1,082,377 |
| HMDB00807 | 3-Phosphoglyceric acid Uridine diphosphate | 2,012,371 | 1,068,096 | 2,210,814 | 830,372 | 1,313,918 | 2,225,910 |
| HMDB00935 | glucuronic acid | 1,195,086 | 548,383 | 901,285 | 726,138 | 548,546 | 770,082 |
| HMDB00124 | Fructose 6-phosphate | 1,082,608 | 815,636 | 1,216,530 | 689,466 | 869,013 | 1,084,547 |
| HMDB00960 | dGDP | 993,826 | 504,561 | 686,837 | 686,954 | 539,654 | 784,042 |
| HMDB01624 | 2-Hydroxycaproic acid | 967,571 | 645,854 | 874,213 | 663,141 | 926,482 | 905,559 |
| HMDB00300 | Uracil Adenosine | 702,546 | 601,270 | 796,368 | 621,176 | 684,209 | 956,261 |
| HMDB00045 | monophosphate | 774,386 | 696,168 | 802,211 | 595,508 | 473,948 | 497,428 |
| HMDB01138 | N-Acetylglutamic acid 4-Acetamidobutanoic | 1,361,525 | 995,688 | 1,568,929 | 567,078 | 897,146 | 1,371,040 |
| HMDB03681 | acid | 603,191 | 553,360 | 776,328 | 550,530 | 718,720 | 878,912 |
| HMDB01548 | D-Ribose 5-phosphate N-Acetyl-glucosamine | 829,467 | 740,237 | 762,826 | 523,976 | 578,358 | 695,562 |
| HMDB01367 | 1-phosphate D-2-Hydroxyglutaric | 561,406 | 421,587 | 620,347 | 464,872 | 467,412 | 595,300 |
| HMDB00606 | acid | 865,256 | 753,377 | 942,554 | 460,608 | 570,887 | 905,587 |
| HMDB01066 | S-Lactoylglutathione | 493,987 | 382,740 | 603,024 | 397,298 | 293,636 | 484,692 |
| HMDB00779 | Phenyllactic acid | 346,207 | 271,224 | 372,282 | 375,745 | 377,557 | 505,569 |
| HMDB03357 | N-Acetylornithine | 462,986 | 330,625 | 472,464 | 364,451 | 403,207 | 622,448 |
| HMDB00301 | Urocanicacid | 324,044 | 367,712 | 141,820 | 330,874 | 577,556 | 198,149 |
| HMDB00034 | Adenine | 432,446 | 330,792 | 401,561 | 319,396 | 405,129 | 608,706 |
| HMDB00705 | Hexanoylcarnirine | 488,970 | 362,128 | 540,437 | 309,788 | 384,626 | 554,943 |
| HMDB01890 | Acetylcysteine | 310,746 | 225,567 | 320,265 | 309,165 | 445,217 | 662,532 |
| HMDB00295 | Uridine 5′-diphosphate | 488,182 | 191,209 | 373,772 | 297,075 | 234,752 | 314,015 |
| HMDB06028 | N-Acetylasparagine | 244,172 | 246,132 | 211,297 | 279,146 | 204,547 | 192,934 |
| HMDB01173 | 5′-Methylthioadenosine | 842,189 | 661,790 | 1,099,702 | 269,714 | 419,042 | 1,274,477 |
| HMDB01227 | 5-Thymidylic acid | 97,686 | 104,783 | 139,862 | 266,456 | 133,281 | 141,715 |
| HMDB00186 | Alpha-Lactose | 281,362 | 308,145 | 305,333 | 261,525 | 290,474 | 303,327 |
| HMDB01721 | DL-O-Phosphoserine | 261,481 | 189,989 | 302,802 | 256,439 | 305,155 | 440,561 |
| HMDB00568 | D-Arabitol | 261,667 | 251,812 | 319,602 | 250,315 | 263,626 | 418,856 |
| HMDB00082 | Cytidine triphosphate | 334,768 | 151,645 | 197,918 | 248,084 | 155,802 | 221,285 |
| HMDB00126 | Glycerol3-phosphate | 486,286 | 409,144 | 556,296 | 242,288 | 358,077 | 520,107 |
| HMDB06029 | N-Acetylglutamine | 279,292 | 234,354 | 331,629 | 233,588 | 221,551 | 457,769 |
| HMDB01564 | CDP-Ethanolamine | 419,589 | 300,542 | 435,991 | 229,328 | 262,826 | 313,255 |
| HMDB00157 | Hypoxanthine | 59,055 | 51,081 | 47,541 | 207,108 | 78,064 | 86,812 |
| HMDB01586 | Glucose 1-phosphate | 454,213 | 297,361 | 474,259 | 194,892 | 220,042 | 348,617 |
| HMDB00127 | D-Glucuronic acid | 221,976 | 205,556 | 291,847 | 193,639 | 229,627 | 245,391 |
| HMDB00684 | L-Kynurenine | 286,050 | 177,819 | 324,183 | 186,534 | 218,684 | 386,445 |
| HMDB00052 | Argininosuccinic acid | 570,420 | 464,496 | 707,865 | 180,070 | 286,271 | 472,100 |
| HMDB00671 | Indolelactic acid | 158,936 | 118,444 | 189,673 | 168,415 | 166,030 | 323,966 |
| HMDB00630 | Cytosine D-Sedoheptulose 7- | 165,131 | 131,570 | 220,325 | 153,235 | 158,066 | 214,404 |
| HMDB01068 | phosphate | 269,170 | 181,629 | 260,536 | 151,546 | 165,071 | 234,010 |
| HMDB02320 | Imidazolelactic acid | 362,911 | 311,887 | 393,464 | 128,890 | 215,532 | 355,864 |
| HMDB00206 | N6-Acetyl-L-lysine | 188,957 | 182,776 | 196,885 | 126,221 | 143,548 | 210,190 |
| HMDB02994 | Erythritol | 151,450 | 131,971 | 194,055 | 124,085 | 149,580 | 273,411 |
| HMDB00089 | Cytidine | 68,833 | 52,277 | 92,894 | 104,165 | 69,105 | 173,745 |
| HMDB00095 | Cytidine monophosphate | 164,566 | 113,718 | 147,020 | 93,671 | 115,860 | 141,423 |
| HMDB01273 | Guanosine triphosphate | 158,867 | 62,365 | 122,109 | 84,003 | 60,453 | 112,154 |
| HMDB01487 | NADH Guanosine | 91,975 | 52,695 | 68,007 | 81,979 | 73,214 | 61,204 |
| HMDB01397 | monophosphate | 104,958 | 63,750 | 98,638 | 73,405 | 58,750 | 80,249 |
| HMDB00715 | Kynurenic acid S- | 60,504 | 49,674 | 59,560 | 57,830 | 46,636 | 67,981 |
| HMDB00939 | Adenosylhomocysteine Thymidine 5′- | 238,341 | 167,981 | 396,613 | 56,652 | 145,088 | 413,677 |
| HMDB01342 | triphosphate | 45,446 | 20,989 | 44,842 | 54,889 | 34,363 | 35,046 |
| HMDB03349 | L-Dihydroorotic acid | 164,653 | 112,848 | 175,246 | 52,713 | 82,695 | 121,047 |
| HMDB06557 | ADP-glucose | 91,989 | 47,867 | 71,590 | 51,484 | 47,964 | 81,136 |
| HMDB00296 | Uridine | 45,579 | 36,147 | 47,587 | 51,402 | 29,724 | 61,581 |
| HMDB01546 | CDP | 54,846 | 38,904 | 40,600 | 49,666 | 36,830 | 44,004 |
| HMDB01209 | Allantoic acid Glucosamine 6- | 41,618 | 38,718 | 56,469 | 44,843 | 53,026 | 96,308 |
| HMDB01254 | phosphate | 101,778 | 78,916 | 106,086 | 44,392 | 62,516 | 90,486 |
| HMDB00714 | Hippuric acid | 35,005 | 28,079 | 43,120 | 41,839 | 33,037 | 50,895 |
| HMDB00217 | NADP | 57,912 | 33,883 | 53,826 | 35,994 | 44,669 | 56,121 |
| HMDB01201 | Guanosine diphosphate Uridine 5′- | 54,616 | 27,531 | 45,707 | 35,607 | 34,452 | 43,718 |
| HMDB00288 | monophosphate | 55,627 | 35,516 | 54,955 | 35,559 | 34,351 | 45,597 |
| HMDB00682 | Indoxyl sulfate | 27,520 | 19,928 | 30,717 | 31,554 | 19,025 | 38,019 |
| HMDB03351 | GDP-glucose | 74,355 | 56,641 | 75,845 | 30,304 | 39,328 | 68,617 |
| HMDB00175 | Inosinic acid | 47,607 | 33,853 | 49,601 | 22,764 | 28,902 | 46,283 |
| HMDB01031 | Deoxyribose 5-phosphate | 40,053 | 40,176 | 61,897 | 19,019 | 34,976 | 30,540 |
| HMDB00273 | Thymidine | 9,703 | 4,443 | 10,144 | 18,849 | 10,836 | 11,271 |
| HMDB00050 | Adenosine 3-hydroxyhexadecanoic | 21,223 | 12,760 | 20,573 | 14,622 | 14,037 | 23,296 |
| HMDB61658 | acid | 13,102 | 12,036 | 11,635 | 11,142 | 13,344 | 11,314 |
| HMDB00244 | Riboflavin | 8,946 | 23,373 | 28,502 | 8,515 | - | 36,944 |
| HMDB00012 | Deoxyuridine | 0,104 | 7,281 | 3,701 | 4,052 | 3,614 | 4,205 |
| HMDB00128 | Guanidoacetic acid | - | - | - | - | - | - |
| HMDB01316 | 6-Phosphogluconic acid | - | - | - | - | - | - |
| HMDB01520 | Flavin Mononucleotide | - | - | - | - | - | - |
| HMDB01015 | N-Formyl-L-methionine | - | - | - | - | - | - |
| HMDB03335 | IDP | - | - | - | - | - | - |
| HMDB00710 | 4-Hydroxybutyric acid | - | - | - | - | - | - |
| HMDB00133 | Guanosine | - | - | - | - | - | - |
| HMDB00150 | Gluconolactone | - | - | - | - | - | - |
| HMDB00653 | Cholesterol sulfate | - | - | - | - | - | - |
| HMDB00764 | Hydrocinnamic acidBeta-D- | - | - | - | - | - | - |
| HMDB10314 | Glucopyranuronic acid | - | - | - | - | - | - |
| HMDB06116 | 3-Hydroxyhippuric acid | - | - | - | - | - | - |
| HMDB03320 | Indole-3-carboxylic acid | - | - | - | - | - | - |
| HMDB00220 | Palmitic acid | - | - | - | - | - | - |
| HMDB00221 | NADPH | - | - | - | - | - | - |
| HMDB00618 | D-Ribulose 5-phosphate | - | - | - | - | - | - |
| HMDB13713 | N-acetyltryptophan | - | - | - | - | - | - |
| HMDB00226 | Orotic acid | 301,686 | 190,857 | - | - | 107,743 | 166,715 |
| HMDB02302 | 3-Indolepropionic acid | - | - | - | - | - | - |
| HMDB00881 | Xanthurenic acid | - | - | - | - | - | - |
| HMDB01248 | FAD 2-Oxo-4- | - | - | - | - | - | - |
| HMDB01553 | methylthiobutanoic acid | - | - | - | - | - | - |
| HMDB00017 | 4-Pyridoxic acidTaurochenodesoxycholic | - | - | - | - | - | - |
| HMDB00951 | acid | - | - | - | - | - | - |
| HMDB00132 | Guanine | - | - | - | - | - | - |
| HMDB00283 | D-Ribose Adenosine | - | - | - | - | - | - |
| HMDB01003 | phosphosulfate | 19,617 | 9,973 | 22,699 | - | 14,981 | 16,338 |
| HMDB01206 | Acetyl-CoA Fructose 1,6- | - | - | - | - | - | - |
| HMDB01058 | bisphosphate | - | - | - | - | - | - |
| HMDB01202 | dCMP | - | - | - | - | - | - |
| HMDB01366 | Purine | - | - | - | - | - | - |
| HMDB01245 | dCDP | - | - | - | - | - | - |
| HMDB01517 | AICAR | - | - | - | - | - | - |
| HMDB00699 | 1-Methylnicotinamide | - | - | - | - | - | - |
| HMDB00998 | dCTP | - | - | - | - | - | - |
| HMDB06372 | L-Glyceric acid | - | - | - | - | - | - |
| HMDB00742 | Homocysteine | - | - | - | - | - | - |
| HMDB00071 | Deoxyinosine | - | - | - | - | - | - |
| HMDB00619 | Cholic acid | - | - | - | - | - | - |
| HMDB00310 | Methylacetoacetic acid | - | - | - | - | - | - |
| HMDB00101 | Deoxyadenosine | - | - | - | - | - | - |
| HMDB00229 | Nicotinamide ribotide Deoxyadenosine | - | - | - | - | - | - |
| HMDB00905 | monophosphate | - | - | - | - | - | - |
| HMDB01107 | 7-Methylguanosine | - | - | - | - | - | - |
| HMDB01554 | Xanthylic acid | - | - | - | - | - | - |
| HMDB01863 | 2-Hydroxyvaleric acidD-Erythrose 4- | - | - | - | - | - | - |
| HMDB01321 | phosphate | - | - | - | - | - | - |
| HMDB00574 | L-Cysteine 6-Phosphonoglucono-D- | - | - | - | - | - | - |
| HMDB01127 | lactone | - | - | - | - | - | - |
| HMDB01043 | Arachidonic acid | - | - | - | - | - | - |
| HMDB01514 | Glucosamine | - | - | - | - | - | - |
| HMDB00222 | L-Palmitoylcarnitine | - | - | - | - | - | - |
| HMDB01423 | Coenzyme AThiamine | - | - | - | - | - | - |
| HMDB02666 | monophosphateDeoxyadenosine | - | - | - | - | - | - |
| HMDB01532 | triphosphate | - | - | - | - | - | - |
| HMDB00195 | Inosine | - | - | - | - | - | - |
| HMDB03331 | 1-Methyladenosine | - | - | - | - | - | - |
| HMDB00738 | Indole3-Methylthiopropionic | - | - | - | - | - | - |
| HMDB01527 | acid | - | - | - | - | - | - |
| HMDB01022 | Succinyl-CoA | - | - | - | - | - | - |
| HMDB02339 | 5-Methoxytryptophan N-Acetylaspartylglutamic | - | - | - | - | - | - |
| HMDB01067 | acidSedoheptulose 1,7- | - | - | - | - | - | - |
| HMDB60274 | bisphosphate | - | - | - | - | - | - |
| HMDB00239 | Pyridoxine | - | - | - | - | - | - |
| HMDB00223 | Oxalacetic acidN-Acetyl-D- | - | - | - | - | - | - |
| HMDB00215 | glucosamine | - | - | - | - | - | - |
| HMDB00192 | L-Cystine | - | - | - | - | - | - |

TABLE 4

Peak Height of the metabolite intensities determined by mass spectrometry from MDA-MB-231/TGL control cells and cells treated with CO

| | | | | | | | |
|---|---|---|---|---|---|---|---|
| HMDB | compound | control 1 | control 2 | control 3 | CO 1 | CO 2 | CO 3 |
| HMDB00641 | L-Glutamine | 1,519,698,000 | 1,317,401,000 | 1,408,149,000 | 1,601,450,000 | 1,636,785,000 | 1,523,599,000 |
| HMDB01565 | Phosphorylcholine | 1,002,988,000 | 973,819,600 | 980,939,600 | 926,340,400 | 954,637,100 | 1,004,940,000 |
| HMDB00148 | L-Glutamic acid | 891,714,600 | 828,287,300 | 850,068,400 | 655,681,000 | 727,466,400 | 916,546,500 |
| HMDB00162 | L-Proline | 651,850,100 | 636,705,400 | 619,884,400 | 612,301,800 | 622,349,000 | 718,183,500 |
| HMDB00086 | Glycerophosphocholine | 400,750,400 | 421,953,100 | 410,800,400 | 347,562,900 | 361,214,700 | 419,624,900 |
| HMDB00190 | L-Lactic acid | 327,049,700 | 330,170,900 | 350,176,600 | 289,904,000 | 340,883,500 | 383,011,600 |
| HMDB00687 | L-Leucine | 216,460,100 | 221,949,500 | 228,017,500 | 283,061,200 | 273,188,600 | 323,263,900 |
| HMDB00172 | L-Isoleucine | 235,544,900 | 228,716,700 | 233,097,000 | 271,615,500 | 275,153,400 | 327,851,400 |
| HMDB00251 | Taurine | 185,204,400 | 185,910,100 | 195,517,500 | 250,114,800 | 184,320,000 | 217,876,200 |
| HMDB00159 | L-Phenylalanine | 179,205,400 | 175,126,900 | 184,875,300 | 221,726,300 | 225,785,500 | 288,325,000 |
| HMDB00744 | Malic acid | 234,699,700 | 245,933,900 | 247,643,300 | 185,995,600 | 208,411,100 | 232,110,400 |
| HMDB00062 | L-Carnitine | 199,474,000 | 205,453,700 | 203,177,200 | 180,708,600 | 186,528,100 | 206,022,400 |
| HMDB00167 | L-Threonine | 105,409,800 | 109,284,900 | 107,023,400 | 137,793,200 | 134,164,900 | 175,180,900 |
| HMDB00043 | Betaine | 116,160,800 | 118,443,100 | 116,730,000 | 126,207,200 | 122,297,900 | 134,080,700 |
| HMDB00064 | Creatine | 134,949,300 | 143,877,000 | 133,554,600 | 123,445,200 | 119,145,800 | 129,072,700 |
| HMDB00177 | L-Histidine | 79,941,010 | 82,378,970 | 89,573,570 | 120,776,800 | 113,578,300 | 158,867,000 |
| HMDB00883 | L-Valine | 99,138,230 | 107,665,900 | 99,229,900 | 112,753,800 | 120,589,500 | 133,489,100 |
| HMDB00097 | Choline | 122,368,300 | 122,052,500 | 119,463,800 | 108,427,800 | 109,880,900 | 111,858,100 |
| HMDB00943 | Threonic acid | 93,134,170 | 95,015,620 | 97,391,700 | 107,442,800 | 103,948,700 | 114,088,000 |
| HMDB00201 | L-Acetylcarnitine | 125,193,400 | 126,410,400 | 124,935,800 | 106,933,600 | 121,142,500 | 148,690,900 |
| HMDB00125 | Glutathione | 114,589,800 | 126,730,700 | 118,351,400 | 99,156,710 | 109,797,600 | 130,505,000 |
| HMDB00267 | Pyroglutamic acid | 94,076,690 | 80,631,580 | 93,876,140 | 95,285,270 | 98,786,160 | 116,343,300 |
| HMDB00696 | L-Methionine N-Acetyl-L-aspartic | 55,535,240 | 58,838,160 | 60,720,150 | 84,626,580 | 79,234,880 | 99,088,640 |
| HMDB00812 | acid | 96,694,600 | 96,267,140 | 99,801,960 | 82,644,790 | 91,504,070 | 102,958,000 |
| HMDB00094 | Citric acid | 67,851,860 | 74,695,950 | 79,569,970 | 74,805,210 | 82,630,710 | 89,689,600 |
| HMDB00210 | Pantothenic acid | 72,407,420 | 75,310,200 | 72,339,060 | 66,785,200 | 78,653,480 | 81,031,300 |
| HMDB00158 | L-Tyrosine | 53,373,840 | 51,816,570 | 51,161,000 | 62,958,740 | 63,148,990 | 80,943,860 |
| HMDB00187 | L-Serine | 32,882,550 | 33,020,870 | 34,023,590 | 47,600,670 | 48,117,350 | 49,703,860 |
| HMDB00123 | Glycine | 54,670,900 | 50,980,160 | 51,435,220 | 45,296,030 | 53,615,700 | 58,796,920 |
| HMDB00824 | Propionylcamitine | 47,603,880 | 56,133,070 | 9,386,740 | 42,262,630 | 48,738,950 | 53,591,860 |
| HMDB00208 | Oxoglutaric acid | 66,573,060 | 66,618,650 | 63,424,680 | 40,551,530 | 45,257,220 | 44,892,300 |
| HMDB00625 | Gluconic acid | 52,644,920 | 54,307,840 | 50,582,950 | 37,957,970 | 52,856,170 | 64,673,460 |
| HMDB00122 | D-Glucose | 27,042,700 | 22,661,390 | 22,557,500 | 36,851,430 | 39,288,740 | 42,369,140 |
| HMDB00895 | Acetylcholine | 42,252,070 | 43,371,880 | 41,406,850 | 34,254,070 | 37,448,230 | 42,218,910 |
| HMDB00517 | L-Arginine | 23,057,170 | 19,864,520 | 21,795,470 | 31,387,180 | 29,265,460 | 31,837,150 |
| HMDB00211 | Myoinositol | 42,448,430 | 39,496,060 | 38,654,730 | 29,719,830 | 34,248,940 | 40,470,300 |
| HMDB00929 | L-Tryptophan | 21,327,340 | 22,211,440 | 21,457,990 | 29,250,240 | 30,006,710 | 37,673,960 |
| HMDB00235 | Thiamine | 27,677,710 | 26,805,650 | 24,092,610 | 24,404,230 | 28,664,720 | 27,656,500 |
| HMDB00182 | L-Lysine | 21,805,100 | 20,986,590 | 21,131,650 | 24,306,150 | 24,075,250 | 28,401,380 |
| HMDB00562 | Creatinine | 18,115,390 | 20,789,150 | 21,135,020 | 23,236,710 | 23,501,360 | 25,483,820 |
| HMDB00168 | L-Asparagine | 15,698,750 | 15,296,350 | 16,632,330 | 21,992,250 | 21,453,690 | 27,389,900 |
| HMDB01864 | 2-Ketohexanoic acid | 26,463,340 | 22,014,150 | 22,106,630 | 21,644,820 | 23,261,950 | 24,254,29000072 |
| HMDB00072 | cis-Aconitic acid 3-Hydroxymethylglutaric | 23,591,410 | 23,858,160 | 25,060,430 | 20,740,980 | 23,097,840 | 24,649,890 |
| HMDB00355 | acid Uridine diphosphate-N- | 16,264,690 | 16,768,800 | 17,631,880 | 20,353,060 | 19,480,200 | 19,066,660 |
| HMDB00290 | acetylglucosamine | 26,389,750 | 27,375,900 | 29,770,390 | 20,151,160 | 26,809,930 | 32,502,900 |
| HMDB00050 | Adenosine | 351,588 | 603,187 | 868,240 | 18,973,870 | 624,270 | 424,559 |
| HMDB00902 | NAD | 25,525,460 | 25,403,870 | 24,488,200 | 17,332,970 | 21,051,080 | 23,992,650 |
| HMDB00904 | Citrulline | 14,517,660 | 13,478,890 | 14,402,990 | 15,240,380 | 14,266,830 | 19,605,850 |
| HMDB00092 | Dimethylglycine | 17,830,400 | 19,457,670 | 15,598,050 | 10,914,430 | 13,334,130 | 17,506,690 |
| HMDB00960 | dGDP Fructose 1,6- | 8,852,598 | 10,482,160 | 11,325,820 | 10,335,950 | 11,215,290 | 10,659,650 |
| HMDB01058 | bisphosphate | 12,861,620 | 14,908,030 | 15,030,150 | 10,176,490 | 12,986,490 | 13,433,710 |
| HMDB00254 | Succinic acid | 10,283,610 | 9,620,985 | 8,567,130 | 9,808,415 | 11,432,620 | 14,473,460 |
| HMDB00191 | L-Aspartic acid | 15,081,320 | 14,683,660 | 13,148,890 | 9,529,167 | 11,274,240 | 16,105,810 |
| HMDB00239 | Pyridoxine | 8,832,328 | 8,611,456 | 8,382,888 | 9,272,513 | 10,102,820 | 10,351,980 |
| HMDB00765 | Mannitol | 10,121,930 | 8,419,663 | 8,681,563 | 9,124,205 | 9,515,589 | 10,944,240 |
| HMDB00134 | Fumaric acid | 13,445,950 | 13,952,500 | 13,363,700 | 8,464,455 | 9,727,776 | 11,821,440 |
| HMDB01138 | N-Acetylglutamic acid | 8,871,362 | 7,697,575 | 8,104,613 | 7,524,119 | 8,837,337 | 8,010,711 |
| HMDB00538 | Adenosine triphosphate Adenosine | 4,943,762 | 5,495,154 | 5,245,521 | 7,254,237 | 6,217,538 | 7,426,501 |
| HMDB00045 | monophosphate | 8,070,902 | 8,069,026 | 9,035,150 | 6,997,849 | 8,959,282 | 6,609,897 |
| HMDB01185 | S-Adenosylmethionine | 11,557,140 | 9,561,114 | 9,073,711 | 6,948,937 | 8,437,885 | 9,944,495 |
| HMDB00965 | Hypotaurine D-Glyceraldehyde 3- | 6,640,697 | 6,626,889 | 6,612,347 | 6,754,939 | 7,052,286 | 8,446,435 |
| HMDB01112 | phosphate | 6,400,597 | 4,119,763 | 3,266,154 | 6,590,523 | 2,998,173 | 3,562,678 |
| HMDB00660 | D-Fructose Asymmetric | 4,924,201 | 4,814,986 | 4,739,201 | 6,135,849 | 6,547,945 | 7,210,193 |
| HMDB01539 | dimethylarginine | 6,360,740 | 5,717,609 | 6,064,635 | 5,492,503 | 6,220,486 | 6,753,139 |
| HMDB00510 | Aminoadipic acid | 4,780,156 | 5,137,502 | 5,032,641 | 5,352,894 | 5,983,553 | 6,501,110 |
| HMDB00005 | 2-Ketobutyric acid | 7,502,768 | 7,593,036 | 7,357,433 | 5,289,178 | 6,027,442 | 5,724,353 |
| HMDB02064 | N-Acetylputrescine | 14,850,660 | 15,303,690 | 13,779,120 | 5,287,508 | 7,257,189 | 5,943,667 |
| HMDB01341 | ADP Uridine diphosphate | 4,193,646 | 4,729,147 | 5,320,192 | 4,962,730 | 5,529,189 | 5,002,783 |
| HMDB00286 | glucose | 7,094,001 | 7,271,041 | 7,562,056 | 4,959,327 | 5,703,347 | 6,869,287 |
| HMDB03337 | Oxidized glutathione | 3,209,322 | 3,177,918 | 3,115,170 | 3,793,739 | 4,422,168 | 4,949,576 |
| HMDB00766 | N-Acetyl-L-alanine | 2,979,459 | 2,980,116 | 3,089,901 | 3,499,773 | 3,556,484 | 4,596,263 |
| HMDB00568 | D-Arabitol D-2-Hydroxyglutaric | 4,211,463 | 4,181,490 | 3,846,771 | 3,445,547 | 3,545,938 | 4,135,015 |
| HMDB00606 | acid | 6,005,432 | 5,721,952 | 5,259,765 | 3,167,321 | 3,985,443 | 4,584,429 |
| HMDB01890 | Acetylcysteine | 2,962,755 | 2,838,837 | 3,757,806 | 3,133,666 | 3,527,413 | 3,894,968 |
| HMDB00295 | Uridine 5′-diphosphate | 2,159,430 | 2,862,990 | 3,531,439 | 2,964,858 | 3,462,504 | 2,781,952 |
| HMDB00124 | Fructose 6-phosphate | 2,454,536 | 2,476,637 | 2,365,664 | 2,660,942 | 2,572,195 | 2,965,940 |
| HMDB00289 | Uric acid | 1,435,632 | 1,017,624 | 1,528,017 | 2,415,527 | 1,430,740 | 933,580 |
| HMDB00243 | Pyruvic acid | 3,545,789 | 3,559,020 | 3,181,641 | 2,402,001 | 2,700,020 | 2,237,894 |
| HMDB11745 | N-Acetyl-L-methionine | 1,361,546 | 1,915,549 | 2,082,060 | 2,318,960 | 1,894,539 | 2,276,345 |
| HMDB00033 | Carnosine | 2,034,812 | 2,087,205 | 2,259,870 | 2,256,265 | 2,245,049 | 2,446,163 |
| HMDB01015 | N-Formyl-L-methionine | 1,058,614 | 1,625,306 | 1,643,452 | 2,242,009 | 2,343,563 | 2,318,203 |
| HMDB00283 | D-Ribose | 2,182,335 | 2,257,393 | 1,736,778 | 2,183,962 | 2,605,392 | 2,639,392 |
| HMDB00195 | Inosine D-2,3-Dihydroxypropanoic | 357,637 | 408,762 | 392,159 | 2,168,084 | 318,912 | 236,433 |
| HMDB31818 | acid | 2,058,890 | 2,092,979 | 2,059,011 | 2,140,143 | 2,437,835 | 2,431,980 |
| HMDB01066 | S-Lactoylglutathione | 1,520,102 | 3,020,546 | 2,957,792 | 2,114,224 | 2,485,483 | 1,817,914 |
| HMDB01401 | Glucose 6-phosphate | 3,226,415 | 3,548,032 | 3,418,089 | 2,103,241 | 2,833,469 | 3,624,968 |
| HMDB00807 | 3-Phosphoglyceric acid | 578,439 | 903,125 | 932,384 | 2,076,132 | 820,562 | 843,315 |
| HMDB01406 | Niacinamide | 1,677,335 | 1,384,551 | 1,503,894 | 1,982,720 | 1,686,510 | 1,871,343 |
| HMDB00292 | Xanthine | 1,763,468 | 1,232,465 | 1,416,890 | 1,967,615 | 1,894,384 | 2,180,514 |
| HMDB06029 | N-Acetylglutamine | 1,901,582 | 2,012,132 | 2,149,224 | 1,923,159 | 2,058,882 | 2,006,294 |
| HMDB00296 | Uridine | 343,732 | 437,217 | 427,430 | 1,893,519 | 318,302 | 299,157 |
| HMDB00821 | Phenylacetylglycine | 1,346,642 | 1,275,354 | 1,343,612 | 1,773,254 | 1,725,235 | 1,760,408 |
| HMDB01173 | 5′-Methylthioadenosine | 2,750,415 | 2,369,636 | 2,750,865 | 1,678,515 | 1,980,683 | 3,013,280 |
| HMDB11718 | 4-Hydroxybenzaldehyde N6,N6,N6-Trimethyl-L- | 1,472,908 | 1,382,783 | 1,308,004 | 1,623,875 | 1,610,244 | 2,163,467 |
| HMDB01325 | lysine | 1,574,082 | 1,380,084 | 1,271,807 | 1,450,139 | 1,666,314 | 1,913,626 |
| HMDB00462 | Allantoin Methylimidazoleacetic | 1,243,867 | 1,165,011 | 1,331,357 | 1,362,548 | 1,445,842 | 1,605,951 |
| HMDB02820 | acid | 833,806 | 1,026,019 | 1,081,972 | 1,358,938 | 1,418,950 | 1,508,563 |
| HMDB02005 | Methionine sulfoxide | 1,328,291 | 1,117,868 | 1,162,510 | 1,343,863 | 1,483,506 | 1,747,311 |
| HMDB00285 | Uridine triphosphate N-Acetyl-glucosamine | 1,113,088 | 1,277,985 | 1,415,333 | 1,308,516 | 1,268,414 | 1,538,242 |
| HMDB01367 | 1-phosphate | 784,289 | 815,286 | 811,274 | 1,300,461 | 800,586 | 850,336 |
| HMDB00034 | Adenine | 1,207,159 | 1,384,759 | 1,011,892 | 1,219,554 | 812,836 | 907,390 |
| HMDB00828 | Ureidosuccinic acid Phosphoenolpyruvic | 2,013,658 | 2,026,905 | 1,845,249 | 1,132,717 | 1,612,814 | 1,811,192 |
| HMDB00263 | acid | 513,954 | 547,828 | 550,522 | 1,119,695 | 505,583 | 468,849 |
| HMDB01227 | 5-Thymidylic acid | 1,198,012 | 1,137,602 | 1,264,131 | 1,061,384 | 1,368,086 | 1,260,084 |
| HMDB00262 | Thymine | 843,640 | 825,900 | 695,445 | 962,069 | 884,966 | 960,234 |
| HMDB00214 | Ornithine Uridine 5′- | 746,709 | 746,027 | 702,758 | 960,744 | 1,035,848 | 1,261,077 |
| HMDB00288 | monophosphate | 1,170,005 | 1,315,310 | 1,469,916 | 904,984 | 1,359,673 | 975,233 |
| HMDB00126 | Glycerol3-phosphate Cytidine | 841,790 | 820,747 | 763,213 | 758,556 | 825,062 | 1,025,018 |
| HMDB00095 | monophosphate | 995,129 | 997,487 | 970,562 | 724,977 | 871,719 | 625,602 |
| HMDB00186 | Alpha-Lactose | 724,857 | 624,193 | 655,668 | 636,775 | 666,969 | 547,770 |
| HMDB01548 | D-Ribose 5-phosphate | 1,005,922 | 882,956 | 792,338 | 624,479 | 581,839 | 786,584 |
| HMDB01863 | 2-Hydroxyvaleric acid | 833,128 | 843,183 | 754,829 | 621,532 | 904,968 | 786,820 |
| HMDB00133 | Guanosine | 169,216 | 207,673 | 200,723 | 619,336 | 155,195 | 131,494 |
| HMDB02994 | Erythritol | 515,833 | 511,699 | 498,673 | 598,716 | 590,438 | 628,349 |
| HMDB01487 | NADH D-Sedoheptulose 7- | 101,342 | 114,019 | 107,013 | 583,067 | 148,307 | 116,715 |
| HMDB01068 | phosphate | 679,599 | 618,793 | 613,796 | 561,321 | 572,202 | 625,243 |
| HMDB01546 | CDP | 474,406 | 592,589 | 663,673 | 536,936 | 604,322 | 492,208 |
| HMDB01254 | Glucosamine 6-phosphate | 1,112,510 | 1,032,812 | 1,051,627 | 529,990 | 692,898 | 945,530 |
| HMDB00939 | 5-Adenosylhomocysteine | 295,746 | 516,896 | 894,944 | 502,295 | 543,231 | 555,202 |
| HMDB01201 | Guanosine diphosphate | 482,721 | 579,876 | 646,899 | 493,573 | 471,968 | 538,827 |
| HMDB01409 | dUMP | 566,503 | 536,115 | 568,748 | 484,834 | 490,872 | 603,149 |
| HMDB01067 | N-Acetylaspartylglutamic acid | 670,056 | 698,083 | 728,110 | 473,464 | 613,919 | 690,837 |
| HMDB00482 | Caprylic acid Guanosine | 711,161 | 541,660 | 492,053 | 470,899 | 477,590 | 472,338 |
| HMDB01397 | monophosphate 4-Acetamidobutanoic | 801,004 | 795,263 | 804,601 | 465,594 | 655,797 | 560,973 |
| HMDB03681 | acid | 403,101 | 387,683 | 403,551 | 438,862 | 501,312 | 487,324 |
| HMDB00052 | Argininosuccinic acid | 652,280 | 637,067 | 668,335 | 397,796 | 561,328 | 632,101 |
| HMDB01316 | 6-Phosphogluconic acid | 559,964 | 608,301 | 620,404 | 394,956 | 515,553 | 654,253 |
| HMDB00099 | L-Cystathionine | 428,131 | 440,817 | 434,043 | 371,785 | 449,627 | 589,036 |
| HMDB01413 | Citicoline | 775,689 | 764,464 | 19,408 | 361,538 | 444,892 | 574,461 |
| HMDB00301 | Urocanic acid | 349,259 | 412,634 | 260,313 | 334,329 | 651,672 | 192,858 |
| HMDB00226 | Orotic acid | 623,346 | 515,743 | 558,819 | 303,308 | 293,998 | 226,604 |
| HMDB03349 | L-Dihydroorotic acid | 686,169 | 701,263 | 664,950 | 281,053 | 429,139 | 448,915 |
| HMDB00684 | L-Kynurenine | 268,447 | 271,566 | 252,063 | 280,930 | 306,374 | 392,315 |
| HMDB03357 | N-Acetylornithine | 262,871 | 210,167 | 204,871 | 261,750 | 259,387 | 327,139 |
| HMDB01624 | 2-Hydroxycaproic acid | 311,016 | 1,734,727 | 369,679 | 244,811 | 833,959 | 281,142 |
| HMDB00089 | Cytidine | 241,725 | 260,087 | 315,953 | 225,855 | 258,465 | 227,521 |
| HMDB01043 | Arachidonic acid | 87,289 | 88,185 | 74,271 | 219,569 | 129,238 | 117,457 |
| HMDB00157 | Hypoxanthine | 142,993 | 147,609 | 163,208 | 211,957 | 135,595 | 141,122 |
| HMDB01721 | DL-O-Phosphoserine | 61,108 | 73,785 | 66,764 | 185,294 | 163,584 | 141,059 |
| HMDB00082 | Cytidine triphosphate | 92,661 | 135,550 | 169,340 | 183,937 | 215,682 | 295,169 |
| HMDB00175 | Inosinic acid Thymidine 5′- | 483,642 | 475,691 | 418,678 | 178,233 | 246,521 | 243,587 |
| HMDB01342 | triphosphate | 79,487 | 83,614 | 77,797 | 177,094 | 147,940 | 156,549 |
| HMDB00273 | Thymidine | 47,339 | 42,481 | 39,415 | 143,606 | 96,280 | 76,717 |
| HMDB02320 | Imidazolelactic acid | 224,902 | 212,461 | 185,172 | 128,804 | 165,439 | 221,241 |
| HMDB00206 | N6-Acetyl-L-lysine | 138,793 | 130,426 | 114,400 | 127,774 | 112,788 | 139,608 |
| HMDB00217 | NADP 3-hydroxyhexadecanoic | 90,343 | 112,481 | 115,069 | 120,286 | 98,434 | 96,478 |
| HMDB61658 | acid Sedoheptulose 1,7- | 193,917 | 197,617 | 348,216 | 119,550 | 182,677 | 362,311 |
| HMDB60274 | bisphosphate | 135,259 | 152,184 | 138,987 | 119,260 | 114,671 | 139,219 |
| HMDB00229 | Nicotinamide ribotide | 95,563 | 78,099 | 55,333 | 110,026 | 75,372 | 93,843 |
| HMDB01423 | Coenzyme A | 129,874 | 139,103 | 124,926 | 109,174 | 125,366 | 106,848 |
| HMDB01564 | CDP-Ethanolamine | 164,112 | 181,405 | 153,966 | 100,228 | 130,601 | 162,582 |
| HMDB03351 | GDP-glucose | 116,006 | 108,528 | 118,365 | 92,740 | 103,248 | 99,915 |
| HMDB06028 | N-Acetylasparagine (R)-3-Hydroxydecanoic | 85,234 | 71,877 | 4,675 | 90,904 | 92,805 | 102,592 |
| HMDB10725 | acid | 105,501 | 128,272 | 114,660 | 88,715 | 193,751 | 99,729 |
| HMDB06557 | ADP-glucose | 83,386 | 112,072 | 100,757 | 84,781 | 90,192 | 99,689 |
| HMDB00671 | Indolelactic acid | 58,835 | 45,846 | 57,228 | 84,732 | 90,304 | 155,173 |
| HMDB00244 | Riboflavin | 78,901 | 96,089 | 89,411 | 84,676 | 97,270 | 112,665 |
| HMDB00222 | L-Palmitoylcarnitine | 121,589 | 95,087 | 115,379 | 73,260 | 167,693 | 194,186 |
| HMDB00221 | NADPH | 85,238 | 71,262 | 74,849 | 71,492 | 67,909 | 76,928 |
| HMDB06116 | 3-Hydroxyhippuric acid | 49,641 | 65,964 | 47,032 | 67,709 | 54,023 | 55,195 |
| HMDB01209 | Allantoic acid | 39,509 | 40,990 | 36,125 | 66,783 | 51,760 | 61,927 |
| HMDB00779 | Phenyllactic acid | 78,801 | 61,399 | 68,255 | 59,693 | 75,824 | 89,853 |
| HMDB00715 | Kynurenic acid Deoxyribose 5- | 52,901 | 37,401 | 40,638 | 51,692 | 41,122 | 61,792 |
| HMDB01031 | phosphate | 60,723 | 78,094 | 52,006 | 50,751 | 42,917 | 52,112 |
| HMDB00714 | Hippuric acid | 44,540 | 35,830 | 36,100 | 47,961 | 38,230 | 36,149 |
| HMDB00951 | Taurochenodesoxycholic acid | 51,541 | 32,351 | 30,162 | 47,031 | 60,620 | 60,122 |
| HMDB00012 | Deoxyuridine | 25,659 | 19,885 | 27,191 | 40,015 | 22,277 | 25,003 |
| HMDB01554 | Xanthylic acid | 10,347 | 9,995 | 17,342 | 36,455 | 11,062 | 9,617 |
| HMDB01107 | 7-Methylguanosine | 18,805 | 23,963 | 29,372 | 25,667 | 34,657 | 32,703 |
| HMDB00071 | Deoxyinosine | - | 14,683 | 6,806 | 8,871 | 6,834 | 15,777 |
| HMDB01022 | Succinyl-CoA Beta-D- | - | - | - | - | - | - |
| HMDB10314 | Glucopyranuronic acid | - | - | - | - | - | - |
| HMDB00192 | L-Cystine | - | - | - | - | - | - |
| HMDB00030 | Biotin | 35,471 | 21,537 | 22,439 | - | 28,122 | 15,310 |
| HMDB00764 | Hydrocinnamic acid | - | - | - | - | - | - |
| HMDB00699 | 1-Methylnicotinamide | - | - | - | - | - | - |
| HMDB00150 | Gluconolactone | - | - | - | - | - | - |
| HMDB00017 | 4-Pyridoxic acid | - | - | - | - | - | - |
| HMDB01245 | dCDP | - | - | - | - | - | - |
| HMDB00705 | Hexanoylcarnitine Thiamine | - | - | - | - | - | - |
| HMDB02666 | monophosphate | - | - | - | - | - | - |
| HMDB00044 | Ascorbic acid6-Phosphonoglucono- | - | - | - | - | - | - |
| HMDB01127 | D-lactone | - | - | - | - | - | - |
| HMDB00300 | Uracil | - | - | - | - | - | - |
| HMDB00653 | Cholesterol sulfate | - | - | - | - | - | - |
| HMDB00223 | Oxalacetic acid | - | - | - | - | - | - |
| HMDB02302 | 3-Indolepropionic acid | - | - | - | - | - | - |
| HMDB01202 | dCMP | - | - | - | - | - | - |
| HMDB00619 | Cholic acid | - | - | - | - | - | - |
| HMDB13713 | N-acetyltryptophan | - | - | - | - | - | - |
| HMDB00101 | Deoxyadenosine | - | - | - | - | - | - |
| HMDB01517 | AICAR | 126,084 | 153,152 | 165,534 | - | 32,080 | 59,426 |
| HMDB00132 | Guanine | - | - | - | - | - | - |
| HMDB01273 | Guanosine triphosphate | - | - | - | - | - | - |
| HMDB01124 | Trehalose 6-phosphate | - | - | - | - | - | - |
| HMDB00630 | Cytosine | - | - | - | - | - | - |
| HMDB00310 | Methylacetoacetic acid | - | - | - | - | - | - |
| HMDB01248 | FAD Indole-3-carboxylic | - | - | - | - | - | - |
| HMDB03320 | acid | - | - | - | - | - | - |
| HMDB00710 | 4-Hydroxybutyric acid Deoxyadenosine | - | - | - | - | - | - |
| HMDB00905 | monophosphateDeoxyuridine | - | - | - | - | - | - |
| HMDB01191 | triphosphate | - | - | - | - | - | - |
| HMDB00742 | Homocysteine3 -Methylthiopropionic | - | - | - | - | - | - |
| HMDB01527 | acid | - | - | - | - | - | - |
| HMDB00128 | Guanidoacetic acidDeoxyadenosine | - | - | - | - | - | - |
| HMDB01532 | triphosphate | - | - | - | - | - | - |
| HMDB01206 | Acetyl-CoA | - | - | - | - | - | - |
| HMDB01520 | Flavin Mononucleotide | - | - | - | - | - | - |
| HMDB00881 | Xanthurenic acidD-Erythrose 4- | - | - | - | - | - | - |
| HMDB01321 | phosphate | - | - | - | - | - | - |
| HMDB00998 | dCTP | 6,850 | 7,388 | 6,437 | - | - | 7,042 |
| HMDB00161 | L-Alanine 2-Oxo-4- | - | - | - | - | - | - |
| HMDB01553 | methylthiobutanoic acid | - | - | - | - | -- | - |
| HMDB00574 | L-Cysteine | - | - | - | - | - | - |
| HMDB00682 | Indoxyl sulfate | - | - | - | - | - | - |
| HMDB03335 | IDP | - | - | - | - | - | - |
| HMDB00038 | Dihydrobiopterin | - | - | - | - | - | - |
| HMDB03331 | 1-Methyladenosine | - | - | - | - | - | - |
| HMDB01514 | GlucosaminePhosphoribosyl | - | - | - | - | - | - |
| HMDB00280 | pyrophosphate | - | - | - | - | - | - |
| HMDB01586 | Glucose 1-phosphate | - | - | - | - | - | - |
| HMDB00738 | Indole | - | - | - | - | - | - |
| HMDB06372 | L-Glyceric acidUridine diphosphate | - | - | - | - | - | - |
| HMDB00935 | glucuronic acid | - | - | - | - | - | - |
| HMDB01256 | Spermine | - | - | - | - | - | - |
| HMDB01366 | PurineAdenosine | - | - | - | - | - | - |
| HMDB01003 | phosphosulfate | - | - | - | - | - | - |
| HMDB02339 | 5-Methoxytryptophan N-Acetyl-D- | - | - | - | - | - | - |
| HMDB00215 | glucosamine | - | - | - | - | - | - |
| HMDB00058 | Cyclic AMP | - | - | - | - | - | - |

TABLE 5

Pathway topology analysis using the 9 common metabolites significantly downregulated by CO in both MCF7/TGL and MDA-MB-231/TGL cell lines

| Pathway Name | Match Status | p | -log(p) | Holm p | FDR | Impact | Details |
|---|---|---|---|---|---|---|---|
| Citrate cycle (TCA cycle) | 3/20 | 1.471E-4 | 8.8244 | 0.012357 | 0.012357 | 0.13471 | KEGG SMP |
| Alanine, aspartate and glutamate metabolism | 3/28 | 4.1292E-4 | 7.7922 | 0.034273 | 0.017343 | 0.05048 | KEGG SMP SMP SMP |
| Arginine biosynthesis | 2/14 | 0.0026318 | 5.9401 | 0.2158 | 0.073689 | 0.0 | KEGG |
| Pvruvate metabolism | 2/22 | 0.006521 | 5.0327 | 0.5282 | 0.13694 | 0.20684 | KEGG SMP |
| Tyrosine metabolism | 2/42 | 0.022877 | 3.7776 | 1.0 | 0.38434 | 0.02463 | KEGG SMP SMP |
| D-Glutamine and D- glutamate metabolism | ⅙ | 0.034392 | 3.3699 | 1.0 | 0.48148 | 0.0 | KEGG SMP |
| Butanoate metabolism | 1/15 | 0.084009 | 2.4768 | 1.0 | 0.88209 | 0.0 | KEGG SMP |
| Nicotinate and nicotinamide metabolism | 1/15 | 0.084009 | 2.4768 | 1.0 | 0.88209 | 0.23465 | KEGG SMP |
| Glycolysis/ Gluconeogenesis | 1/26 | 0.14157 | 1.955 | 1.0 | 1.0 | 0.10044 | KEGG SMP SMP |
| Glvoxylate and dicaiboxylate metabolism | 1/32 | 0.17159 | 1.7627 | 1.0 | 1.0 | 0.0 | KEGG |
| Glycine, serine and threonine metabolism | 1/33 | 0.1765 | 1.7344 | 1.0 | 1.0 | 0.0 | KEGG SMP |
| Cysteine and methionine metabolism | 1/33 | 0.1765 | 1.7344 | 1.0 | 1.0 | 0.0 | KEGG SMP SMP |
| Arginine and proline metabolism | 1/38 | 0.20067 | 1.6061 | 1.0 | 1.0 | 0.0 | KEGG SMP |
| Pyrimidine metabolism | 1/39 | 0.20543 | 1.5827 | 1.0 | 1.0 | 0.03727 | KEGG SMP |
| Purine metabolism | 1/65 | 0.32062 | 1.1375 | 1.0 | 1.0 | 0.13277 | KEGG SMP |

Accordingly, compositions including low doses of carbon monoxide as disclosed herein are useful in methods for treating or preventing metastasis in a subject in need thereof.

### Example 9: TCA Cycle and Cellular Heme Levels

The TCA cycle plays an important role in many biochemical pathways and provides precursors used in numerous other reactions. One of the downstream reactions is the biosynthesis of heme, (iron protoporphyrin IX), an essential iron-containing molecule. Free heme (Fe²⁺ state) is readily oxidized, so oxidized heme levels in the form of hemin (Fe³⁺ state) are used to assess intracellular heme levels.

Cellular heme/hemin levels were examined and it was found that 250 ppm CO reduced heme/hemin levels (FIG. 4C) and correlated with reduced migration compared to untreated controls (FIG. 1A and FIG. 1C). In addition, 250 ppm CO reduced the expression of heme transporters, HRG1 and HCP1 (FIG. 4D), suggesting CO also impaired heme uptake. To determine whether supplementing heme/hemin can reverse the CO inhibitory effect on tumor cell migration, 30 µM hemin was added to both the upper and lower chambers for transwell migration assays. 30 µM hemin significantly reversed cell migration inhibited by 250 ppm CO in MCF7/TGL, HCC1954, MDA-MB-231/TGL, 8988T, CM, SW480, 22Rvl, HepG2 and H1975 cells (FIGS. 1A-1I), but did not alter cancer cell proliferation (FIGS. 2A-2I).

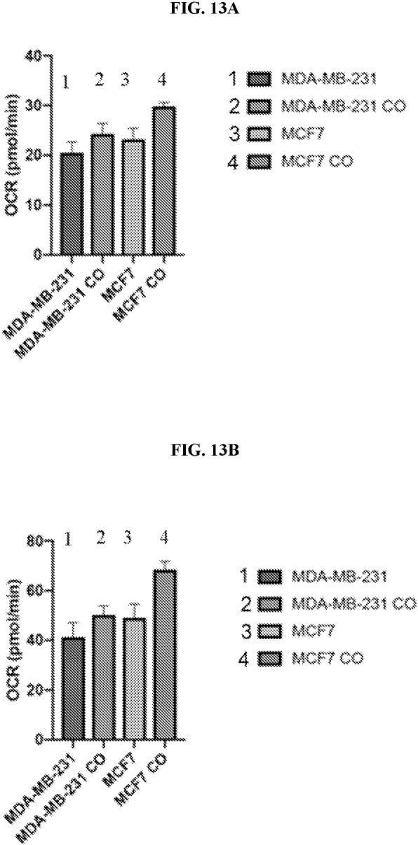

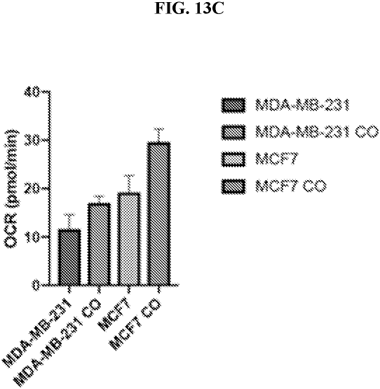

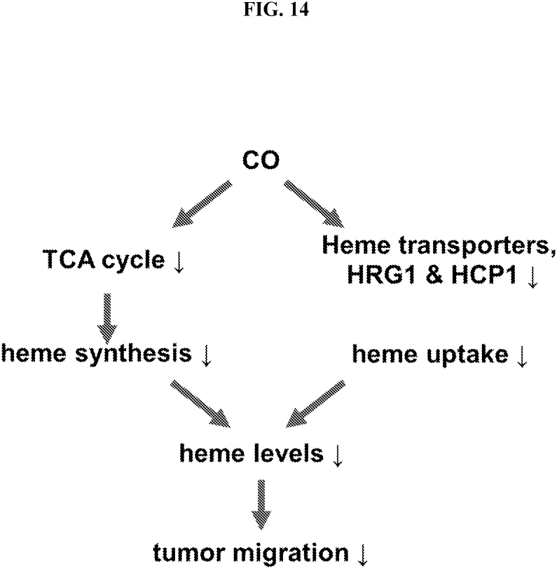

Low dose carbon monoxide treatment did not affect mitochondrial ATP production and stress-induced mitochondrial maximum respiration in MDA-MB-231 and MCF7 cells (FIGS. 13A-13B). Carbon monoxide treatment improved mitochondrial spare respiratory capacity in MDA-MB-231 and MCF7 cells (FIG. 13C).

Accordingly, compositions including low doses of carbon monoxide as disclosed herein are useful in methods for treating or preventing metastasis in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating or preventing metastasis in a subject in need thereof, comprising administering to the subject an effective amount of carbon monoxide at a low dose of about 100 ppm to about 300 ppm.

2. The method of claim 1, wherein the subject is diagnosed with or is suffering from breast cancer, lung cancer, colon cancer, rectal cancer, prostate cancer, pancreatic cancer, liver cancer, kidney and renal cancer, brain and other nervous system tumors, head and neck cancer, neuroendocrine tumor, blood cancer, gynecologic malignancies, or urinary bladder cancer, optionally wherein the breast cancer is an estrogen receptor negative (ER⁻) breast cancer, an estrogen receptor positive (ER⁺) breast cancer, a progesterone receptor negative breast cancer (PR⁻), a progesterone receptor positive (PR⁺) breast cancer, a Her2⁺ breast cancer, or a triple negative (ER⁻/PR⁻/Her2⁻) breast cancer.

3. (canceled)

4. The method of claim 1 wherein the subject exhibits at least one mutation in one or more genes selected from the group consisting of BARD1, BRCAI, BRCA2, PALB2, RAD51D, BRIP1, RAD51C, ESRI, BCL2, ABRAXASI, AIP, ALK, APC, ATM, AXIN2, BAP 1, BLM, BMPR1A, CASR, CDC73, CDH1, CDK4, CDKNIB, CDKN2A, CFTR, CHEK2, CPA1, CTNNAI, CTRC, DICERI, EGFR, EGLN1, EPCAM, FANCC, FH, FLCN, GALNT12, GREMI, HOXB13, KIFIB, KIT, LZTRI, MAX, MEN1, MET, MITF, MLH1, MLH3, MRE11, MSH2, MSH3, MSH6, MUTYH, NBN, NF1, NF2, NTHL1, PALLD, PDGFRA, PHOX2B, PMS2, POLD1, POLE, POT1, PRKARIA, PRSS1, PTCH1, PTEN, RAD50, RB1, RECQL, RET, RINT1, RPS20, SDHA, SDHAF2, SDHB, SDHC, SDHD, SMAD4, SMARCA4, SMARCB1, SMARCE1, SPINK1, STK11, SUFU, TERT, TMEM127, TP53, TSC1, TSC2, VHL, and XRCC2; or
wherein the subject exhibits at least one symptom selected from the group consisting of persistent cough, bloody phlegm, chest pain, shortness of breath, wheezing, weakness, sudden weight loss, bone pain, bone fractures, urinary incontinence, bowel incontinence, hypercalcemia, nausea, vomiting, constipation, confusion, headache, seizures, dizziness, numbness in the face, arms or legs, memory loss, changes in behaviour and personality, loss of balance and coordination, problems with speech and/or swallowing, abdominal pain, pain occurring near the right shoulder blade or in the upper abdomen, loss of appetite, abdominal swelling, jaundice, fatigue, and fever; or
wherein the subject exhibits over-expression of HMMR or a Bcl-2 family gene, optionally wherein the Bcl-2 family gene is BCL2L1; or
wherein the subject is human.

5. The method of claim 1, wherein the metastasis has developed in one or more organs selected from the group consisting of lymph nodes, liver, brain, lungs, kidney, bones, lymphatics cavity, peritoneal cavity, and thoracic cavity.

6. (canceled)

7. The method of claim 1, wherein the effective amount of low dose carbon monoxide is about 150 ppm to about 500 ppm carbon monoxide.

8. (canceled)

9. The method of claim 1, wherein administration of the effective amount of carbon monoxide
blocks metastasis and/or migration in estrogen receptor positive (ER⁺) breast cancer: or
does not reduce cancer cell proliferation; or
blocks migration, metastases and/or proliferation in triple negative breast cancer cells or liver cancer cells.

10. (canceled)

11. (canceled)

12. (canceled)

13. The method of claim 1, wherein the carbon monoxide is administered as or with at least one of a certified medical grade carbon monoxide gas, a recombumin-Ru^{II}(CO)₂ complex, a nanoparticle, or a carbon-monoxide releasing molecule (CORM), optionally wherein the CORM comprises a transition metal based CORM, an organic CORM, or a combination thereof.

14. (canceled)

15. The method of claim 13,
wherein the transition-metal based CORM comprises a metal carbonyl complex of formula [M(CO)ₓL_{y}]±z[Q]±p wherein:
(i) M is a d transition metal, optionally Mo, Mn, Re, Fe, Ru, Co;
(ii) x >1;
(iii) L_{y} represents one or more ancillary mono-or polydentate ligands comprising C, N, O, P, S, Se, donor atoms or one or more of the halides, F, Cl, Br, I, which together with the CO ligands provide the complex with a 16, 17, or 18 electron valence shell configuration;
(iv) z is the overall charge of the complex;
(v) Q is a counter-ion; and
(vi) p is an integer value such that the p± charge cancels the z± value; or
wherein the organic CORM comprises an organoborane or an organic molecule configured to release CO to a biological medium or an entity-like buffer, a culture media, blood, a cell, a tissue, an organ, a tumor or a mammal.

16. (canceled)

17. The method of claim 13, wherein the CORM releases CO by at least one of:
(i) spontaneous release upon dissolution;
(ii) action of a specific chemical or enzymatic trigger in the cell, tissue, organ or tumor;
(iii) exogenous action of another organic or inorganic chemical entity; or
(iv) exogenous action of physical stimuli such as light, heat, electric or magnetic fields; or
wherein the CORM comprises dichloromethane, sodium boranocarbonate, tricarbonyldichlororuthenium (II) dimer, tricarbonylchloro(glycinato)ruthenium (II), [Me₄N][Mn(CO)₄(thioacetate)₂], dimanganese decacarbonyl, iron pentacarbonyl, or any combination thereof.

18. (canceled)

19. The method of claim 13, wherein the nanoparticles comprise liposomes, biodegradable polylactic acid (“PLA”), biodegradable polyglycolic acid (“PGA”), ultrasound contrast microbubbles, or biodegradable poly(lactic-co-glycolic acid) (“PGLA”).

20. The method of claim 1, wherein the carbon monoxide is administered to the subject separately, sequentially or simultaneously with an additional therapeutic agent, optionally wherein the additional therapeutic agent is selected from the group consisting of alkylating agents, topoisomerase inhibitors, endoplasmic reticulum stress inducing agents, antimetabolites, immunotherapeutic agents, mitotic inhibitors, nitrogen mustards, nitrosoureas, alkylsulfonates, platinum agents, taxanes, vinca agents, anti-estrogen drugs, aromatase inhibitors, VEGF/VEGFR inhibitors, EGF/EGFR inhibitors, PARP inhibitors, cytostatic alkaloids, cytotoxic antibiotics, endocrine/hormonal agents, bisphosphonate therapy agents, phenphormin, anti-angiogenic agents, Histone deacetylase inhibitors, and non-steroidal anti-inflammatory drugs (NSAIDs).

21. (canceled)

22. The method of claim 20, wherein the additional therapeutic agent is a chemotherapeutic agent selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, ABRAXANE^{®} (albumin-bound paclitaxel), protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, alpelisib, and mixtures thereof; or
wherein the additional therapeutic agent is an antimetabolite selected from the group consisting of 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof; or
wherein the additional therapeutic agent is a taxane selected from the group consisting of accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof; or
wherein the additional therapeutic agent is a DNA alkylating agent selected from the group consisting of cyclophosphamide, chlorambucil, melphalan, bendamustine, uramustine, estramustine, carmustine, lomustine, nimustine, ranimustine, streptozotocin, busulfan, mannosulfan, and mixtures thereof; or
wherein the additional therapeutic agent is a topoisomerase I inhibitor selected from the group consisting of SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof; or
wherein the additional therapeutic agent is a topoisomerase II inhibitor selected from the group consisting of amsacrine, etoposide, etoposide phosphate, teniposide, daunorubicin, mitoxantrone, ellipticines, aurintricarboxylic acid, doxorubicin, and HU-331 and combinations thereof; or
wherein the additional therapeutic agent is an immunotherapeutic agent selected from the group consisting of immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX; or
wherein the additional therapeutic agent is an anti-angiogenic agent selected from the group consisting of bevacizumab, cediranib, axitinib, anginex, sunitinib, sorafenib, pazopanib, vatalanib, cabozantinib, ponatinib, lenvatinib, SU6668, Everolimus (Afinitor^{®}), Lenalidomide (Revlimid^{®}), Ramucirumab (Cyramza^{®}), Regorafenib (Stivarga^{®}), Thalidomide (Synovir, Thalomid^{®}), Vandetanib (Caprelsa^{®}), and Ziv-aflibercept (Zaltrap^{®}); or
wherein the additional therapeutic agent is a Histone deacetylase inhibitor selected from the group consisting of trichostatin A (TSA), tubacin, apicidin, depsipeptide, MS275, BML-210, RGFP966, MGCD0103, LBH589, splitomicin, FK228, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC202, Mocetinostat, Romidepsin, Nicotinamide, Sirtinol, Cambinol, and EX-527.

23. (canceled)

24. (canceled)

25. (canceled)

26. (canceled)

27. (canceled)

28. (canceled)

29. (canceled)

30. (canceled)

31. The method of claim 2, wherein administration of the effective amount of carbon monoxide results in decreased levels of one or more tricarboxylic acid (TCA) cycle metabolites in cancer cells compared to untreated cancer cells.

32. The method of claim 31, wherein the one or more TCA cycle metabolites are selected from the group consisting of fumaric acid, L-Dihydroorotic acid, D-2-Hydroxyglutaric, malic acid, NAD, GDP-glucose, pyruvic acid, inosinic acid, cis-aconitate, succinic acid, succinyl-coA, and oxoglutaric acid.

33. The method of claim 2, wherein administration of the effective amount of carbon monoxide results in reduced heme uptake or reduced heme biosynthesis in cancer cells compared to untreated cancer cells.

34. The method of claim 2 wherein administration of the effective amount of carbon monoxide results in decreased expression levels of HRG1, CYGB (Cytoglobin), CYP1B1 (Cytochrome P450 Family 1 Subfamily B Member 1), HCP1, SP1, WNT/beta-catenin, MYC, MYC target genes, and/or E2F target genes in cancer cells compared to untreated cancer cells.
